SEQUENCE LISTING (59 sequences)

SEQ ID NO: 1 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 2 - protein, 508 aa, Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 3 - protein, 728 aa, Psilocybe cubensis
MASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 4 - protein, 362 aa, Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 5 - protein, 309 aa, Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 6 - DNA, 1503 bp, Catharanthus roseus
atgggttctattgattctaccaacgtcgctatgtctaattctccagttggtgaatttaagccattggaagccgaagaatttagaaagcaagctcacagaatggttgatttcattgccgattactacaagaacgttgaaacctacccagttttgtctgaagttgaaccaggttacttgagaaagagaataccagaaactgctccatatttgccagaaccattggatgatattatgaaggacatccaaaaggacatcattccaggtatgactaattggatgtctccaaacttttacgctttcttccctgctactgtttcttctgctgcttttttgggtgaaatgttgtctactgctttgaactctgttggtttcacttgggtttcttcaccagctgctactgaattggaaatgatagttatggattggttggcccaaatcttgaagttgccaaagtcttttatgttctctggtactggtggtggtgttattcaaaacactacctccgaatctattttgtgcaccattattgctgctagagaaagagctttggaaaagttgggtccagattccattggtaaattggtttgttacggttctgatcaaacccataccatgtttccaaagacttgtaaattggctggtatctacccaaacaacatcagattgattccaaccactgttgaaaccgatttcggtatttctccacaagttttaagaaagatggtcgaagatgatgttgctgctggttatgttcctttgtttttgtgtgctactttgggtactacttctactactgctacagatccagttgactccttgtctgaaattgctaacgaatttggtatctggattcatgttgatgctgcttacgctggttctgcttgtatttgtcctgaatttagacactacttggacggtatcgaaagagtcgattctttgtctttgtctccacataagtggttgttggcttatttggattgcacttgtttgtgggttaagcaaccacatttgttgttgagagctttgactaccaatccagaatacttgaagaacaagcaatccgatttggataaggtcgttgactttaagaactggcaaattgctactggtagaaagttcagatccttgaaattgtggttgatcttgagatcctacggtgttgttaacttgcaatcccacattagatcagatgttgctatgggtaagatgtttgaagaatgggttagatccgactccagattcgaaatagttgttccaagaaacttctcattggtctgctttagattgaagccagatgtttcctcattgcacgttgaagaagtcaacaaaaagttgttggacatgttgaactctacaggtagagtctacatgactcatacaatagtcggtggtatctatatgttgagattggctgttggttcttcattgaccgaagaacatcatgttagaagagtttgggacttgatccaaaagttgaccgatgacttgttgaaagaagcctga

SEQ ID NO: 7 - DNA, 1527 bp, Psilocybe cubensis
atgatcgctgttttgttctctttcgttatcgctggttgtatctactacatcgtttctagaagagttagaagatctagattgccaccaggtccaccaggtatcccaatcccattcatcggtaacatgttcgacatgccagaagaatctccatggttgactttcttgcaatggggtagagactacaacactgacatcttgtacgttgacgctggtggtactgaaatggttatcttgaacactttggaaactatcactgacttgttggaaaagagaggttctatctactctggtagattggaatctactatggttaacgaattgatgggttgggaattcgacttgggtttcatcacttacggtgacagatggagagaagaaagaagaatgttcgctaaggaattctctgaaaagggtatcaagcaattcagacacgctcaagttaaggctgctcaccaattggttcaacaattgactaagactccagacagatgggctcaacacatcagacaccaaatcgctgctatgtctttggacatcggttacggtatcgacttggctgaagacgacccatggttggaagctactcacttggctaacgaaggtttggctatcgcttctgttccaggtaagttctgggttgactctttcccatctttgaagtacttgccagcttggttcccaggtgctgttttcaagagaaaggctaaggtttggagagaagctgctgaccacatggttgacatgccatacgaaactatgagaaagttggctccacaaggtttgactagaccatcttacgcttctgctagattgcaagctatggacttgaacggtgacttggaacaccaagaacacgttatcaagaacactgctgctgaagttaacgttggtggtggtgacactactgtttctgctatgtctgctttcatcttggctatggttaagtacccagaagttcaaagaaaggttcaagctgaattggacgctttgactaacaacggtcaaatcccagactacgacgaagaagacgactctttgccatacttgactgcttgtatcaaggaattgttcagatggaaccaaatcgctccattggctatcccacacaagttgatgaaggacgacgtttacagaggttacttgatcccaaagaacactttggttttcgctaacacttgggctgttttgaacgacccagaagtttacccagacccatctgttttcagaccagaaagatacttgggtccagacggtaagccagacaacactgttagagacccaagaaaggctgctttcggttacggtagaagaaactgtccaggtatccacttggctcaatctactgtttggatcgctggtgctactttgttgtctgctttcaacatcgaaagaccagttgaccaaaacggtaagccaatcgacatcccagctgacttcactactggtttcttcagacacccagttccattccaatgtagattcgttccaagaactgaacaagtttctcaatctgtttctggtccataa

SEQ ID NO: 8 - DNA, 2187 bp, Psilocybe cubensis
atggcttcttcttcttctgacgttttcgttttgggtttgggtgttgttttggctgctttgtacatcttcagagaccaattgttcgctgcttctaagccaaaggttgctccagtttctactactaagccagctaacggttctgctaacccaagagacttcatcgctaagatgaagcaaggtaagaagagaatcgttatcttctacggttctcaaactggtactgctgaagaatacgctatcagattggctaaggaagctaagcaaaagttcggtttggcttctttggtttgtgacccagaagaatacgacttcgaaaagttggaccaattgccagaagactctatcgctttcttcgttgttgctacttacggtgaaggtgaaccaactgacaacgctgttcaattgttgcaaaacttgcaagacgaatctttcgaattctcttctggtgaaagaaagttgtctggtttgaagtacgttgttttcggtttgggtaacaagacttacgaacactacaacttgatcggtagaactgttgacgctcaattggctaagatgggtgctatcagaatcggtgaaagaggtgaaggtgacgacgacaagtctatggaagaagactacttggaatggaaggacggtatgtgggaagctttcgctactgctatgggtgttgaagaaggtcaaggtggtgactctgctgacttcgttgtttctgaattggaatctcacccaccagaaaaggtttaccaaggtgaattctctgctagagctttgactaagactaagggtatccacgacgctaagaacccattcgctgctccaatcgctgttgctagagaattgttccaatctgttgttgacagaaactgtgttcacgttgaattcaacatcgaaggttctggtatcacttaccaacacggtgaccacgttggtttgtggccattgaacccagacgttgaagttgaaagattgttgtgtgttttgggtttggctgaaaagagagacgctgttatctctatcgaatctttggacccagctttggctaaggttccattcccagttccaactacttacggtgctgttttgagacactacatcgacatctctgctgttgctggtagacaaatcttgggtactttgtctaagttcgctccaactccagaagctgaagctttcttgagaaacttgaacactaacaaggaagaataccacaacgttgttgctaacggttgtttgaagttgggtgaaatcttgcaaatcgctactggtaacgacatcactgttccaccaactactgctaacactactaagtggccaatcccattcgacatcatcgtttctgctatcccaagattgcaaccaagatactactctatctcttcttctccaaagatccacccaaacactatccacgctactgttgttgttttgaagtacgaaaacgttccaactgaaccaatcccaagaaagtgggtttacggtgttggttctaacttcttgttgaacttgaagtacgctgttaacaaggaaccagttccatacatcactcaaaacggtgaacaaagagttggtgttccagaatacttgatcgctggtccaagaggttcttacaagactgaatctttctacaaggctccaatccacgttagaagatctactttcagattgccaactaacccaaagtctccagttatcatgatcggtccaggtactggtgttgctccattcagaggtttcgttcaagaaagagttgctttggctagaagatctatcgaaaagaacggtccagactctttggctgactggggtagaatctctttgttctacggttgtagaagatctgacgaagacttcttgtacaaggacgaatggccacaatacgaagctgaattgaagggtaagttcaagttgcactgtgctttctctagacaaaactacaagccagacggttctaagatctacgttcaagacttgatctgggaagacagagaacacatcgctgacgctatcttgaacggtaagggttacgtttacatctgtggtgaagctaagtctatgtctaagcaagttgaagaagttttggctaagatcttgggtgaagctaagggtggttctggtccagttgaaggtgttgctgaagttaagttgttgaaggaaagatctagattgatgttggacgtttggtcttaa

SEQ ID NO: 9 - DNA, 1089 bp, Psilocybe cubensis
atggctttcgacttgaagactgaagacggtttgatcacttacttgactaagcacttgtctttggacgttgacacttctggtgttaagagattgtctggtggtttcgttaacgttacttggagaatcaagttgaacgctccataccaaggtcacacttctatcatcttgaagcacgctcaaccacacatgtctactgacgaagacttcaagatcggtgttgaaagatctgtttacgaataccaagctatcaagttgatgatggctaacagagaagttttgggtggtgttgacggtatcgtttctgttccagaaggtttgaactacgacttggaaaacaacgctttgatcatgcaagacgttggtaagatgaagactttgttggactacgttactgctaagccaccattggctactgacatcgctagattggttggtactgaaatcggtggtttcgttgctagattgcacaacatcggtagagaaagaagagacgacccagaattcaagttcttctctggtaacatcgttggtagaactacttctgaccaattgtaccaaactatcatcccaaacgctgctaagtacggtgttgacgacccattgttgccaactgttgttaaggacttggttgacgacgttatgcactctgaagaaactttggttatggctgacttgtggtctggtaacatcttgttgcaattggaagaaggtaacccatctaagttgcaaaagatctacatcttggactgggaattgtgtaagtacggtccagcttctttggacttgggttacttcttgggtgactgttacttgatctctagattccaagacgaacaagttggtactactatgagacaagcttacttgcaatcttacgctagaacttctaagcactctatcaactacgctaaggttactgctggtatcgctgctcacatcgttatgtggactgacttcatgcaatggggttctgaagaagaaagaatcaacttcgttaagaagggtgttgctgctttccacgacgctagaggtaacaacgacaacggtgaaatcacttctactttgttgaaggaatcttctactgcttaa

SEQ ID NO: 10 - DNA, 930 bp, Psilocybe cubensis
atgcacatcagaaacccatacagaactccaatcgactaccaagctttgtctgaagctttcccaccattgaagccattcgtttctgttaacgctgacggtacttcttctgttgacttgactatcccagaagctcaaagagctttcactgctgctttgttgcacagagacttcggtttgactatgactatcccagaagacagattgtgtccaactgttccaaacagattgaactacgttttgtggatcgaagacatcttcaactacactaacaagactttgggtttgtctgacgacagaccaatcaagggtgttgacatcggtactggtgcttctgctatctacccaatgttggcttgtgctagattcaaggcttggtctatggttggtactgaagttgaaagaaagtgtatcgacactgctagattgaacgttgttgctaacaacttgcaagacagattgtctatcttggaaacttctatcgacggtccaatcttggttccaatcttcgaagctactgaagaatacgaatacgaattcactatgtgtaacccaccattctacgacggtgctgctgacatgcaaacttctgacgctgctaagggtttcggtttcggtgttggtgctccacactctggtactgttatcgaaatgtctactgaaggtggtgaatctgctttcgttgctcaaatggttagagaatctttgaagttgagaactagatgtagatggtacacttctaacttgggtaagttgaagtctttgaaggaaatcgttggtttgttgaaggaattggaaatctctaactacgctatcaacgaatacgttcaaggttctactagaagatacgctgttgcttggtctttcactgacatccaattgccagaagaattgtctagaccatctaacccagaattgtcttctttgttctaa

SEQ ID NO: 11 - protein, 207 aa, Bos taurus
MSTPSIHCLKPSPLHLPSGIPGSPGRQRRHTLPANEFRCLTPEDAAGVFEIEREAFISVSGNCPLNLDEVRHFLTLCPELSLGWFVEGRLVAFIIGSLWDEERLTQESLTLHRPGGRTAHLHALAVHHSFRQQGKGSVLLWRYLQHAGGQPAVRRAVLMCEDALVPFYQRFGFHPAGPCAVVVGSLTFTEMHCSLRGHAALRRNSDR

SEQ ID NO: 12 - DNA, 1113 bp, Saccharomyces cerevisiae
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 13 - DNA, 4767 bp, Saccharomyces cerevisiae
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 14 - DNA, 1131 bp, Saccharomyces cerevisiae
atgtcaacgtttgggaaactgttccgcgtcaccacatatggtgaatcgcattgtaagtctgtcggttgcattgtcgacggtgttcctccaggaatgtcattaaccgaagctgacattcagccacaattgaccagaagaagaccgggtcaatctaagctatcgacccctagagacgaaaaggatagagtggaaatccagtccggtaccgagttcggcaagactctaggtacacccatcgccatgatgatcaaaaacgaggaccaaagacctcacgactactccgacatggacaagttccctagaccttcccatgcggacttcacgtactcggaaaagtacggtatcaaggcctcctctggtggtggcagagcttctgctagagaaacgattggccgtgtcgcttcaggtgccattgctgagaagttcttagctcagaactctaatgtcgagatcgtagcctttgtgacacaaatcggggaaatcaagatgaacagagactctttcgatcctgaatttcagcatctgttgaacaccatcaccagggaaaaagtggactcaatgggtcctatcagatgtccagacgcctccgttgctggtttgatggtcaaggaaatcgaaaagtacagaggcaacaaggactctatcggtggtgtcgtcacttgtgtcgtgagaaacttgcctaccggtctcggtgagccatgctttgacaagttggaagccatgttggctcatgctatgttgtccattccagcatccaagggtttcgaaattggctcaggttttcagggtgtctctgttccagggtccaagcacaatgacccattttactttgaaaaagaaacaaacagattaagaacaaagaccaacaattcaggtggtgtacaaggtggtatctctaatggtgagaacatctatttctctgtcccattcaagtcagtggccactatctctcaagaacaaaaaaccgccacttacgatggtgaagaaggtatcttagccgctaagggtagacatgaccctgctgtcactccaagagctattcctattgtggaagccatgaccgctctggtgttggctgacgcgcttttgatccaaaaggcaagagatttctccagatccgtggttcattaa

SEQ ID NO: 15 - DNA, 675 bp, Saccharomyces cerevisiae
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 16 - DNA, 1524 bp, Saccharomyces cerevisiae
atgaccgcttccatcaaaattcaaccggatattgactctctaaagcaattacagcagcaaaatgacgatagttccataaatatgtatcccgtgtatgcgtatttgccatcattggatctgactcctcacgtggcatatctaaaattggcacaattgaacaaccctgatagaaaggaatcatttctgttggaaagtgctaagacaaataatgaattagatcgttattcattcataggtatctcgccacgcaagaccatcaaaaccggtcctactgaaggcattgaaacagatcctttggaaattttggaaaaggagatgtctacctttaaagtagccgaaaatgttcctggtttaccgaaattaagtggtggtgctattggttatatttcttatgactgtgttcgttatttcgagccaaaaacaagaaggcctttgaaagatgtcctaagacttccagaggcatatttaatgctttgtgataccattattgcctttgataatgtttttcagagatttcaaatcattcataacattaataccaatgaaacttcgttggaggaaggttaccaagctgcagcacaaataatcactgatatcgtatcaaagctaaccgacgattcctcgccaataccatatccagaacaacctcctattaaattgaatcaaacttttgaatcgaatgtgggcaaggaaggttacgaaaatcacgtctccactttgaagaagcatattaagaaaggtgatattattcaaggtgtgccatcgcaaagagtggcaaggccaacttcgttacatcctttcaatatttacagacatttacgtacagtgaacccatctccttacctgttttatattgattgtttggatttccaaatcattggtgcatctccagaattgttgtgcaaatcggattccaaaaatagagtcattacccatccaattgctggtactgtcaaacgtggggctactactgaagaggatgatgctttagcggaccaattacgtggctcgttaaaagaccgtgcagaacatgttatgctggtagatttagcaagaaacgatattaacagaatttgtgacccattaacaacaagtgtcgataaactgttaactattcaaaaattttctcatgtccaacatctggtttctcaagtcagcggtgttctccgcccagaaaagacaagatttgatgcattcagatcgattttccctgcaggtactgtcagtggtgctccaaaggttagagccatggaattgattgccgaactagaaggagaaaggcgtggggtttatgcaggcgccgtaggtcattggtcatacgacggtaaaacaatggacaattgtatcgctttaaggactatggtctataaagatggcattgcttacttgcaagctggcggtggtattgtttacgattcagatgagtacgatgaatatgtcgaaaccatgaataaaatgatggccaatcacagtactattgtgcaagcagaagaattgtgggccgatatcgtaggatcagcttaa

SEQ ID NO: 17 - DNA, 1455 bp, Saccharomyces cerevisiae
atgtctgtgcacgctgcaacaaacccaatcaataagcatgtggttctaattgacaactacgattcctttacctggaacgtttacgagtacttgtgccaggagggcgccaaagtgagcgtctaccgtaacgatgcaattacagttccagaaattgccgccttgaatcccgacacattgcttatctcgcctggaccaggccacccaaagacagattctggcatttcaagagactgtatccggtactttactgggaaaattcctgtatttggaatctgtatgggccagcaatgcatgtttgacgtatttggtggtgaagttgcctacgctggtgagattgtccacggtaaaacgtccccaatctctcacgacaactgtggaattttcaagaacgtgccgcaaggtattgctgtgacaagataccattcattggccgggacagaatcgtccctaccatcctgcttgaaggttactgcgagtaccgaaaatggaattatcatgggtgtaagacacaagaagtacactgtagaaggtgtgcaatttcatccggaatccatcttgaccgaggaaggtcatctgatgatcaggaacattttaaacgtcagtggaggcacttgggaggaaaacaaatcatctccttcaaattctattttggaccgtatctatgctcggcgtaaaatagacgtcaatgagcagtctaaaatcccaggtttcacctttcaagacttacaatctaactatgatttaggtcttgccccaccgttacaggatttctacacggtgttgtcatcatcccataaaagagccgttgttcttgctgaagtcaagcgtgcctctccatcgaagggacccatttgtttaaaagctgttgctgctgaacaggctctcaaatacgcagaggctggtgcatccgcaatttccgtattgaccgaacctcattggtttcacggttcgttacaggatttagtaaatgtgaggaaaatcctagatttgaaatttcctcccaaggagaggccttgtgttttgagaaaagaatttattttcagcaagtatcaaatactagaagcaagattagctggagctgacactgtccttcttatagtcaagatgctatctcaacccttattgaaggaactgtacagctacagtaaagatttgaacatggaacctctcgttgaggtgaactccaaagaggaattacaaagggctctagaaattggtgctaaagttgtaggtgtcaataatagggacctgcattcattcaacgtagacctaaataccaccagtaacttggtagaatctattccaaaggatgttcttctaattgctctatcgggaattaccaccagggacgatgctgaaaaatacaaaaaagaaggtgtccatggatttttagtgggtgaagccctaatgaaatcaaccgatgtgaagaagttcattcatgaattatgcgaataa

SEQ ID NO: 18 - DNA, 1143 bp, Saccharomyces cerevisiae
atgtccgaggcgactttgctatcttacaccaagaaattattggcttctccgccgcaattgagtagcacagacctacacgatgcgttgctggttatattaagtcttttgcaaaaatgtgatacaaatagcgatgagagtctttccatctataccaaagtttcgagttttctcacggccttgagagttactaaacttgatcacaaggctgaatacattgcggaagctgcaaaggctgtgctcagacattccgaccttgttgatctacctttacccaagaaggacgaattacacccggaagatggaccagtaatcttagatattgtaggtactggtggtgacggacagaatacttttaatgtttccacgtctgctgctatcgttgcctccggaattcagggcctaaaaatttgtaagcacggtggtaaagcttctacatccaatagtggagctggtgacctaattggaactttaggctgtgacatgttcaaggttaattcatcgacagtgcccaaactttggcctgataatacgttcatgtttctacttgctcctttttttcatcatggaatgggccacgtttctaagatacgcaaatttcttggaattccgactgttttcaacgtactgggaccacttctacatccagttagccacgtaaacaagagaatattgggcgtttactcaaaggaacttgcgcctgaatatgccaaggcagccgctttggtatatccaggaagcgaaacttttatagtttggggacatgttgggttagacgaagtatcacctataggcaaaactactgtctggcatattgatccgacatcgtccgaacttaaattgaagaccttccaattagaaccttctatgtttggtttagaagaacacgagttgtcgaagtgtgcttcatacggccctaaagagaatgcgagaattctaaaagaagaagtcttgtccggcaagtaccaccttggcgacaataatcctatttatgactacatcttgatgaacaccgccgtgttatattgtttaagccaaggtcaccagaactggaaggaagggatcattaaggcagaagaaagcatacattctggtaatgcattacgttctttagaacactttatagatagtgtgagctccttgtag

SEQ ID NO: 19 - DNA, 2124 bp, Saccharomyces cerevisiae
atgtcagaacaactcagacaaacatttgctaacgctaaaaaagaaaacaggaacgccttggtcacatttatgaccgcaggttacccaacagtcaaagacactgtccctattctcaagggtttccaggatggtggtgtagatatcatcgaattgggtatgcccttctctgatccaattgcagatggtcctacaattcaattatctaatactgtggctttgcaaaacggtgttaccttgcctcaaactctagaaatggtctcccaagctagaaatgaaggtgttaccgtacccataatcctaatgggttactataaccctattctaaactacggtgaagaaagatttattcaggacgctgccaaggctggtgctaatggttttatcatcgtcgatttgccaccagaggaggcgttgaaggtcagaaactacatcaatgataatggtttgagcctgatcccactagtggctccttctaccaccgacgaaagattggaattactatcgcatattgccgattcgtttgtctacgttgtgtctagaatgggtactactggtgttcaaagttctgtggccagtgatttggatgaactcatctctagagtcagaaagtacaccaaggatactcctttggccgttgggtttggtgtctctaccagagaacatttccaatcagttggtagtgttgctgacggtgtagtgattggttccaaaatcgtcacattatgtggagatgctccagagggcaaaaggtacgacgttgctaaggaatatgtacagggaattctaaatggtgctaagcataaggttctgtccaaggacgaattctttgcctttcaaaaagagtccttgaagtccgcaaacgttaagaaggaaatactggacgaatttgatgaaaatcacaagcacccaattagatttggggactttggtggtcagtatgtcccagaagctcttcatgcatgtctaagagagttggaaaagggttttgatgaagctgtcgccgatcccacattctgggaagacttcaaatccttgtattcttatattggccgtccttcttcactacacaaagctgagagattaactgagcattgtcaaggtgctcaaatctggttgaagagagaagatcttaaccacacgggatctcacaagatcaacaatgctttagcacaagttcttctagctaaaagattaggcaagaagaacgttattgctgaaaccggtgctggtcaacacggtgttgccactgccactgcatgtgctaaatttggcttaacctgtactgtgttcatgggtgcagaagatgttcgtcgccaagctttaaacgtcttcagaatgagaattctcggtgctaaagtaattgctgttactaatggtacaaagactctaagagacgctacttcagaggcattcagattttgggttactaacttgaaaactacttactacgtcgtcggttctgccattggtcctcacccatatccaactttggttagaactttccaaagtgtcattggtaaagaaaccaaggaacagtttgctgccatgaacaatggtaaattacctgacgcagttgttgcatgtgttgggggtggttccaactctacaggtatgttttcaccattcgagcatgatacttccgttaagttattgggtgtggaagccggtggtgatggtgtagatacaaagttccactctgctactctaactgccggtagacctggtgtcttccatggtgtcaagacttatgtcttgcaagatagtgatggtcaagtccatgatactcattctgtttctgctgggttagactacccaggtgtcggtccagaattggcatattggaaatctactggccgtgctcaattcattgcagctactgacgctcaggctctgcttggctttaaattattatctcaattagaaggtattattcccgctttggaatcttctcatgctgtttatggcgcttgcgaattggctaagacgatgaagcctgatcaacatttggttatcaatatttctggtagaggtgataaagatgtccaaagtgtcgctgaagtcttgccgaaattaggtccaaagataggttgggatttgagattcgaagaagacccatctgcctaa

SEQ ID NO: 20 - DNA, 1188 bp, Saccharomyces cerevisiae
atgtctttggaaagagaggaaccacaacatttcggagcagggccagctcaaatgcctacaccagttttgcaacaagctgctaaagacttaatcaatttcaatgacataggtttgggtatcggtgaaatttctcaccgttcgaaggatgccaccaaagtgattgaagactctaagaagcacttaatcgaactgctaaatattcctgacactcatgaagtgttctacttgcaaggtggtggcactactggtttttcttccgttgctactaatttggcagctgcatatgtgggtaagcatgggaagattgcacctgccggttatttagtcaccggtagttggtctcagaaatcttttgaagaggcaaagagattgcacgttcctgctgaagttatcttcaacgctaaagattataacaatggcaaatttggtaaaattccggatgaatccctttgggaagataaaatcaaaggtaaggctttctcatacgtgtacctatgtgagaatgaaactgttcatggtgttgaatggccagaattaccaaaatgtttagtaaacgaccccaacatcgaaattgttgctgacttatccagcgacattttgtctcgtaagattgacgtttctcaatacggtgtcatcatggcaggcgcccaaaaaaacattggtttagcaggcttaaccctatacattatcaagaaatccatccttaagaatatttctggcgcttctgatgaaacattacatgaattgggagtaccaatcacccctattgcattcgactatccaacggtggtgaagaacaactcagcctataatacaattccaattttcactttacatgttatggatctcgtgttccaacatattttgaagaagggtggtgttgaagcgcaacaggctgaaaatgaagaaaaggccaagatattatatgaggcattggatgcaaattcagatttttacaacgttccagtggatccaaagtgtagatcaaaaatgaatgtcgttttcacccttaaaaaggacggccttgatgaccagtttctaaaagaggcagctgctcgtcatttaaccggtttgaaaggacatcgttcagttggtgggttcagagcctccatctataacgcgctttcagtgaaagctgtacaaaacttggtagattttatcaaggaatttgctgagaaaaacgcttaa

SEQ ID NO: 21 - DNA, 930 bp, Saccharomyces cerevisiae
atgtcaaagtttgttatcacctgcatagctcatggagaaaatctcccaaaagaaaccatcgaccagattgcgaaagaaattactgaaagctcggcgaaagatgtatcaatcaatggtaccaagaaactatcggccagagctaccgatatattcattgaagtcgcaggatcgattgttcaaaaagacctcaagaataagctgacgaacgtcattgacagtcataatgatgttgatgttattgtttctgtcgacaatgaatatcgtcaagccaaaaagctctttgtctttgacatggattcaacattaatctaccaagaggtcatcgaattgattgccgcttatgctggtgttgaagaacaagtgcacgagatcacagaaagagccatgaacaatgaacttgatttcaaagagtctttaagagaacgcgttaaattattgcaaggcctccaagtcgatacactatatgatgaaataaaacaaaagctagaggtcaccaagggtgtgccagaactatgcaagttccttcacaaaaaaaattgcaagctcgctgttttaagcggtggttttattcagtttgccggttttatcaaggatcagttaggtttagatttttgtaaggcaaacttgttggaagttgacactgatggaaaattaaccggtaaaacactgggtcctatcgtagacggacagtgcaagagtgaaacccttctgcaactatgtaatgactacaatgttccagttgaagcaagttgtatggtgggtgacggtggtaacgacctgccagccatggctaccgccgggtttgggatcgcatggaacgccaagccaaaggtgcagaaagctgcaccttgtaagttgaataccaagagcatgactgacattttatacattcttggttacaccgatgatgaaatatacaatagacaatga

SEQ ID NO: 22 - DNA, 1410 bp, Saccharomyces cerevisiae
atgacaagcattgacattaacaacttacaaaatacctttcaacaagctatgaatatgagcggctccccaggcgctgtttgtacttcacctacgcaatctttcatgaataccgttccacagcgcttgaatgctgtaaagcacccaaaaattttgaagcctttctcaacgggtgatatgaagattttactattagaaaacgttaatcaaactgctattacaatcttcgaagagcaaggttaccaagtcgaattctataaatcttcattgcccgaggaagagttgatcgaaaagatcaaggacgttcatgctattggtatcagatcaaagactagattaacttcaaatgtcttacaacatgcgaagaatctggtttgtattggttgtttctgtatcggtaccaaccaagttgacttagactacgctaccagcagaggtattgctgttttcaactcgcctttctccaactcaagatcagtagcagaattggtcatcgctgaaatcattagtttagcaagacaactaggtgatagatctatcgaattacataccggtacatggaataaggttgctgctagatgttgggaggtaagaggaaaaactcttggtattattgggtacggtcacattggttcccaattatcagttcttgcagaagctatgggtttgcatgtgttgtactacgatattgtaactatcatggccttgggtactgccagacaagtttctacattagatgaattgttgaataaatctgattttgtgacactacatgtaccagctactcctgaaactgaaaaaatgttatctgccccacaatttgctgctatgaaggatggcgcttatgttattaatgcttcaagaggtactgtcgtggacattccatctttgatccaagccgtgaaagccaacaaaattgcaggtgctgctttggatgtttatccacatgaaccagctaagaacggtgaaggttcatttaacgatgagctaaatagctggacttctgaattagtttcattaccaaatatcatcttgacaccacacattggtggctctaccgaagaagcccaaagctcaatcggtattgaagtggctaccgcattgtccaaatacatcaatgaaggtaactctgtcggttcagtcaacttcccagaagtggcattgaaatcattgtcttacgaccaagagaacactgtgcgtgtgttatacattcaccaaaatgtaccaggtgttttgaagaccgtcaatgatattttatcgaaccataacatcgaaaagcaattttccgattcaaatggtgaaattgcttatttaatggctgatatctcttctgttgaccaaagcgatattaaagatatttatgaacaactaaatcaaacctctgctaagatctcaattagattgctatattaa

SEQ ID NO: 23 - DNA, 1503 bp, Saccharomyces cerevisiae
atgactttacctgaatcaaaagacttttcttacttgttttcggatgaaaccaatgctcgtaaaccatccccattgaaaacctgcatccatcttttccaagatcctaacattatctttttgggtggtggcctgccattaaaagattatttcccatgggataatctatctgtagattcacccaagcctccttttccccagggtattggagctccaattgacgagcagaattgcataaaatacaccgtcaacaaagattacgctgataaaagtgccaatccttccaacgatattcctttgtcaagagctttgcaatacgggttcagtgctggtcaacctgaactattaaacttcattagagatcataccaagattatccacgatttgaagtataaggactgggacgttttagccactgcaggtaacacaaatgcctgggaatctactttaagagtcttttgtaaccgaggtgatgtcatcttagttgaggcacattctttttcctcttcattggcttctgcagaggctcaaggtgtcattaccttccccgtgccaattgacgctgatggtatcattcctgaaaaattagctaaagtcatggaaaactggacacctggtgctcctaaaccaaagttgttatacactattccaacgggccaaaatccaactggtacttccattgcagaccatagaaaggaggcaatttacaagatcgctcaaaagtacgacttcctaattgtggaagatgaaccttattatttcttacaaatgaatccctacatcaaagacttgaaggaaagagagaaggcacaaagttctccaaagcaggaccatgacgaatttttgaagtccttggcaaacactttcctttccttggatacagaaggccgtgttattagaatggattccttttcaaaagttttggccccagggacaagattgggttggattactggttcatccaaaatcttgaagccttacttgagtttgcatgaaatgacgattcaagccccagcaggttttacacaagttttggtcaacgctacgctatccaggtggggtcaaaagggttacttggactggttgcttggcctgcgtcatgaatacactttgaaacgtgactgtgccatcgatgccctttacaagtatctaccacaatctgatgctttcgtgatcaatcctccaattgcaggtatgtttttcaccgtgaacattgacgcatctgtccaccctgagtttaaaacaaaatacaactcagacccttaccagctagaacagagtctttaccacaaagtggttgaacgtggtgttttagtggttcccggttcttggttcaagagtgagggtgagacggaacctcctcaacccgctgaatctaaagaagtcagtaatccaaacataattttcttcagaggtacctatgcagctgtctctcctgagaaactgactgaaggtctgaagagattaggtgatactttatacgaagaatttggtatttccaaatag

SEQ ID NO: 24 - DNA, 1542 bp, Saccharomyces cerevisiae
atgactgctggttctgccccccctgttgattacacttccttaaagaagaacttccaaccgtttctctccagaagagtagaaaatagatctctgaaaagcttttgggatgcttctgatatctcagatgacgtcattgagctagctggtggaatgccaaacgagagattttttcctatcgaatctatggatttgaaaatatcaaaagttccttttaatgataacccaaaatggcataattcgtttaccacggcgcatttggacttgggatcccccagtgagctacccattgcacgttctttccaatatgcagaaaccaagggtttaccccctctcttacattttgttaaagattttgtgtccagaattaatcgcccagccttttccgatgagacggagtctaactgggatgtcatcctttctggcgggtccaacgattcaatgtttaaggtttttgaaacaatttgcgacgaatcgaccactgtgatgattgaagagtttactttcaccccggctatgtccaatgtggaggctacaggagcaaaagtcatccccatcaagatgaacctgaccttcgacagagagtcccagggtattgatgtcgaatatctaacgcagttgctcgataattggtcaactggaccatacaaagacttaaacaagccaagggtcctatataccattgcaacgggccaaaatcctaccgggatgtctgtcccccagtggaaaagagagaaaatttaccagttggcccaaagacacgatttcctcattgttgaagatgatccctacggttatctgtactttccttcctataatccgcaagagccattagaaaacccttaccattctagcgacctgactactgaacggtatttgaatgattttttaatgaaatcattcttgactttggatacagatgcccgtgtcatccgtttggagactttttctaaaatttttgctcctggattaaggttatccttcatcgttgctaataaattccttttgcaaaaaatcttggatttggccgacattactacaagggcccccagtggtacctcacaagctattgtttattctacaataaaggcaatggctgagtccaacttatcgtcctctctttctatgaaagaagcaatgtttgagggttggataagatggataatgcagattgcttctaaatacaatcataggaaaaatcttactttgaaagccttatacgaaacagaatcttaccaagctggtcagtttaccgttatggaaccctccgcgggtatgttcatcattattaaaatcaattgggggaatttcgatagacctgacgatttgccgcaacagatggatattttagataagttcttgctgaagaatggtgttaaagtagtgcttggttataaaatggctgtttgcccaaattattcaaagcagaattcagattttctaagactcaccatcgcctatgcaagggatgatgatcagttgattgaagcttccaaaagaatcggtagtggcataaaagaattttttgacaactataaaagttga

SEQ ID NO: 25 - DNA, 6438 bp, Saccharomyces cerevisiae
atgccagtgttgaaatcagacaatttcgatccattggaagaagcttacgaaggtgggacaattcaaaactataacgatgaacaccatcttcataaatcttgggcaaatgtgattccggacaaacgaggactttacgaccctgattatgaacatgacgcttgtggtgtcggtttcgtagcaaataagcatggtgaacagtctcacaagattgttactgacgctagatatcttttagtgaatatgacacatcgtggtgccgtctcatctgatgggaacggtgacggtgccggtattctgctaggtattcctcacgaatttatgaaaagagaattcaagttagatcttgatctagacatacctgagatgggcaaatacgccgtaggtaacgtcttcttcaagaagaacgaaaaaaataacaagaaaaatttaattaagtgtcagaagattttcgaggatttagctgcatccttcaacttatccgtattaggttggagaaacgtccccgtagattctactattttaggagacgttgcattatctcgtgaacctactattctacagccattattggttccattgtatgatgaaaaacaaccggagtttaatgaaactaaatttagaactcaattgtatcttttaaggaaggaggcctctcttcaaataggactggaaaactggttctatgtttgttccctaaacaataccaccattgtttacaagggtcaattgacgccagctcaagtgtataactactatcccgacttgactaatgcgcatttcaaatcccacatggcgttggtccattcaagattttccactaatactttcccctcttgggatagagctcaacctttacgttggctagctcataatggtgaaattaacaccttaagaggtaacaagaattggatgcgctccagagaaggtgtgatgaattcagcaactttcaaagatgagttagacaaactatacccaattatcgaagaaggtggttctgattcagctgcattggataacgttttagaactattgactattaatggcacattatctctacctgaagctgttatgatgatggttcctgaagcgtatcataaggatatggattctgacctaaaagcatggtacgactgggctgcatgtctgatggaaccttgggatggtccagctttgttaactttcactgatggacgttactgtggtgctatattggatagaaatggtttaagaccttgtcgttattacatcactagtgatgacagagttatctgtgcttcagaggtaggtgtcattcctatcgaaaattcattggttgttcaaaaaggtaaactgaagccaggtgatttattcctagtggatactcaattgggtgaaatggtcgatactaaaaagttaaaatctcaaatctcaaaaagacaagattttaagtcttggttatccaaagtcatcaagttagacgacttgttatcaaaaaccgctaatttggttcctaaagaatttatatcacaggattcattgtctttgaaagttcaaagtgacccacgtctattggccaatggttataccttcgaacaagtcacatttctgttaactccaatggctttaacaggtaaagaagctttaggttcgatgggtaacgatgcgccactggcttgtttaaatgaaaatcctgtcttactttatgattatttcagacaattgtttgctcaagtgaccaatcctccaattgacccaattcgtgaagcaaatgttatgtcgttagaatgttatgtcggacctcaaggcaaccttttggaaatgcattcatctcaatgtgatcgtttattattgaaatctcctattttgcattggaatgagttccaagctttgaaaaacattgaagctgcttacccatcatggtctgtagcagaaattgatatcacattcgacaagagtgagggtctattgggctataccgacacaattgataaaatcactaagttagcgagcgaagcaattgatgatggtaaaaagatcttaataattactgacaggaaaatgggtgccaaccgtgtttccatctcctctttgattgcaatttcatgtattcatcatcacctaatcagaaacaagcagcgttcccaagttgctttgattttggaaacaggtgaagccagagaaattcaccatttctgtgtcctactaggttatggttgtgatggtgtttatccatacttagccatggaaactttggtcagaatgaatagagaaggtctacttcgtaatgtcaacaatgacaatgatacacttgaggaagggcaaatactagaaaattacaagcacgctattgatgcaggtatcttgaaggttatgtctaaaatgggtatctccactctagcatcctacaaaggtgctcaaatttttgaagccctaggtttagataactctattgttgatttgtgtttcacaggtacttcttccagaattagaggtgtaactttcgagtatttggctcaagatgccttttctttacatgagcgtggttatccatccagacaaaccattagtaaatctgttaacttaccagaaagtggtgaataccactttagggatggtggttacaaacacgtcaacgaaccaaccgcaattgcttcgttacaagatactgtcagaaacaaaaatgatgtctcttggcaattatatgtaaagaaggaaatggaagcaattagagactgtacactaagaggactgttagaattagattttgaaaattctgtcagtatccctctagaacaagttgaaccatggactgaaattgccagaagatttgcgtcaggtgcaatgtcttatggttctatttctatggaagctcactctacattggctattgccatgaatcgtttaggggccaaatccaattgtggtgaaggtggtgaagacgcagaacgttctgctgttcaagaaaacggtgatactatgagatctgctatcaaacaagttgcttccgctagattcggtgtaacttcatactacttgtcagatgctgatgaaatccaaattaagattgctcagggtgctaagccgggtgaaggtggtgaactaccagcccacaaagtgtctaaggatatcgcaaaaaccaggcactccacccctaatgttgggttaatctctcctcctcctcatcacgatatttattccattgaagatttgaaacaactgatttatgatttgaaatgtgctaatccaagagcgggaatttctgtaaagttggtttccgaagttggtgttggtattgttgcctctggtgtagctaaggctaaagccgatcatatcttagtttctggtcatgatggtggtacaggtgctgcaagatggacgagtgtcaaatatgcgggtttgccatgggaattaggtctagctgaaactcaccagactttagtcttgaatgatttaagacgtaatgttgttgtccaaaccgatggtcaattgagaactgggtttgatattgctgttgcagttttattaggggcagaatcttttaccttggcaacagttccattaattgctatgggttgtgttatgttaagaagatgtcacttgaactcttgtgctgttggtattgccacacaagatccatatttgagaagtaagtttaagggtcagcccgaacatgttatcaacttcttctattacttgatccaagatttaagacaaatcatggccaagttaggattccgtaccattgacgaaatggtgggtcattctgaaaaattaaagaaaagggacgacgtaaatgccaaagccataaatatcgatttatctcctattttgaccccagcacatgttattcgtccaggtgttccaaccaagttcactaagaaacaagaccacaaactccacacccgtctagataataagttaatcgatgaggctgaagttactttggatcgtggcttaccagtgaatattgacgcctctataatcaatactgatcgtgcactcggttctactttatcttacagagtctcgaagaaatttggtgaagatggtttgccaaaggacaccgttgtcgttaacatagaaggttcagcgggtcaatcttttggtgctttcctagcttctggtatcacttttatcttgaatggtgatgctaatgattatgttggtaaaggtttatccggtggtattattgtcattaaaccaccaaaggattctaaattcaagagtgatgaaaatgtaattgttggtaacacttgtttctatggtgctacttctggtactgcattcatttcaggtagtgccggtgagcgtttcggtgtcagaaactctggtgccaccatcgttgttgagagaattaagggtaacaatgcctttgagtatatgactggtggtcgtgccattgtcttatcacaaatggaatccctaaacgccttctctggtgctactggtggtattgcatactgtttaacttccgattacgacgattttgttggaaagattaacaaagatactgttgagttagaatcattatgtgacccggtcgagattgcgtttgttaagaatttgatccaggagcattggaactacacacaatctgatctagcagccaggattctcggtaatttcaaccattatttgaaagatttcgttaaagtcattccaactgattataagaaagttttgttgaaggagaaagcagaagctgccaaggcaaaggctaaggcaacttcagaatacttaaagaagtttagatcgaaccaagaagttgatgacgaagtcaatactctattgattgctaatcaaaaagctaaagagcaagaaaaaaagaagagtattactatttcaaataaggccactttgaaggagcctaaggttgttgatttagaagatgcagttccagattccaaacagctagagaagaatagcgaaaggattgaaaaaacacgtggttttatgatccacaaacgtcgtcatgagacacacagagatccaagaaccagagttaatgactggaaagaatttactaaccctattaccaagaaggatgccaaatatcaaactgcgagatgtatggattgtggtacaccattctgtttatctgataccggttgtcccctatctaacattatccccaagtttaatgaattgttattcaagaaccaatggaagttggcactggacaaattgctagagacaaacaatttcccagaattcactggaagagtatgtccagcaccctgtgagggagcttgtacactaggtattattgaagacccagtcggcataaaatcggttgaaagaattatcattgacaatgctttcaaggaaggatggattaagccttgtccaccaagtacacgcactggctttacagtgggtgtcattggttctggtccagcaggtttagcgtgtgctgatatgttgaaccgtgccggacatacggtcactgtttatgaaagatccgaccgttgtggtgggttattgatgtatggtattccaaacatgaagttggataaggctatagtgcaacgtcgtattgatctattgagtgccgaaggtattgactttgttaccaacaccgaaattggtaaaaccataagcatggatgagctaaagaacaagcacaatgcagtagtgtatgctatcggttctaccattccacgtgacttacctattaagggtcgtgaattgaagaatattgattttgccatgcagttgttggaatctaacacaaaagctttattgaacaaagatctggaaatcattcgtgaaaagatccaaggtaagaaagtaattgttgtcggtggtggtgacacaggtaacgattgtttaggtacatctgtaagacacggtgcagcatcagttttgaatttcgaattgttgcctgagccaccagtggaacgtgccaaagacaatccatggcctcaatggccgcgtgtcatgagagtggactacggtcatgctgaagtgaaagagcattatggtagagaccctcgtgaatactgcatcttgtccaaggaatttatcggtaacgatgagggtgaagtcactgccatcagaactgtgcgcgtagaatggaagaagtcacaaagtggcgtatggcaaatggtagaaattcccaacagtgaagagatctttgaagccgatatcattttgttgtctatgggtttcgtgggtcctgaattgatcaatggcaacgataacgaagttaagaagacaagacgtggtacgattgccacactcgacgactcctcatactctattgatggaggaaagacttttgcatgtggtgactgtagaagagggcaatctttgattgtctgggccatccaagaaggtagaaaatgtgctgcctctgtcgataagttcctaatggacggcactacgtatctaccaagtaatggtggtatcgttcaacgtgattacaaactattgaaagaattagctagtcaagtctaa

SEQ ID NO: 26 - DNA, 1503 bp, Saccharomyces cerevisiae
atgtctagattagaaagattgacctcattaaacgttgttgctggttctgacttgagaagaacctccatcattggtaccatcggtccaaagaccaacaacccagaaaccttggttgctttgagaaaggctggtttgaacattgtccgtatgaacttctctcacggttcttacgaataccacaagtctgtcattgacaacgccagaaagtccgaagaattgtacccaggtagaccattggccattgctttggacaccaagggtccagaaatcagaactggtaccaccaccaacgatgttgactacccaatcccaccaaaccacgaaatgatcttcaccaccgatgacaagtacgctaaggcttgtgacgacaagatcatgtacgttgactacaagaacatcaccaaggtcatctccgctggtagaatcatctacgttgatgatggtgttttgtctttccaagttttggaagtcgttgacgacaagactttgaaggtcaaggctttgaacgccggtaagatctgttcccacaagggtgtcaacttaccaggtaccgatgtcgatttgccagctttgtctgaaaaggacaaggaagatttgagattcggtgtcaagaacggtgtccacatggtcttcgcttctttcatcagaaccgccaacgatgttttgaccatcagagaagtcttgggtgaacaaggtaaggacgtcaagatcattgtcaagattgaaaaccaacaaggtgttaacaacttcgacgaaatcttgaaggtcactgacggtgttatggttgccagaggtgacttgggtattgaaatcccagccccagaagtcttggctgtccaaaagaaattgattgctaagtctaacttggctggtaagccagttatctgtgctacccaaatgttggaatccatgacttacaacccaagaccaaccagagctgaagtttccgatgtcggtaacgctatcttggatggtgctgactgtgttatgttgtctggtgaaaccgccaagggtaactacccaatcaacgccgttaccactatggctgaaaccgctgtcattgctgaacaagctatcgcttacttgccaaactacgatgacatgagaaactgtactccaaagccaacctccaccaccgaaaccgtcgctgcctccgctgtcgctgctgttttcgaacaaaaggccaaggctatcattgtcttgtccacttccggtaccaccccaagattggtttccaagtacagaccaaactgtccaatcatcttggttaccagatgcccaagagctgctagattctctcacttgtacagaggtgtcttcccattcgttttcgaaaaggaacctgtctctgactggactgatgatgttgaagcccgtatcaacttcggtattgaaaaggctaaggaattcggtatcttgaagaagggtgacacttacgtttccatccaaggtttcaaggccggtgctggtcactccaacactttgcaagtctctaccgtttaa

SEQ ID NO: 27 - DNA, 2232 bp, Saccharomyces cerevisiae
atggatggtcccaattttgcacatcaaggcgggagatcacaacgtactactgaattgtattcgtgcgcacgatgcagaaaattaaagaagaagtgtggtaaacaaataccgacatgtgcaaactgcgataaaaatggggcacactgttcatatccaggtagagccccaagacgtaccaagaaggagttagcggatgctatgctacgaggggaatatgttccagtgaaaaggaacaagaaggtaggaaaaagcccattgagcactaagagcatgccaaactcttctagtccgctatccgcaaatggcgctataactcccgggttttcgccttacgaaaacgatgatgcacataagatgaaacagctaaaaccgtcagatccaataaatcttgtcatgggggcaagtccaaattctagcgaaggtgtctcatcgctaatttcggtgctaacatcgctgaatgataattctaatccttcttcgcacttatcctctaatgaaaattccatgattccttcccgatcattgccagcttccgtgcagcaaagttcgacaacttcatcattcggaggatataacacgccttcaccactaattagcagtcatgtgcctgcgaacgcccaagccgtaccgctacaaaacaacaatcgcaatactagcaacggggataacggcagtaatgttaaccacgataataacaatggcagtaccaacacaccgcaattgagtcttaccccatatgcaaacaattcagcccccaatgggaaattcgattctgtgccggttgatgcatcctcgatcgaatttgaaactatgtcctgttgctttaaaggtggtagaacaacatcgtgggtcagagaggatggctcgttcaagtcaattgatagatccttactggacaggttcattgccgcatacttcaaacacaatcaccgtctatttcccatgattgataaaatagcattcctaaatgacgccgcgacaattactgatttcgaaaggttatatgacaacaaaaactaccctgacagctttgttttcaaagtatacatgatcatggctattggttgtacaactttacagcgtgctggtatggtttctcaggacgaagagtgtctgagtgaacatttggcttttttggccatgaaaaaatttcgtagtgttataattttacaagatatcgaaactgtacgatgcctattgttgttgggtatttattcgttttttgagccaaagggctcctcgtcatggacaattagtggtatcatcatgcgattgaccataggattaggtttaaatagagagttaactgccaaaaaactcaagagcatgtctgctttagaagcagaggcaagatatagagtgttttggagtgcttactgctttgaaaggctagtatgcacctcgttgggccgtatatccgggatcgacgacgaagacatcactgtgccactaccgagggcgttgtatgtggatgaaagagacgatttagagatgaccaagttgatgatatcattaaggaaaatgggcggtcgcatttataaacaagtccactctgtaagtgcagggcgacaaaagttaaccatcgaacaaaagcaggaaatcataagtggattacgcaaggagctagacgaaatttattctcgagaatcagaaagaaggaaactgaaaaaatctcaaatggatcaggtggagagggagaacaattctactacaaatgtaatatccttccatagttctgagatttggctagcaatgagatactcccagttgcaaatcttactatacagaccatctgcattgatgccaaaaccgcccattgactcactatccactctaggagagttttgcttgcaagcctggaaacatacttatacactgtacaagaagcggttattacccttgaactggataacccttttcagaacattaaccatttgtaacactatcttatactgtctttgccagtggagcatcgacctcattgaaagtaaaatcgaaattcaacagtgtgtggaaatactaagacatttcggtgaaagatggatttttgctatgagatgcgcggatgttttccaaaacattagcaacacaattctcgatattagtttaagccatggtaaagttcctaatatggaccaattaacaagagagttatttggcgccagcgattcatatcaagacatattagacgaaaacaatgttgacgtctcttgggttgataaacttgtatga

SEQ ID NO: 28 - DNA, 1245 bp, Saccharomyces cerevisiae
atgtttgtcagggttaaattgaataaaccagtaaaatggtataggttctatagtacgttggattcacattccctaaagttacagagcggctcgaagtttgtaaaaataaagccagtaaataacttgaggagtagttcatcagcagatttcgtgtccccaccaaattccaaattacaatctttaatctggcagaaccctttacaaaatgtttatataactaaaaaaccatggactccatccacaagagaagcgatggttgaattcataactcatttacatgagtcataccccgaggtgaacgtcattgttcaacccgatgtggcagaagaaatttcccaggatttcaaatctcctttggagaatgatcccaaccgacctcatatactttatactggtcctgaacaagatatcgtaaacagaacagacttattggtgacattgggaggtgatgggactattttacacggcgtatcaatgttcggaaatacgcaagttcctccggttttagcatttgctctgggcactctgggctttctatcaccgtttgattttaaggagcataaaaaggtctttcaggaagtaatcagctctagagccaaatgtttgcatagaacacggctagaatgtcatttgaaaaaaaaggatagcaactcatctattgtgacccatgctatgaatgacatattcttacataggggtaattcccctcatctcactaacctggacattttcattgatggggaatttttgacaagaacgacagcagatggtgttgcattggccactccaacgggttccacagcatattcattatcagcaggtggatctattgtttccccattagtccctgctattttaatgacaccaatttgtcctcgctctttgtcattccgaccactgattttgcctcattcatcccacattaggataaagataggttccaaattgaaccaaaaaccagtcaacagtgtggtaaaactttctgttgatggtattcctcaacaggatttagatgttggtgatgaaatttatgttataaatgaggtcggcactatatacatagatggtactcagcttccgacgacaagaaaaactgaaaatgactttaataattcaaaaaagcctaaaaggtcagggatttattgtgtcgccaagaccgagaatgactggattagaggaatcaatgaacttttaggattcaattctagctttaggctgaccaagagacagactgataatgattaa

SEQ ID NO: 29 - DNA, 1518 bp, Saccharomyces cerevisiae
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 30 - DNA, 624 bp, Bos taurus
atgagcaccccgagcattcattgtctgaaaccgagtccgctgcatctgccgagcggtattccgggtagtccgggtcgtcagcgtcgccataccctgcctgcaaatgaatttcgttgtctgacaccggaagatgcagccggtgtttttgaaattgaacgtgaagcatttattagcgtgagcggtaattgtccgctgaacctggatgaagttcgtcattttctgaccctgtgtccggaactgagcctgggttggtttgttgaaggtcgtctggttgcatttatcattggtagcctgtgggatgaagaacgtctgacccaagaaagcctgaccctgcatcgtccgggtggtcgtaccgcacatctgcatgcactggcagttcatcattcatttcgtcagcagggcaaaggtagcgttctgctgtggcgttatctgcaacatgccggtggtcagcctgccgttcgtcgtgcagttctgatgtgtgaagatgcactggttccgttttatcagcgttttggttttcatccggcaggtccgtgtgcagttgttgtgggtagcctgacctttaccgaaatgcattgtagcctgcgtggtcatgcagcactgcgtcgtaatagcgatcgttaa

SEQ ID NO: 31 - DNA, 1410 bp, Saccharomyces cerevisiae
atgtcttattcagctgccgataatttacaagattcattccaacgtgccatgaacttttctggctctcctggtgcagtctcaacctcaccaactcagtcatttatgaacacactacctcgtcgtgtaagcattacaaagcaaccaaaggctttaaaacctttttctactggtgacatgaatattctactgttggaaaatgtcaatgcaactgcaatcaaaatcttcaaggatcagggttaccaagtagagttccacaagtcttctctacctgaggatgaattgattgaaaaaatcaaagacgtacacgctatcggtataagatccaaaactagattgactgaaaaaatactacagcatgccaggaatctagtttgtattggttgtttttgcataggtaccaatcaagtagacctaaaatatgccgctagtaaaggtattgctgttttcaattcgccattctccaattcaagatccgtagcagaattggtaattggtgagatcattagtttagcaagacaattaggtgatagatccattgaactgcatacaggtacatggaataaagtcgctgctaggtgttgggaagtaagaggaaaaactctcggtattattgggtatggtcacattggttcgcaattatcagttcttgcagaagctatgggcctgcatgtgctatactatgatatcgtgacaattatggccttaggtactgccagacaagtttctacattagatgaattgttgaataaatctgattttgtaacactacatgtaccagctactccagaaactgaaaaaatgttatctgctccacaattcgctgctatgaaggacggggcttatgttattaatgcctcaagaggtactgtcgtggacattccatctctgatccaagccgtcaaggccaacaaaattgcaggtgctgctttagatgtttatccacatgaaccagctaagaacggtgaaggttcatttaacgatgaacttaacagctggacttctgagttggtttcattaccaaatataatcctgacaccacatattggtggctctacagaagaagctcaaagttcaatcggtattgaggtggctactgcattgtccaaatacatcaatgaaggtaactctgtcggttctgtgaacttcccagaagtcagtttgaagtctttggactacgatcaagagaacacagtacgtgtcttgtatattcatcgtaacgttcctggtgttttgaagaccgttaatgatatcttatccgatcataatatcgagaaacagttttctgattctcacggcgagatcgcttatctaatggcagacatctcttctgttaatcaaagtgaaatcaaggatatatatgaaaagttgaaccaaacttctgccaaagtttccatcaggttattatactaa

SEQ ID NO: 32 - protein, 511 aa, Streptomyces rugosporus
MIRSVVIVGGGTAGWMTASYLKAAFDDRIDVTLVESGNVRRIGVGEATFSTVRHFFDYLGLDEREWLPRCAGGYKLGIRFENWSEPGEYFYHPFERLRVVDGFNMAEWWLAVGDRRTSFSEACYLTHRLCEAKRAPRMLDGSLFASQVDESLGRSTLAEQRAQFPYAYHFDADEVARYLSEYAIARGVRHVVDDVQHVGQDERGWISGVHTKQHGEISGDLFVDCTGFRGLLINQTLGGRFQSFSDVLPNNRAVALRVPRENDEDMRPYTTATAMSAGWMWTIPLFKRDGNGYVYSDEFISPEEAERELRSTVAPGRDDLEANHIQMRIGRNERTWINNCVAVGLSAAFVEPLESTGIFFIQHAIEQLVKHFPGERWDPVLISAYNERMAHMVDGVKEFLVLHYKGAQREDTPYWKAAKTRAMPDGLARKLELSASHLLDEQTIYPYYHGFETYSWITMNLGLGIVPERPRPALLHMDPAPALAEFERLRREGDELIAALPSCYEYLASIQ

SEQ ID NO: 33 - protein, 523 aa, Streptomyces toxytricini
MNTRNPDKVVIVGGGTAGWMTASYLKKAFGERVSVTLVESGTIGTVGVGEATFSDIRHFFEFLDLREEEWMPACNATYKLAVRFQDWQRPGHHFYHPFEQMRSVDGFPLTDWWLQNGPTDRFDRDCFVMASLCDAGRSPRYLNGSLLQQEFDERAEEPAGLTMSEHQGKTQFPYAYHFEAALLAEFLSGYSKDRGVKHVVDEVLEVKLDDRGWISHVVTKEHGDIGGDLFVDCTGFRGVLLNQALGVPFVSYQDTLPNDSAVALQVPLDMEARGIPPYTRATAKEAGWIWTIPLIGRIGTGYVYAKDYCSPEEAERTLREFVGPEAADVEANHIRMRIGRSEQSWKNNCVAIGLSSGFVEPLESTGIFFIHHAIEQLVKHFPAGDWHPQLRAGYNSAVANVMDGVREFLVLHYLGAARNDTRYWKDTKTRAVPDALAERIERWKVQLPDSENVFPYYHGLPPYSYMAILLGTGAIGLRPSPALALADPAAAEKEFTAIRDRARFLVDTLPSQYEYFAAMGQRV

SEQ ID NO: 34 - protein, 530 aa, Lechenvalieria aerocolonigenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 35 - protein, 170 aa, Lechevalieria aerocolonigenes
MTIEFDRPGAHVTAADHRALMSLFPTGVAVITAIDEAGTPHGMTCTSLTSVTLDPPTLLVCLNRASGTLHAVRGGRFGVNLLHARGRRAAEVFSTAVQDRFGEVRWEHSDVTGMPWLAEDAHAFAGCVVRKSTVVGDHEIVLGEVHEVVREHDLPLLYGMREFAVWTPEG

SEQ ID NO: 36 - protein, 263 aa, Oryctolagus cuniculus
MEGGFTGGDEYQKHFLPRDYLNTYYSFQSGPSPEAEMLKFNLECLHKTFGPGGLQGDTLIDIGSGPTIYQVLAACESFKDITLSDFTDRNREELAKWLKKEPGAYDWTPALKFACELEGNSGRWQEKAEKLRATVKRVLKCDANLSNPLTPVVLPPADCVLTLLAMECACCSLDAYRAALRNLASLLKPGGHLVTTVTLQLSSYMVGEREFSCVALEKEEVEQAVLDAGFDIEQLLYSPQSYSASTAPNRGVCFLVARKKPGS

SEQ ID NO: 37 - DNA, 1536 bp, Streptomyces rugosporus
atgatcagatctgttgttatcgttggtggtggtactgctggttggatgactgcttcttacttgaaggctgctttcgacgacagaatcgacgttactttggttgaatctggtaacgttagaagaatcggtgttggtgaagctactttctctactgttagacacttcttcgactacttgggtttggacgaaagagaatggttgccaagatgtgctggtggttacaagttgggtatcagattcgaaaactggtctgaaccaggtgaatacttctaccacccattcgaaagattgagagttgttgacggtttcaacatggctgaatggtggttggctgttggtgacagaagaacttctttctctgaagcttgttacttgactcacagattgtgtgaagctaagagagctccaagaatgttggacggttctttgttcgcttctcaagttgacgaatctttgggtagatctactttggctgaacaaagagctcaattcccatacgcttaccacttcgacgctgacgaagttgctagatacttgtctgaatacgctatcgctagaggtgttagacacgttgttgacgacgttcaacacgttggtcaagacgaaagaggttggatctctggtgttcacactaagcaacacggtgaaatctctggtgacttgttcgttgactgtactggtttcagaggtttgttgatcaaccaaactttgggtggtagattccaatctttctctgacgttttgccaaacaacagagctgttgctttgagagttccaagagaaaacgacgaagacatgagaccatacactactgctactgctatgtctgctggttggatgtggactatcccattgttcaagagagacggtaacggttacgtttactctgacgaattcatctctccagaagaagctgaaagagaattgagatctactgttgctccaggtagagacgacttggaagctaaccacatccaaatgagaatcggtagaaacgaaagaacttggatcaacaactgtgttgctgttggtttgtctgctgctttcgttgaaccattggaatctactggtatcttcttcatccaacacgctatcgaacaattggttaagcacttcccaggtgaaagatgggacccagttttgatctctgcttacaacgaaagaatggctcacatggttgacggtgttaaggaattcttggttttgcactacaagggtgctcaaagagaagacactccatactggaaggctgctaagactagagctatgccagacggtttggctagaaagttggaattgtctgcttctcacttgttggacgaacaaactatctacccatactaccacggtttcgaaacttactcttggatcactatgaacttgggtttgggtatcgttccagaaagaccaagaccagctttgttgcacatggacccagctccagctttggctgaattcgaaagattgagaagagaaggtgacgaattgatcgctgctttgccatcttgttacgaatacttggcttctatccaatag

SEQ ID NO: 38 - DNA, 1572 bp, Streptomyces toxytricini
atgaacactagaaacccagacaaggttgttatcgttggtggtggtactgctggttggatgactgcttcttacttgaagaaggctttcggtgaaagagtttctgttactttggttgaatctggtactatcggtactgttggtgttggtgaagctactttctctgacatcagacacttcttcgaattcttggacttgagagaagaagaatggatgccagcttgtaacgctacttacaagttggctgttagattccaagactggcaaagaccaggtcaccacttctaccacccattcgaacaaatgagatctgttgacggtttcccattgactgactggtggttgcaaaacggtccaactgacagattcgacagagactgtttcgttatggcttctttgtgtgacgctggtagatctccaagatacttgaacggttctttgttgcaacaagaattcgacgaaagagctgaagaaccagctggtttgactatgtctgaacaccaaggtaagactcaattcccatacgcttaccacttcgaagctgctttgttggctgaattcttgtctggttactctaaggacagaggtgttaagcacgttgttgacgaagttttggaagttaagttggacgacagaggttggatctctcacgttgttactaaggaacacggtgacatcggtggtgacttgttcgttgactgtactggtttcagaggtgttttgttgaaccaagctttgggtgttccattcgtttcttaccaagacactttgccaaacgactctgctgttgctttgcaagttccattggacatggaagctagaggtatcccaccatacactagagctactgctaaggaagctggttggatctggactatcccattgatcggtagaatcggtactggttacgtttacgctaaggactactgttctccagaagaagctgaaagaactttgagagaattcgttggtccagaagctgctgacgttgaagctaaccacatcagaatgagaatcggtagatctgaacaatcttggaagaacaactgtgttgctatcggtttgtcttctggtttcgttgaaccattggaatctactggtatcttcttcatccaccacgctatcgaacaattggttaagcacttcccagctggtgactggcacccacaattgagagctggttacaactctgctgttgctaacgttatggacggtgttagagaattcttggttttgcactacttgggtgctgctagaaacgacactagatactggaaggacactaagactagagctgttccagacgctttggctgaaagaatcgaaagatggaaggttcaattgccagactctgaaaacgttttcccatactaccacggtttgccaccatactcttacatggctatcttgttgggtactggtgctatcggtttgagaccatctccagctttggctttggctgacccagctgctgctgaaaaggaattcactgctatcagagacagagctagattcttggttgacactttgccatctcaatacgaatacttcgctgctatgggtcaaagagtttag

SEQ ID NO: 39 - DNA, 1593 bp, Lechevalieria aerocolonigenes
atgtctggtaagatcgacaagatcttgatcgttggtggtggtactgctggttggatggctgcttcttacttgggtaaggctttgcaaggtactgctgacatcactttgttgcaagctccagacatcccaactttgggtgttggtgaagctactatcccaaacttgcaaactgctttcttcgacttcttgggtatcccagaagacgaatggatgagagaatgtaacgcttcttacaaggttgctatcaagttcatcaactggagaactgctggtgaaggtacttctgaagctagagaattggacggtggtccagaccacttctaccactctttcggtttgttgaagtaccacgaacaaatcccattgtctcactactggttcgacagatcttacagaggtaagactgttgaaccattcgactacgcttgttacaaggaaccagttatcttggacgctaacagatctccaagaagattggacggttctaaggttactaactacgcttggcacttcgacgctcacttggttgctgacttcttgagaagattcgctactgaaaagttgggtgttagacacgttgaagacagagttgaacacgttcaaagagacgctaacggtaacatcgaatctgttagaactgctactggtagagttttcgacgctgacttgttcgttgactgttctggtttcagaggtttgttgatcaacaaggctatggaagaaccattcttggacatgtctgaccacttgttgaacgactctgctgttgctactcaagttccacacgacgacgacgctaacggtgttgaaccattcacttctgctatcgctatgaagtctggttggacttggaagatcccaatgttgggtagattcggtactggttacgtttactcttctagattcgctactgaagacgaagctgttagagaattctgtgaaatgtggcacttggacccagaaactcaaccattgaacagaatcagattcagagttggtagaaacagaagagcttgggttggtaactgtgtttctatcggtacttcttcttgtttcgttgaaccattggaatctactggtatctacttcgtttacgctgctttgtaccaattggttaagcacttcccagacaagtctttgaacccagttttgactgctagattcaacagagaaatcgaaactatgttcgacgacactagagacttcatccaagctcacttctacttctctccaagaactgacactccattctggagagctaacaaggaattgagattggctgacggtatgcaagaaaagatcgacatgtacagagctggtatggctatcaacgctccagcttctgacgacgctcaattgtactacggtaacttcgaagaagaattcagaaacttctggaacaactctaactactactgtgttttggctggtttgggtttggttccagacgctccatctccaagattggctcacatgccacaagctactgaatctgttgacgaagttttcggtgctgttaaggacagacaaagaaacttgttggaaactttgccatctttgcacgaattcttgagacaacaacacggtagatag

SEQ ID NO: 40 - DNA, 513 bp, Lechevalieria aerocolonigenes
atgactatcgaattcgacagaccaggtgctcacgttactgctgctgaccacagagctttgatgtctttgttcccaactggtgttgctgttatcactgctatcgacgaagctggtactccacacggtatgacttgtacttctttgacttctgttactttggacccaccaactttgttggtttgtttgaacagagcttctggtactttgcacgctgttagaggtggtagattcggtgttaacttgttgcacgctagaggtagaagagctgctgaagttttctctactgctgttcaagacagattcggtgaagttagatgggaacactctgacgttactggtatgccatggttggctgaagacgctcacgctttcgctggttgtgttgttagaaagtctactgttgttggtgaccacgaaatcgttttgggtgaagttcacgaagttgttagagaacacgacttgccattgttgtacggtatgagagaattcgctgtttggactccagaaggttag

SEQ ID NO: 41 - DNA, 792 bp, Oryctolagus cuniculus
atggaaggtggtttcactggtggtgacgaataccaaaagcacttcttgccaagagactacttgaacacttactactctttccaatctggtccatctccagaagctgaaatgttgaagttcaacttggaatgtttgcacaagactttcggtccaggtggtttgcaaggtgacactttgatcgacatcggttctggtccaactatctaccaagttttggctgcttgtgaatctttcaaggacatcactttgtctgacttcactgacagaaacagagaagaattggctaagtggttgaagaaggaaccaggtgcttacgactggactccagctttgaagttcgcttgtgaattggaaggtaactctggtagatggcaagaaaaggctgaaaagttgagagctactgttaagagagttttgaagtgtgacgctaacttgtctaacccattgactccagttgttttgccaccagctgactgtgttttgactttgttggctatggaatgtgcttgttgttctttggacgcttacagagctgctttgagaaacttggcttctttgttgaagccaggtggtcacttggttactactgttactttgcaattgtcttcttacatggttggtgaaagagaattctcttgtgttgctttggaaaaggaagaagttgaacaagctgttttggacgctggtttcgacatcgaacaattgttgtactctccacaatcttactctgcttctactgctccaaacagaggtgtttgtttcttggttgctagaaagaagccaggttcttaa

SEQ ID NO: 42 - protein, 125 aa, Psilocybe cubensis
MSADKIVTLEELRAHKSRDKLYILIHGKVYNVTKFLDEHPGGDEVLLAEGGMDATEAFEDVGHSDEARALLPGMLVGSFEEGALKSGAASAHGAKVATAVEQGSNLMYFVPLGLLGAYFAWQYYS

SEQ ID NO: 43 - DNA, 378 bp, Psilocybe cubensis
atgtctgctgacaagatcgttactttggaagaattgagagctcacaagtctagagacaagttgtacatcttgatccacggtaaggtttacaacgttactaagttcttggacgaacacccaggtggtgacgaagttttgttggctgaaggtggtatggacgctactgaagctttcgaagacgttggtcactctgacgaagctagagctttgttgccaggtatgttggttggttctttcgaagaaggtgctttgaagtctggtgctgcttctgctcacggtgctaaggttgctactgctgttgaacaaggttctaacttgatgtacttcgttccattgggtttgttgggtgcttacttcgcttggcaatactactcttaa

SEQ ID NO: 44 - protein, 473 aa, Saccharomyces cerevisiae
MAKDNSEKLQVQGEEKKSKQPVNFLPQGKWLKPNEIEYEFGGTTGVIGMLIGFPLLMYYMWICAEFYHGKVALPKAGESWMHFIKHLYQLVLENGIPEKYDWTIFLTFWVFQIIFYYTLPGIWTKGQPLSHLKGKQLPYFCNAMWTLYVTTTLVLVLHFTNLFRLYVIIDRFGRIMTCAIISGFAFSIILYLWTLFISHDYHRMTGNHLYDFFMGAPLNPRWGILDLKMFFEVRLPWFTLYFITLGACLKQWETYGYVTPQLGVVMLAHWLYANACAKGEELIVPTWDMAYEKFGFMLIFWNIAGVPYTYCHCTLYLYYHDPSEYHWSTLYNVSLYVVLLCAYYFFDTANAQKNAFRKQMSGDKTGRKTFPFLPYQILKNPKYMVTSNGSYLLIDGWYTLARKIHYTADWTQSLVWALSCGFNSVFPWFFPVFFLVVLIHRAFRDQAKCKRKYGKDWDEYCKHCPYVFIPYVF

SEQ ID NO: 45 - DNA, 1422 bp, Saccharomyces cerevisiae
atggcaaaggataatagtgagaagctgcaggtgcagggagaggagaaaaagtccaagcaaccggttaatttcctgcctcagggtaaatggctgaagccaaatgaaatcgaatatgagtttggtgggactactggtgttattggtatgctgatcgggtttccactgctaatgtactatatgtggatttgtgcggaattttatcacggtaaggttgccctacccaaggctggtgaatcgtggatgcactttatcaagcacctataccagttagtcttggagaacggtatcccagaaaagtatgactggactattttcttaacattttgggtgtttcagatcattttctactatacgttgcccgggatttggacaaaaggtcaaccattgtctcatttgaagggaaaacaattgccttacttttgtaatgccatgtggaccttgtatgtaactaccactttggtcttggttttgcactttaccaatctttttagattgtatgtcattattgaccgttttgggaggatcatgacatgtgccattatttcagggtttgccttctccatcatattgtacttatggactttatttatctcacatgactatcatagaatgacaggaaaccatctatatgatttcttcatgggagctccactaaaccctaggtgggggattttggacttgaagatgtttttcgaggttagattaccttggttcaccctttactttatcactttgggtgcctgtttgaagcagtgggagacttacggctatgtgacaccacaattgggggttgtcatgttagctcattggttgtacgcgaacgcatgtgctaaaggtgaagaattgattgttccaacctgggacatggcttacgaaaagtttggatttatgctgatcttctggaatattgccggtgtcccatacacttactgtcattgtacgttgtatttgtactaccatgacccatctgaatatcactggtctacactgtacaatgtttcgctgtacgttgttctattatgcgcctactacttctttgacacggcaaatgctcagaaaaatgccttcagaaagcaaatgtctggtgacaagacaggtaggaagactttcccatttttgccataccaaattttgaagaatccaaagtatatggttacctccaatggatcgtacctattgattgatggttggtacactttggctagaaaaattcactacactgccgattggactcaatctctcgtttgggccttgtcttgcgggttcaactcggtgttcccatggtttttcccagtattcttccttgttgtcctgattcacagagccttcagagaccaagcaaaatgtaagagaaagtacggaaaagattgggatgagtattgtaaacattgcccttacgtctttattccttatgttttctag

SEQ ID NO: 46 - protein, 396 aa, Saccharomyces cerevisiae
MTVTIKELTNHNYIDHELSATLDSTDAFEGPEKLLEIWFFPHKKSITTEKTLRNIGMDRWIEILKLVKCEVLSMKKTKELDAFLLSESSLFVFDHKLTMKTCGTTTTLFCLEKLFQIVEQELSWAFRTTQGGKYKPFKVFYSRRCFLFPCKQAAIHQNWADEVDYLNKFFDNGKSYSVGRNDKSNHWNLYVTETDRSTPKGKEYIEDDDETFEVLMTELDPECASKFVCGPEASTTALVEPNEDKGHNLGYQMTKNTRLDEIYVNSAQDSDLSFHHDAFAFTPCGYSSNMILAEKYYYTLHVTPEKGWSYASFESNIPVFDISQGKQDNLDVLLHILNVFQPREFSMTFFTKNYQNQSFQKLLSINESLPDYIKLDKIVYDLDDYHLFYMKLQKKI

SEQ ID NO: 47 - DNA, 1191 bp, Saccharomyces cerevisiae
atgactgtcaccataaaagaattgactaaccacaactacattgaccacgaactatcagccactttagactcaacggatgcgttcgagggtcccgagaagttgctggaaatctggttcttccctcacaagaagtccatcacgaccgaaaagacattaagaaatattggcatggatagatggatcgagattttgaaattagtgaaatgcgaagttctttccatgaagaagactaaagaactggatgcctttttgttgagtgagtcttccctcttcgtcttcgatcacaaattgacgatgaagacgtgcggtactacaaccacattgttctgtctcgaaaagcttttccagatcgttgagcaagagttatcgtgggctttccgcacaacacaagggggcaagtacaaaccatttaaagtgttttattctagacgatgtttccttttcccctgtaagcaagccgctatccatcaaaactgggctgacgaagtcgactatttgaacaaatttttcgacaatggtaaaagttattccgtgggaagaaatgacaagagcaaccactggaacctgtacgtcaccgagacggaccgctccacacctaagggaaaggagtacatcgaggatgacgacgaaactttcgaagtactgatgacggagctggacccagaatgcgctagtaagtttgtttgcgggcctgaggcatccacaaccgctctcgtggagccaaacgaagataagggccacaacctcggctaccaaatgactaaaaatacaaggcttgacgaaatatatgtcaactcggcccaagactccgatttatcatttcaccacgatgcatttgcgttcacgccatgtggatactcatccaatatgattctcgctgaaaaatactattacaccctgcacgtgactccggaaaagggttggtcttacgcctctttcgaaagtaacatacccgtatttgacatttcccaagggaagcaagacaacttggacgttcttctacatattctgaacgtttttcaaccaagagagttctcgatgaccttttttaccaaaaattatcagaaccaatccttccaaaaactactaagcatcaacgagtcactgcccgactacatcaagttagacaaaattgtttatgatctggacgactaccaccttttctatatgaaattgcagaagaaaatatga

SEQ ID NO: 48 - protein, 522 aa, Chondromyces crocatus
MQLPSSTKILVVGGGPAGSTAATLLAREGFEVTLVEKAIFPRYHIGESLLISVQPIIDLLGAREAVEAHGFQRKKGVLWEWGGERWLFDWKKLRYDYTFHVKREEFDEILLRNAQKNGVKVFEGIDISRLEFDGERPVAAKWSKSSTGESGTIQFEFLLDASGRAGLMATQYLRSRMFMKAFQNVATWGYWKGATIPEVEVEGPITVGSIPYGWIWGIPLRDQTMSVGLVIHQELFKEKRATQSVEEIYHEGLKASPLFQDVVLKGATLEPQIRTETDYSYISRTLAGPGFFLVGDSGAFIDPLLSSGVHLAMHSALLAAASVKSIIAGEVDMASATEFYQRCYQGHFLRWALIVASFYEVNARKETYFWTAQQLAHEELGVFNMSQADMKDVFATMVSGVVDLGDAQNAGRLQKGAERVHQYLDDDGREEDVTALLQKSKQRIFEYLDRVKNRDSRAAMQRYKAGGTETFSMGLDADGAVGGLYVTTEPRLGLLRKVVEERAEAATEAPAPAAPPPAVAEV

SEQ ID NO: 49 - DNA, 1569 bp, Chondromyces crocatus
atgcaattgccatcttctactaagatcttggttgttggtggtggtccagctggttctactgctgctactttgttggctagagaaggtttcgaagttactttggttgaaaaggctatcttcccaagataccacatcggtgaatctttgttgatctctgttcaaccaatcatcgacttgttgggtgctagagaagctgttgaagctcacggtttccaaagaaagaagggtgttttgtgggaatggggtggtgaaagatggttgttcgactggaagaagttgagatacgactacactttccacgttaagagagaagaattcgacgaaatcttgttgagaaacgctcaaaagaacggtgttaaggttttcgaaggtatcgacatctctagattggaattcgacggtgaaagaccagttgctgctaagtggtctaagtcttctactggtgaatctggtactatccaattcgaattcttgttggacgcttctggtagagctggtttgatggctactcaatacttgagatctagaatgttcatgaaggctttccaaaacgttgctacttggggttactggaagggtgctactatcccagaagttgaagttgaaggtccaatcactgttggttctatcccatacggttggatctggggtatcccattgagagaccaaactatgtctgttggtttggttatccaccaagaattgttcaaggaaaagagagctactcaatctgttgaagaaatctaccacgaaggtttgaaggcttctccattgttccaagacgttgttttgaagggtgctactttggaaccacaaatcagaactgaaactgactactcttacatctctagaactttggctggtccaggtttcttcttggttggtgactctggtgctttcatcgacccattgttgtcttctggtgttcacttggctatgcactctgctttgttggctgctgcttctgttaagtctatcatcgctggtgaagttgacatggcttctgctactgaattctaccaaagatgttaccaaggtcacttcttgagatgggctttgatcgttgcttctttctacgaagttaacgctagaaaggaaacttacttctggactgctcaacaattggctcacgaagaattgggtgttttcaacatgtctcaagctgacatgaaggacgttttcgctactatggtttctggtgttgttgacttgggtgacgctcaaaacgctggtagattgcaaaagggtgctgaaagagttcaccaatacttggacgacgacggtagagaagaagacgttactgctttgttgcaaaagtctaagcaaagaatcttcgaatacttggacagagttaagaacagagactctagagctgctatgcaaagatacaaggctggtggtactgaaactttctctatgggtttggacgctgacggtgctgttggtggtttgtacgttactactgaaccaagattgggtttgttgagaaaggttgttgaagaaagagctgaagctgctactgaagctccagctccagctgctccaccaccagctgttgctgaagtttaa

SEQ ID NO: 50 - protein, 538 aa, Pseudomonas fluorescens
MNKPIKNIVIVGGGTAGWMAASYLVRALQQQANITLIESAAIPRIGVGEATIPSLQKVFFDFLGIPEREWMPQVNGAFKAAIKFVNWRKSPDPSRDDHFYHLFGNVPNCDGVPLTHYWLRKREQGFQQPMEYACYPQPGALDGKLAPCLSDGTRQMSHAWHFDAHLVADFLKRWAVERGVNRVVDEVVDVRLNNRGYISNLLTKEGRTLEADLFIDCSGMRGLLINQALKEPFIDMSDYLLCDSAVASAVPNDDARDGVEPYTSSIAMNSGWTWKIPMLGRFGSGYVFSSHFTSRDQATADFLKLWGLSDNQPLNQIKFRVGRNKRAWVNNCVSIGLSSCFLEPLESTGIYFIYAALYQLVKHFPDTSFDPRLSDAFNAEIVHMFDDCRDFVQAHYFTTSRDDTPFWLANRHDLRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRDGDAGLSRGQRGPKLAAQESL

SEQ ID NO: 51 - protein, 531 aa, Streptomyces albogriseolus
MDNRIKTVVILGGGTAGWMTAAYLGKALQNTVKIVVLEAPTIPRIGVGEATVPNLQRAFFDYLGIPEEEWMRECNASYKMAVKFINWRTPGEGSPDPRTLDDGHTDTFHHPFGLLPSADQIPLSHYWAAKRLQGETDENFDEACFADTAIMNAKKAPRFLDMRRATNYAWHFDASKVAAFLRNFAVTKQAVEHVEDEMTEVLTDERGFITALRTKSGRILQGDLFVDCSGFRGLLINKAMEEPFIDMSDHLLCNSAVATAVPHDDEKNGVEPYTSSIAMEAGWTWKIPMLGRFGSGHVYSDHFATQDEATLAFSKLWGLDPDNTEFNHVRFRVGRNRRAWVRNCVSVGLASCFVEPLESSGIYFIYAAIHMLAKHFPDKTFDKVLVDRFNREIEEMFDDTRDFLQAHYYFSPRVDTPFWRANKELKLADSIKDKVETYRAGLPVNLPVTDEGTYYGNFEAEFRNFWTNGSYYCIFAGLGLMPRNPLPALAYKPQSIAEAELLFADVKRKGDTLVESLPSTYDLLRQLHGAS

SEQ ID NO: 52 - protein, 603 aa, Dictyostelium discoideum
MDTNIINHYDIIIIGGGIAGLSATRHLLLKMPELSGRIAVIEPRSERRDNLDEDYKVGESTVEVSAMFFAKELELQDYLIENHPPKFSLQFHWPRELSKTDTIEDYYSTWAVKNPDIQAFQLNRCKIERDLLKMVIAQGAVYYHGRVRNVDNLDNDDMKSIDVEILSEVESGADFKLQQSIERITLTTDYIVDASGRNFTVGSRTDNILKDPKHLFGLDNASTWVRVKNTERSLFDFKSQDVTCSWTYDTNHFFGPGYWIWMIPLERGSRDYSIGVSYHRDKIQPSQLNSLDKFMSFLEKNQKLLYNLIKSGEIVDFHRWPKLAHTSKTFFSKNNWCVIGDAAAIFDPFYSTGMVMIAMEIECLTEMLKFKLSNAANDYHRRVEAFDKLIRCVTQINNHLIKDHSNHLGNASIMSWRIYFESSTYFSILLPAYIGKYHLCPIFSDHFTTDHENGLALRNQLLATLDYANENSINIGFMDNHRGGQLLGDWSPTSSWDYDHALSLAKYGHKRLNLPKCLSWSNFYLSLIICKLYYRVYGIGALWNSSFFKSLSNTTYRFSKFYFLSKLHSFNMIGTPNNDYYDKIQKDFKSYNYNQNNIVDWKY

SEQ ID NO: 53 - protein, 533 aa, Kutzneria sp. 744
MDDNRIRSILVLGGGTAGWMSACYLSKALGPGVEVTVLEAPSISRIRVGEATIPNLHKVFFDFLGIAEDEWMRECNASYKAAVRFVNWRTPGDGQATPRRRPDGRPDHFDHLFGQLPEHENLPLSQYWAHRRLNGLTDEPFDRSCYVQPELLDRKLSPRLMDGTKLASYAWHFDADLVADFLCRFAVQKLNVTHVQDVFTHADLDQRGHITAVNTESGRTLAADLFIDCSGFRSVLMGKVMQEPFLDMSKHLLNDRAVALMLPHDDEKVGIEPYTSSLAMRSGWSWKIPLLGRFGSGYVYSSQFTSQDEAAEELCRMWDVDPAEQTFNNVRFRVGRSRRAWVRNCVAIGVSAMFVEPLESTGLYFSYASLYQLVKHFPDKRFRPILADRFNREVATMYDDTRDFLQAHFSLSPRDDSEFWRACKELPFADGFAEKVEMYRAGLPVELPVTIDDGHYYGNFEAEFRNFWTNSNYYCIFAGLGFLPEHPLPVLEFRPEAVDRAEPVFAAVRRRTEELVATAPTMQAYLRRLHQGT

SEQ ID NO: 54 - protein, 505 aa, Kutzneria sp. 744
MTAAYLKTAFGDRLSITVVESSRIGTIGVGEATFSDIQHFFQFLNLREQDWMPACNATYKLGIRFENWRHVGHHFYQPFEQIRPVYGFPLTDWWLHDAPTDRFDTDCFVMPNLCEAGRSPRHLDGTLADEDFVEEGDELANRTMSEHQGKSQFPYAYHFEAALLAKFLTGYAVDRGVEHVVDDVLDVRLDQRGWIEHVVTAEHGEIHGDLFVDCTGFRGLLLNKALGVPFVSYQDTLPNDSAVALQVPLDMQRRGIVPNTTATAREAGWIWTIPLFGRVGTGYVYAKDYLSPEEAERTLREFVGPAAADVEANHIRMRIGRSQESWRNNCVAIGLSSGFVEPLESTGIFFIHHAIEQLVKHFPAADWNPKSRDMYNSAVAHVMDGIREFLVIHYRGAARADNQYWRDTKTRPLPDGLAERIECWQTQLPDTETIYPYYHGLPPYSYMCILMGGGAIRTPASAALALTDQGAAQKEFAAVRDRAAQLRDTLPSHYEYLARMRGLDV

SEQ ID NO: 55 - DNA, 1617 bp, Pseudomonas fluorescens
atgaacaagccaatcaagaacatcgttatcgttggtggtggtactgctggttggatggctgcttcttacttggttagagctttgcaacaacaagctaacatcactttgatcgaatctgctgctatcccaagaatcggtgttggtgaagctactatcccatctttgcaaaaggttttcttcgacttcttgggtatcccagaaagagaatggatgccacaagttaacggtgctttcaaggctgctatcaagttcgttaactggagaaagtctccagacccatctagagacgaccacttctaccacttgttcggtaacgttccaaactgtgacggtgttccattgactcactactggttgagaaagagagaacaaggtttccaacaaccaatggaatacgcttgttacccacaaccaggtgctttggacggtaagttggctccatgtttgtctgacggtactagacaaatgtctcacgcttggcacttcgacgctcacttggttgctgacttcttgaagagatgggctgttgaaagaggtgttaacagagttgttgacgaagttgttgacgttagattgaacaacagaggttacatctctaacttgttgactaaggaaggtagaactttggaagctgacttgttcatcgactgttctggtatgagaggtttgttgatcaaccaagctttgaaggaaccattcatcgacatgtctgactacttgttgtgtgactctgctgttgcttctgctgttccaaacgacgacgctagagacggtgttgaaccatacacttcttctatcgctatgaactctggttggacttggaagatcccaatgttgggtagattcggttctggttacgttttctcttctcacttcacttctagagaccaagctactgctgacttcttgaagttgtggggtttgtctgacaaccaaccattgaaccaaatcaagttcagagttggtagaaacaagagagcttgggttaacaactgtgtttctatcggtttgtcttcttgtttcttggaaccattggaatctactggtatctacttcatctacgctgctttgtaccaattggttaagcacttcccagacacttctttcgacccaagattgtctgacgctttcaacgctgaaatcgttcacatgttcgacgactgtagagacttcgttcaagctcactacttcactacttctagagacgacactccattctggttggctaacagacacgacttgagattgtctgacgctatcaaggaaaaggttcaaagatacaaggctggtttgccattgactactacttctttcgacgactctacttactacgaaactttcgactacgaattcaagaacttctggttgaacggtaactactactgtatcttcgctggtttgggtatgttgccagacagatctttgccattgttgcaacacagaccagaatctatcgaaaaggctgaagctatgttcgcttctatcagaagagaagctgaaagattgagaacttctttgccaactaactacgactacttgagatctttgagagacggtgacgctggtttgtctagaggtcaaagaggtccaaagttggctgctcaagaatctttgtaa

SEQ ID NO: 56 - DNA, 1596 bp, Streptomyces albogriseolus
atggacaacagaatcaagactgttgttatcttgggtggtggtactgctggttggatgactgctgcttacttgggtaaggctttgcaaaacactgttaagatcgttgttttggaagctccaactatcccaagaatcggtgttggtgaagctactgttccaaacttgcaaagagctttcttcgactacttgggtatcccagaagaagaatggatgagagaatgtaacgcttcttacaagatggctgttaagttcatcaactggagaactccaggtgaaggttctccagacccaagaactttggacgacggtcacactgacactttccaccacccattcggtttgttgccatctgctgaccaaatcccattgtctcactactgggctgctaagagattgcaaggtgaaactgacgaaaacttcgacgaagcttgtttcgctgacactgctatcatgaacgctaagaaggctccaagattcttggacatgagaagagctactaactacgcttggcacttcgacgcttctaaggttgctgctttcttgagaaacttcgctgttactaagcaagctgttgaacacgttgaagacgaaatgactgaagttttgactgacgaaagaggtttcatcactgctttgagaactaagtctggtagaatcttgcaaggtgacttgttcgttgactgttctggtttcagaggtttgttgatcaacaaggctatggaagaaccattcatcgacatgtctgaccacttgttgtgtaactctgctgttgctactgctgttccacacgacgacgaaaagaacggtgttgaaccatacacttcttctatcgctatggaagctggttggacttggaagatcccaatgttgggtagattcggttctggtcacgtttactctgaccacttcgctactcaagacgaagctactttggctttctctaagttgtggggtttggacccagacaacactgaattcaaccacgttagattcagagttggtagaaacagaagagcttgggttagaaactgtgtttctgttggtttggcttcttgtttcgttgaaccattggaatcttctggtatctacttcatctacgctgctatccacatgttggctaagcacttcccagacaagactttcgacaaggttttggttgacagattcaacagagaaatcgaagaaatgttcgacgacactagagacttcttgcaagctcactactacttctctccaagagttgacactccattctggagagctaacaaggaattgaagttggctgactctatcaaggacaaggttgaaacttacagagctggtttgccagttaacttgccagttactgacgaaggtacttactacggtaacttcgaagctgaattcagaaacttctggactaacggttcttactactgtatcttcgctggtttgggtttgatgccaagaaacccattgccagctttggcttacaagccacaatctatcgctgaagctgaattgttgttcgctgacgttaagagaaagggtgacactttggttgaatctttgccatctacttacgacttgttgagacaattgcacggtgcttcttaa

SEQ ID NO: 57 - DNA, 1812 bp, Dictyostelium discoideum
atggacactaacatcatcaaccactacgacatcatcatcatcggtggtggtatcgctggtttgtctgctactagacacttgttgttgaagatgccagaattgtctggtagaatcgctgttatcgaaccaagatctgaaagaagagacaacttggacgaagactacaaggttggtgaatctactgttgaagtttctgctatgttcttcgctaaggaattggaattgcaagactacttgatcgaaaaccacccaccaaagttctctttgcaattccactggccaagagaattgtctaagactgacactatcgaagactactactctacttgggctgttaagaacccagacatccaagctttccaattgaacagatgtaagatcgaaagagacttgttgaagatggttatcgctcaaggtgctgtttactaccacggtagagttagaaacgttgacaacttggacaacgacgacatgaagtctatcgacgttgaaatcttgtctgaagttgaatctggtgctgacttcaagttgcaacaatctatcgaaagaatcactttgactactgactacatcgttgacgcttctggtagaaacttcactgttggttctagaactgacaacatcttgaaggacccaaagcacttgttcggtttggacaacgcttctacttgggttagagttaagaacactgaaagatctttgttcgacttcaagtctcaagacgttacttgttcttggacttacgacactaaccacttcttcggtccaggttactggatctggatgatcccattggaaagaggttctagagactactctatcggtgtttcttaccacagagacaagatccaaccatctcaattgaactctttggacaagttcatgtctttcttggaaaagaaccaaaagttgttgtacaacttgatcaagtctggtgaaatcgttgacttccacagatggccaaagttggctcacacttctaagactttcttctctaagaacaactggtgtgttatcggtgacgctgctgctatcttcgacccattctactctactggtatggttatgatcgctatggaaatcgaatgtttgactgaaatgttgaagttcaagttgtctaacgctgctaacgactaccacagaagagttgaagctttcgacaagttgatcagatgtgttactcaaatcaacaaccacttgatcaaggaccactctaaccacttgggtaacgcttctatcatgtcttggagaatctacttcgaatcttctacttacttctctatcttgttgccagcttacatcggtaagtaccacttgtgtccaatcttctctgaccacttcactactgaccacgaaaacggtttggctttgagaaaccaattgttggctactttggactacgctaacgaaaactctatcaacatcggtttcatggacaaccacagaggtggtcaattgttgggtgactggtctccaacttcttcttgggactacgaccacgctttgtctttggctaagtacggtcacaagagattgaacttgccaaagtgtttgtcttggtctaacttctacttgtctttgatcatctgtaagttgtactacagagtttacggtatcggtgctttgtggaactcttctttcttcaagtctttgtctaacactacttacagattctctaagttctacttcttgtctaagttgcactctttcaacatgatcggtactccaaacaacgactactacgacaagatccaaaaggacttcaagtcttacaactacaaccaaaacaacatcgttgactggaagtactaa

SEQ ID NO: 58 - DNA, 1602 bp, Kutzneria sp. 744
atggacgacaacagaatcagatctatcttggttttgggtggtggtactgctggttggatgtctgcttgttacttgtctaaggctttgggtccaggtgttgaagttactgttttggaagctccatctatctctagaatcagagttggtgaagctactatcccaaacttgcacaaggttttcttcgacttcttgggtatcgctgaagacgaatggatgagagaatgtaacgcttcttacaaggctgctgttagattcgttaactggagaactccaggtgacggtcaagctactccaagaagaagaccagacggtagaccagaccacttcgaccacttgttcggtcaattgccagaacacgaaaacttgccattgtctcaatactgggctcacagaagattgaacggtttgactgacgaaccattcgacagatcttgttacgttcaaccagaattgttggacagaaagttgtctccaagattgatggacggtactaagttggcttcttacgcttggcacttcgacgctgacttggttgctgacttcttgtgtagattcgctgttcaaaagttgaacgttactcacgttcaagacgttttcactcacgctgacttggaccaaagaggtcacatcactgctgttaacactgaatctggtagaactttggctgctgacttgttcatcgactgttctggtttcagatctgttttgatgggtaaggttatgcaagaaccattcttggacatgtctaagcacttgttgaacgacagagctgttgctttgatgttgccacacgacgacgaaaaggttggtatcgaaccatacacttcttctttggctatgagatctggttggtcttggaagatcccattgttgggtagattcggttctggttacgtttactcttctcaattcacttctcaagacgaagctgctgaagaattgtgtagaatgtgggacgttgacccagctgaacaaactttcaacaacgttagattcagagttggtagatctagaagagcttgggttagaaactgtgttgctatcggtgtttctgctatgttcgttgaaccattggaatctactggtttgtacttctcttacgcttctttgtaccaattggttaagcacttcccagacaagagattcagaccaatcttggctgacagattcaacagagaagttgctactatgtacgacgacactagagacttcttgcaagctcacttctctttgtctccaagagacgactctgaattctggagagcttgtaaggaattgccattcgctgacggtttcgctgaaaaggttgaaatgtacagagctggtttgccagttgaattgccagttactatcgacgacggtcactactacggtaacttcgaagctgaattcagaaacttctggactaactctaactactactgtatcttcgctggtttgggtttcttgccagaacacccattgccagttttggaattcagaccagaagctgttgacagagctgaaccagttttcgctgctgttagaagaagaactgaagaattggttgctactgctccaactatgcaagcttacttgagaagattgcaccaaggtacttaa

SEQ ID NO: 59 - DNA, 1518 bp, Kutzneria sp. 744
atgactgctgcttacttgaagactgctttcggtgacagattgtctatcactgttgttgaatcttctagaatcggtactatcggtgttggtgaagctactttctctgacatccaacacttcttccaattcttgaacttgagagaacaagactggatgccagcttgtaacgctacttacaagttgggtatcagattcgaaaactggagacacgttggtcaccacttctaccaaccattcgaacaaatcagaccagtttacggtttcccattgactgactggtggttgcacgacgctccaactgacagattcgacactgactgtttcgttatgccaaacttgtgtgaagctggtagatctccaagacacttggacggtactttggctgacgaagacttcgttgaagaaggtgacgaattggctaacagaactatgtctgaacaccaaggtaagtctcaattcccatacgcttaccacttcgaagctgctttgttggctaagttcttgactggttacgctgttgacagaggtgttgaacacgttgttgacgacgttttggacgttagattggaccaaagaggttggatcgaacacgttgttactgctgaacacggtgaaatccacggtgacttgttcgttgactgtactggtttcagaggtttgttgttgaacaaggctttgggtgttccattcgtttcttaccaagacactttgccaaacgactctgctgttgctttgcaagttccattggacatgcaaagaagaggtatcgttccaaacactactgctactgctagagaagctggttggatctggactatcccattgttcggtagagttggtactggttacgtttacgctaaggactacttgtctccagaagaagctgaaagaactttgagagaattcgttggtccagctgctgctgacgttgaagctaaccacatcagaatgagaatcggtagatctcaagaatcttggagaaacaactgtgttgctatcggtttgtcttctggtttcgttgaaccattggaatctactggtatcttcttcatccaccacgctatcgaacaattggttaagcacttcccagctgctgactggaacccaaagtctagagacatgtacaactctgctgttgctcacgttatggacggtatcagagaattcttggttatccactacagaggtgctgctagagctgacaaccaatactggagagacactaagactagaccattgccagacggtttggctgaaagaatcgaatgttggcaaactcaattgccagacactgaaactatctacccatactaccacggtttgccaccatactcttacatgtgtatcttgatgggtggtggtgctatcagaactccagcttctgctgctttggctttgactgaccaaggtgctgctcaaaaggaattcgctgctgttagagacagagctgctcaattgagagacactttgccatctcactacgaatacttggctagaatgagaggtttggacgtttaa

(19) United States
(12) Patent Application Publication
(10) Pub. No.: US 2022/0403428 A1
(43) Pub. Date: Dec. 22, 2022

(54) YEAST CELLS AND METHODS FOR PRODUCTION OF TRYPTOPHAN DERIVATIVES

(71) Applicant: Danmarks Tekniske Universitet, Kgs. Lyngby (DK)

(72) Inventors: Irina Borodina, Nivå (DK); Nicholas Milne, Frederiksberg C (DK); Steven van der Hoek, Kgs. Lyngby (DK); Morten Otto Alexander Sommer, Hellerup (DK); Jérémy Christophe Daniel Armetta, Copenhagen NV (DK); Philip Tinggaard Thomsen, Kgs. Lyngby (DK)

(21) Appl. No.: 17/642,815

(22) PCT Filed: Sep. 16, 2020
(86) PCT No.: PCT/EP2020/075823

§ 371 Date: Mar. 14, 2022

(30) Foreign Application Priority Data
Sep. 16, 2019 (EP) 19197432.8
Mar. 20, 2020 (EP) 20164388.9

(51) Int. Cl. C12P 13/22; C12N 15/81

(52) U.S. Cl. CPC C12P 13/227; C12N 15/81; C12Y 105/0103

(57) ABSTRACT

The present disclosure relates to methods for production of 4-hydroxytryptamine and derivatives thereof in a yeast cell. Herein are also disclosed methods for production of halogenated tryptophans and derivatives thereof in a cell. Herein are also disclosed methods for production of methylated tryptamine. The disclosure also provides nucleic acid constructs and cells useful for performing the present methods.

### TECHNICAL FIELD

Herein are disclosed methods for production of 4-hydroxytryptamine and derivatives thereof in a yeast cell. Herein are also disclosed methods for production of halogenated tryptophans and derivatives thereof in a cell. Herein are also disclosed methods for production of methylated tryptamine. The disclosure also provides nucleic acid constructs and cells useful for performing the present methods.

### BACKGROUND

Psilocybin is a tryptamine-derived psychoactive alkaloid found in the fungal genus Psilocybe, among others, and is the active ingredient in so-called ‘magic mushrooms’. Psilocybin itself is not psychoactive—rather it is the dephosphorylated derivative psilocin that causes the hallucinogenic effect. Psilocybin is rapidly dephosphorylated to psilocin following ingestion in the mucosa by alkaline phosphatases and nonspecific esterases. Psilocin is structurally similar to human signaling molecules such as serotonin, and has been shown to bind to over 15 human serotonin-related receptors.

Clinical trials have recently recognized psilocybin as a promising candidate for the treatment of various psychological and neurological afflictions. Preliminary results suggest that psilocybin assisted treatment may be a good candidate for managing substance addiction (Bogenschutz et al., 2015; Riaz et al., 2016), anxiety in terminally ill patients (Grob et al., 2011), cluster headaches (Tyls̆ et al., 2014), and treatment-resistant depression (Carhart-Harris et al., 2018). Psilocybin seems to be a particularly interesting candidate for “treatment resistant depression”—a term applied to the 13% of patients with Major Depressive Disorder (MDD) who relapse, in spite of four rounds of traditional treatment (Rush et al., 2006). Approximately 16 million Americans carried the MDD diagnosis in 2016, indicating a large number of people with untreated mental illness (Tice, 2017).

Unfortunately, the content of psilocybin and psilocin in hallucinogenic mushrooms is too low (0.2%-1% dry weight) to make extraction a commercially viable option (Tyls̆ et al., 2014), and chemical synthesis is complicated and expensive (Nichols and Frescas, 1999). Although the chemical synthesis of psilocybin has been improved since its discovery by Hoffman et al. in 1959, who achieved final yields of 20% of semi pure psilocybin, it continues to challenge chemists primarily due to the difficulty of the last synthetic step; the phosphorylation of psilocin (Nichols and Frescas, 1999).

Therefore, psilocybin is currently obtained mainly through complex and expensive chemical synthesis. Accordingly, there is a demand for effective and low cost production of psilocybin for pharmaceutical applications.

Halogenated compounds constitute a large fraction of pharmaceuticals on the market and in development due to the ability of halogenation to modulate the properties of a lead drug candidate. Since many natural products with therapeutic properties are synthesized from tryptamine, for example the Catharanthus roseus anticancer agent vinblastine, halogenated tryptamine derivatives might be of great therapeutic interest.

Biotechnological production of halogenated tryptamines in a cell factory could serve both as a production method and as a means for drug discovery. Halogenated tryptophan, halogenated tryptamine, halogenated N-methylated tryptamine, halogenated N,N-dimethyltryptamine and halogenated N,N,N-trimethyltryptamine are expected to have enhanced therapeutic properties compared to their non-halogenated counterparts. Accordingly, a method for effective and low cost production of these compounds for pharmaceutical applications is desirable.

DMT is a psychoactive tryptamine derivative present in a wide range of plants as well as in mammals, where it is synthesized from tryptamine by the SAM-dependent indole N-methyltransferase INMT.

### SUMMARY

The invention is as defined in the claims.

Herein is provided a method for the production of 4-hydroxytryptamine and derivatives thereof, such as psilocybin, in a yeast cell by expression of a heterologous biosynthesis pathway sourced from Psilocybe cubensis. The inventors have achieved improved product titers by supplementing the pathway with a novel cytochrome P450 reductase from Psilocybe cubensis. Microbial based production of 4-hydroxytriptamine and derivatives thereof, including psilocybin, can be performed at reduced financial and environmental costs compared to methods known in the art.

In one aspect, the present invention provides a yeast cell capable of producing 4-hydroxytryptamine and optionally derivatives thereof, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,
   wherein the tryptamine 4-monooxygenase and the cytochrome P450 reductase together catalyse the conversion of tryptamine to 4-hydroxytryptamine,
   whereby the yeast cell is capable of converting tryptophan to 4-hydroxytryptamine.

In another aspect, the present invention provides a method of producing 4-hydroxytryptamine and optionally derivatives thereof in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

In one aspect, the present invention provides 4-hydroxytryptamine and derivatives thereof obtainable by the methods disclosed herein.

In one aspect, the present invention provides a nucleic acid construct for modifying a yeast cell, said construct comprising:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto; and
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

In another aspect, the present invention provides a kit of parts comprising:
the yeast cell as disclosed herein and instructions for use; and/or
the nucleic acid construct as disclosed herein and instructions for use; and optionally the yeast cell to be modified.

Herein are also disclosed methods for the production of halogenated tryptophans and derivatives thereof in a cell by expression of a heterologous biosynthesis pathway sourced from Chondromyces crocatus, Streptomyces rugosporus, Streptomyces toxytricini, Lechevalieria aerocolonigenes, Catharanthus roseus and Oryctolagus cuniculus. De novo cell-based production of halogenated tryptophan and derivatives thereof in cells, such as yeast cells, can be performed at low financial and environmental costs.

In one aspect, is provided a cell capable of producing a halogenated tryptophan, wherein the halogenated tryptophan is a tryptophan substituted with one, two or three halogen atoms, and optionally derivatives thereof, in the presence of a halogen or derivatives thereof, said cell expressing at least one of:
a tryptophan-2-halogenase (EC 1.14.14), preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto, and optionally expressing a flavin reductase (EC 1.5.1.30), preferably a heterologous flavin reductase, such as LaRebF (SEQ ID NO: 35) or a functional variant thereof having at least 80% homology, such as at least 85%, such as at least 90%, such as at least 95% homology thereto,
   whereby the cell is capable of converting tryptophan into a halogenated tryptophan and optionally derivatives thereof, a dihalogenated tryptophan and optionally derivatives thereof, or a trihalogenated tryptophan and optionally derivatives thereof, preferably wherein the cell is a microorganism or a plant cell.

In another aspect, is provided a method of producing a halogenated tryptophan wherein the halogenated tryptophan is a tryptophan substituted with one, two or three halogen atoms, and optionally derivatives thereof, in a cell, preferably wherein the cell is a microorganism or a plant cell, said method comprising the steps of providing a cell and incubating said cell in the presence of a halogen, wherein the cell expresses at least one of:
a tryptophan-2-halogenase (EC 1.14.14), preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto, and optionally a flavin reductase, preferably a heterologous flavin reductase (EC: EC 1.5.1.30), such as LaRebF (SEQ ID NO: 35), or a functional variant thereof having at least 80% homology, such as at least 85%, such as at least 90%, such as at least 95% homology thereto.

In one aspect, are provided halogenated tryptophans and derivatives thereof, in particular a halogenated tryptophan, a dihalogenated tryptophan, a trihalogenated tryptophan, a halogenated tryptamine, a dihalogenated tryptamine, a trihalogenated tryptamine, a halogenated N-methyltryptamine, a halogenated N,N-dimethyltryptamine, a halogenated N,N,N-trimethyltryptamine, a dihalogenated N-methyltryptamine, a dihalogenated N,N-dimethyltryptamine, a dihalogenated N,N,N-trimethyltryptamine, a trihalogenated N-methyltryptamine, a trihalogenated N,N-dimethyltryptamine and a trihalogenated N,N,N-trimethyltryptamine obtainable by the methods disclosed herein.

In one aspect, is provided a nucleic acid construct for modifying a cell, said construct comprising at least one of:
a polynucleotide encoding a tryptophan-2-halogenase (EC 1.14.14), preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a polynucleotide encoding a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a polynucleotide encoding a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto, or
a polynucleotide encoding a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto,
a polynucleotide encoding a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto, and optionally a polynucleotide encoding a flavin reductase (EC 1.5.1.30), preferably a heterologous flavin reductase, such as LaRebF (SEQ ID NO: 35) or a functional variant thereof having at least 80% homology, such as at least 85%, such as at least 90%, such as at least 95% homology thereto.

In another aspect, the present invention provides a kit of parts comprising:
the cell as disclosed herein and instructions for use; and/or
the nucleic acid construct as disclosed herein and instructions for use; and optionally the cell to be modified.

Herein is also provided a cell capable of producing N-methyltryptamine, N,N-dimethyltryptamine and/or N,N,N-trimethyltryptamine, preferably wherein the cell is a microorganism or a plant cell, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine; and
an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase is capable of converting tryptamine to N-methyltryptamine, to N,N-dimethyltryptamine, and/or to N,N,N-trimethyltryptamine,

whereby the cell is capable of producing N-methyltryptamine, N,N-dimethyltryptamine, and/or N,N,N-trimethyltryptamine.

Also provided herein is a method for producing N-methyltryptamine, N,N-dimethyltryptamine and/or N,N,N-trimethyltryptamine in a cell, preferably wherein the cell is a microorganism or a plant cell, said method comprising the steps of providing a cell and incubating said cell in a medium, wherein the cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine; and

an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase is capable of converting tryptamine to N-methyltryptamine, to N,N-dimethyltryptamine, and/or to N, N, N-trimethyltryptamine.

Also provided herein are nucleic acid constructs comprising:
a polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, such as a polynucleotide comprising or consisting of SEQ ID NO: 6 or a homologue thereof having at least 80% homology, such as at least 85%, such as at least 90%, such as at least 95% homology thereto
a polynucleotide encoding an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, such as a polynucleotide comprising or consisting of SEQ ID NO: 36 or a homologue thereof having at least 80% homology, such as at least 85%, such as at least 90%, such as at least 95% homology thereto.

### DESCRIPTION OF THE DRAWINGS

FIG. 1. Psilocybin route of administration in humans. After biosynthesis in Psilocybe mushroom species (or in the context of the present disclosure in a yeast such as Saccharomyces cerevisiae), psilocybin is typically administered by oral ingestion. Upon consumption, psilocybin acts as a prodrug where alkaline phosphatases or non-specific esterases convert the molecule to the bioactive psilocin. Psilocin then typically exerts its hallucinogenic or therapeutic effect by crossing the blood-brain barrier and interacting with serotonin receptors. Psilocin is eventually removed from the body via glucuronidation and excretion through the kidneys.

FIG. 2. Psilocybin biosynthesis in S. cerevisiae. An exemplary heterologous biosynthetic pathway for production of psilocybin begins with the native production of tryptophan which itself is derived from metabolites produced via glycolysis, the pentose phosphate pathway, and the shikimate pathway. Multiple arrows represent multiple enzymatic reactions grouped for simplicity. The figure shows specific enzymes obtained from Catharanthus roseus (CrTdc), Psilocybe cubensis (PcPsiK, PcPsiM, PcPsiH, PcCpr) and Bos taurus (BtAANAT). These are exemplary only and may be replaced by corresponding enzymes originating from different organisms.

FIG. 3. De novo psilocybin production in S. cerevisiae (A). LC-MS chromatograms confirming psilocybin, psilocin and tryptamine production in ST9327 compared to wild-type control strain ST9326 using authentic analytical standards. (B). Corresponding mass spectra for psilocybin, psilocin and tryptamine peaks in ST9327.

FIG. 4. Improved De novo psilocybin biosynthesis in S. cerevisiae. (A) and (C). Introduction of the heterologous biosynthesis pathway and corresponding final titers in micro-titer plate cultivation. ST9326; Wild-type parental strain, ST9327; basic heterologous pathway (CrTdc, PcPsiH, PcPsiK, PcPsiM), ST9649; basic heterologous pathway+overexpression of native S. cerevisiae NCP1 gene (pTEF1->NCP1), ST9330; basic heterologous pathway+A. thaliana CPR (AtAtr2), ST9329; basic heterologous pathway+P. cubensis CPR expressed from TEF2 promoter (pTEF2->PcCpr), ST9328; basic heterologous pathway+P. cubensis CPR expressed from TEF1 promoter (pTEF1->PcCpr). Y-axis shows the titer in mg/L. (B) and (D). Iterative strain improvement to increase tryptophan availability and corresponding final titers in micro-titer plate cultivation. Gene names represent genes that were expressed from strong constitutive promoters. Strains were cultivated in synthetic FIT media for 72 h and subjected to acetonitrile extraction and analysis by LC-MS. Media was supplemented with uracil when required. Data is presented as averages and standard deviations from biological duplicates. *; Not detected. Heterologous pathway: strain expressing Crtdc, PcpsiH, PcpsiK, PcpsiM and Pccpr from the TEF1 promoter. Y-axis shows the titer in mg/L.

FIG. 5. Production of 4-hydroxytryptamine derivatives in engineered S. cerevisiae strains. LC-MS chromatograms and corresponding mass spectra for (A) Norbaeocystin, (B) Baeocystin, (C) Norpsilocin, (D) Dephosphorylated aeruginascin, and (E)N-acetyl-4-hydroxytryptamine produced in engineered S. cerevisiae strains ST9326 (Wild-type control), ST9328 (Crtdc, PcpsiH, Pccpr, PcpsiK, PcpsiM), ST9335 (Crtdc, PcpsiH, Pccpr, PcpsiK, PcpsiM multi-copy), ST9442 (Crtdc, PcpsiH, Pccpr, BtAANAT multi-copy).

FIG. 6. Expression of a P. cubensis cytochrome b5 (PcCYB5) increases production of psilocybin and pathway intermediates in S. cerevisiae. Strains ST9328 (Psilocybin biosynthetic pathway+PcCPR) and ST9740 (Psilocybin biosynthetic pathway+PcCPR+PcCYB5) were cultivated in synthetic media (Delft) with addition of 5 g/L tryptophan for 3 days at 30° C. and 200 RPM. Samples analyzed by supernatant extraction and analysis by LC-MS. Psilocybin and tryptamine titers were determined by comparison with authentic analytical standards, while for 4-hydroxytryptamine, norbaeocystin and baeocystin peak areas matching the expected m/z and fragmentation pattern of the metabolite of interest are shown.

FIG. 7. Effects of knock-out of SPE2 and ERGO in psilocybin producing strain ST9316. ST9316 expresses the full psilocybin biosynthesis pathway, P. cubensis cytochrome P450 reductase (PcCPR). Furthermore, it has been engineered to increase psilocybin production through knock-out of RIC1 and overexpression of ARO1, ARO2. Cultivations of engineered strains were carried out in synthetic minimal media and metabolites were extracted using the extracellular protocol with water as the solvent and analysed by LC-MS with analytical standards. All values are means of biological duplicates and error bars represent the calculated standard deviations.

FIG. 8. Effects of overexpression of native S. cerevisiae POS5 gene on metabolite production in psilocybin producing strain ST9328. ST9328 expresses the full psilocybin biosynthetic pathway and cytochrome p450 reductase from P. cubensis. The recombinant strain was inoculated from precultures into synthetic minimal media and after three days of cultivation, metabolites were extracted and analysed by LC-MS. Metabolite levels are reported as concentrations for metabolites analyzed with analytical standards. For metabolites analysed without analytical standards, levels are reported as normalized areas, meaning that peak areas have been normalized with respect to the highest measured peak area of each respective metabolite. All values are mean values of biological duplicates and error bars represent the calculated standard deviations.

FIG. 9. Glutamine supplementation improves psilocybin production. Cultivation of ST9328 (Psilocybin biosynthetic pathway+pTEF1->PcCPR) in synthetic media (delft) with addition of 5 g/L ammonium sulfate 20 g/L glucose and 5 g/L glutamine as indicated. Strain was cultivated for 3 days at 30° C. and 200 RPM. Samples were analyzed by acetonitrile extraction and analysis by LC-MS. Psilocybin and psilocin titers were determined by comparison with authentic analytical standards.

FIG. 10. De novo halogenation of tryptophan in S. cerevisiae. Expression of heterologous halogenases leads to bromination and chlorination at positions 5, 6, and 7. Expression of heterologous flavin reductase increases production by 100 fold. Expression of CrTdc leads to conversion of halogenated tryptophan to halogenated tryptamine.

FIG. 11. Metabolite production in recombinant yeast strains expressing tryptophan-5-halogenase (SrPyrH) and/or tryptophan-6-halogenase (SttH). The halogenases were expressed in absence and presence of the partner flavin reductase LaRebF and tryptamine decarboxylase CrTdc as indicated. “+” denotes that a single copy of the relevant gene was integrated into the genome. Strains were cultivated in synthetic minimal media supplemented with 25 mM KCl or 25 mM KBr. Metabolites from strains without a CrTdc gene overexpression were extracted using the intracellular extraction protocol. Metabolites for strains overexpressing CrTdc were extracted using the extracellular extraction protocol and metabolites from all strains were analyzed by LC-MS. For metabolites analyzed without an analytical standard, levels are reported as normalized peak areas, meaning that areas have been normalized with respect to the highest measured peak area of the metabolite. Peak identity was determined by matching the m/z and fragmentation pattern with the metabolite of interest. NA*: normalized area. Trp: tryptophan; Cl-Trp: chlorotryptophan; Br-Trp: bromotryptophan; Di-Cl-Trp: dichlorotryptophan; Di-Br-Trp: dibromotryptophan; Cl-Tryptamine: chlorotryptamine; Br-Tryptamine: bromotryptamine.

FIG. 12. Metabolite production in recombinant yeast strains ST9337 expressing CrTDC and ST9647 expressing CrTDC and OcINMT. Strains were cultivated in synthetic minimal media and metabolites were extracted using the extracellular extraction protocol and analyzed by LC-MS. Levels of N-methyltryptamine (NMT), N,N-dimethyltryptamine (DMT) and N,N,N-trimethyltryptamine are reported as LC-MS peak areas.

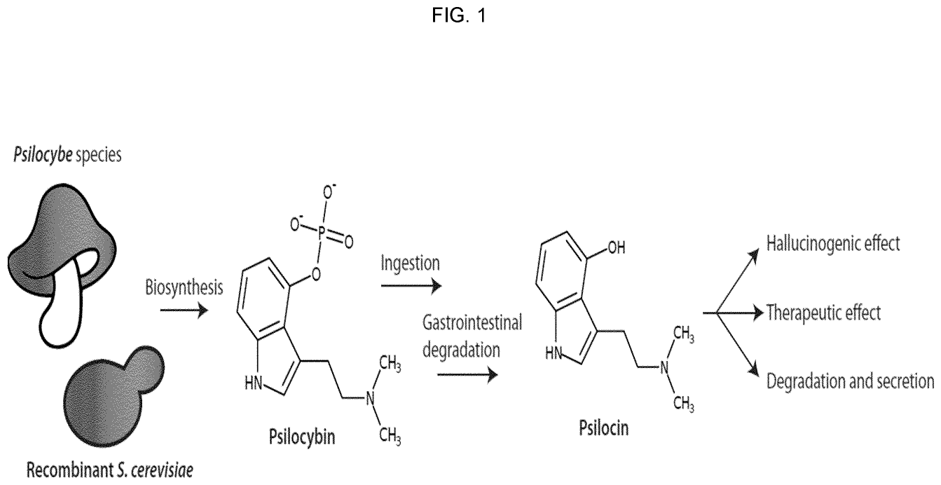

### DETAILED DESCRIPTION

Herein are disclosed yeast cells useful for production of 4-hydroxytryptamine and derivatives thereof, as well as methods, nucleic acid constructs and kits for production of 4-hydroxytryptamine and derivatives thereof.

Herein is provided a yeast cell capable of producing 4-hydroxytryptamine and optionally derivatives thereof, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,
   wherein the tryptamine 4-monooxygenase and the cytochrome P450 reductase together catalyse the conversion of tryptamine to 4-hydroxytryptamine,
      whereby the yeast cell is capable of converting tryptophan to 4-hydroxytryptamine.

Herein is also provided a method of producing 4-hydroxytryptamine and optionally derivatives thereof in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

Herein is also provided 4-hydroxytryptamine, norbaeocystin, baeocystin, norpsilocin, psilocybin, psilocin, aeruginascin, dephosphorylated aeruginascin or N-acetyl-4-hydroxytryptamine obtainable by the methods disclosed herein.

Herein is also provided a nucleic acid construct for modifying a yeast cell, said construct comprising:
a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59) (SEQ ID NO: 7), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto; and
a third polynucleotide encoding a cytochrome P450 reductase (EC 1.6.2.4) (SEQ ID NO:8), preferably a heterologous cytochrome P450 reductase such as PcCpr (SEQ ID NO: 3) or a functional variant thereof having at least 80% homology, such as at least 85%, such as at least 90%, such as at least 95% homology thereto.

Herein is also provided a kit of parts comprising:
the yeast cell as disclosed herein and instructions for use; and/or
the nucleic acid construct as disclosed herein and instructions for use; and optionally the yeast cell to be modified.

### Definitions

Tdc: tryptophan decarboxylase (EC 4.1.1.105)

L-tryptophan+H+→CO₂+tryptamine

The tryptophan decarboxylase (Tdc) converts L-tryptophan into tryptamine.

The enzyme Tdc preferably originates from the organism Catharanthus roseus.

PsiH: Tryptamine 4-monooxygenase (EC 1.14.99.59)

tryptamine+reduced acceptor+O₂=4-hydroxytryptamine+acceptor+H₂O

Tryptamine 4-monooxygenase (PsiH) converts tryptamine into 4-hydroxytryptamine.

The enzyme PsiH preferably originates from the organism Psilocybe cubensis.

Cpr: cytochrome P450 reductase (EC 1.6.2.4) catalyses the reaction NADPH+H⁺+n oxidized hemoprotein=NADP⁺+n reduced hemoprotein Cytochrome P450 reductase (Cpr) converts oxidized hemoprotein into reduced hemoprotein.

The enzyme Cpr preferably originates from the organism Psilocybe cubensis.

PsiK: 4-hydroxy tryptamine kinase (EC 2.7.1.222)

ATP+4-hydroxytryptamine→ADP+norbaeocystin+H⁺

4-hydroxy tryptamine kinase (PsiK) converts 4-hydroxytryptamine into norbaeocystin.

The enzyme PsiK preferably originates from the organism Psilocybe cubensis.

AANAT: serotonin N-acetyltransferase (EC 2.3.1.87)

Acetyl-CoA+a 2-arylethylamine<=>CoA+an N-acetyl-2-arylethylamine

The enzyme catalyses conversion of a 2-arylethylamine to the corresponding N-acetyl-2-arylethylamine in the presence of acetyl-CoA. For example it catalyses the conversion of 4-hydroxytryptamine into N-acetyl-4-hydroxytryptamine.

The enzyme AANAT preferably originates from the organism Bos taurus.

PsiM: Psilocybin synthase (EC 2.1.1.345) 2 S-adenosyl-L-methionine+norbaeocystin→2 S-adenosyl-L-homocysteine+psilocybin+2 H⁺

PsiM enzyme catalyses 2 (or 3) consecutive N-methylation steps; Norbaeocystin->Baeocystin->Psilocybin->Aeruginascin

The enzyme PsiM preferably originates from the organism Psilocybe cubensis.

4-hydroxytryptamine derivatives: the term herein refers to compounds obtainable from 4-hydroxytryptamine, such as psilocybin, psilocin, norbaeocystin, N-acetyl-4-hydroxytryptamine, baeocystin, norpsilocin, aeruginascin and dephosphorylated aeruginascin.

Functional variant: the term is herein applied to functional variants of enzymes, i.e. modified versions of the enzyme which retain some or all the catalytic activity of the enzyme. Functional variants may have been modified by introducing mutations which confer e.g. increased activity, a change in intracellular localisation, prolonged half-life, among others, but retain the ability to perform the same enzymatic reaction as the enzymes they are derived from. The mutations resulting in modified activity may be in the genes coding for the enzymes, including in their promoter region.

CmdE: tryptophan-2-halogenase (EC 1.14.14)

tryptophan+FADH₂+X⁻+O₂+H⁺2-X-L-tryptophan+FAD+2 H₂O,

where X is any halogen selected from the group consisting of fluorine, bromine, iodine and chlorine.

The tryptophan-2-halogenase replaces the hydrogen at position 2 of the indole ring of tryptophan with a halogen atom.

The enzyme CmdE preferably originates from the organism Chondromyces crocatus.

PyrH: tryptophan-5-halogenase (EC 1.14.19.58)

tryptophan+FADH₂+X⁻+O₂+H⁺5-X-L-tryptophan+FAD+2 H₂O,

where X is any halogen selected from the group consisting of fluorine, bromine, iodine and chlorine.

The tryptophan-5-halogenase replaces the hydrogen at position 5 of the indole ring of tryptophan with a halogen atom.

The enzyme PyrH preferably originates from the organism Streptomyces rugosporus.

tH: tryptophan-6-halogenase (EC 1.14.19.59)

tryptophan+FADH₂+X⁻+O₂+H⁺6-X-L-tryptophan+FAD+2 H₂O,

where X is any halogen selected from the group consisting of fluorine, bromine, iodine and chlorine.

The tryptophan-6-halogenase replaces the hydrogen at position 6 of the indole ring of tryptophan with a halogen atom.

The enzyme tH preferably originates from the organism Streptomyces toxytricini.

RebH: tryptophan-7-halogenase (EC 1.14.19.9)

tryptophan+FADH₂+X⁻+O₂+H⁺7-X-L-tryptophan+FAD+2 H₂O,

where X is any halogen selected from the group consisting of fluorine, bromine, iodine and chlorine.

The tryptophan-7-halogenase replaces the hydrogen at position 7 of the indole ring of tryptophan with a halogen atom.

The enzyme RebH preferably originates from the organism Lechevalieria aerocolonigenes.

RebF: flavin reductase (EC 1.5.1.30)

FAD+NADH+H⁺→FADH₂+NAD⁺.

The flavin reductase reduces FAD to FADH₂.

The enzyme RebF preferably originates from the organism Lechevalieria aerocolonigenes.

INMT: indole N-methyltransferase (EC 2.1.1.49)

S-adenosyl-L-methionine+an amine→S-adenosyl-L-homocysteine+a methylated amine.

The indole N-methyltransferase catalyzes consecutive N-methylation steps of tryptamine.

The enzyme INMT preferably originates from the organism Oryctolagus cuniculus.

Halogenated: the term herein refers to a compound or a molecule with one or more halogen atoms introduced in the place of hydrogen, i.e. a compound substituted with one or more halogen atoms. If one halogen atom is present, the compound is halogenated or monohalogenated; if two halogen atoms are present, the compound is dihalogenated; if three halogen atoms are present, the compound is trihalogenated. In the context of the present disclosure, the halogen atom(s) may be present in position 2, 5, 6 and/or 7.

Halogenated tryptophan derivatives: the term herein refers to compounds obtainable from halogenated tryptophan, such as halogenated tryptamine, halogenated N-methyltryptamine, halogenated N,N-dimethyltryptamine and halogenated N,N,N-trimethyltryptamine.

Corresponding halogenated compound: the term herein refers to a halogenated compound derived from another halogenated compound by the action of an enzyme, e.g. a tryptophan decarboxylase, such as CrTDC, or an indole N-methyltransferase, such as OcINMT. Such a halogenated compound contains the same halogen atoms in the same positions of the indole ring as the halogenated compound from which it is derived. For example, the corresponding halogenated tryptamine of 5,6-dichlorotryptophan is 5,6-dichlorotryptamine and the corresponding halogenated N,N-dimethyltryptamine of 5-chlorotryptamine is 5-chloro-N,N-dimethyltryptamine.

Titer: the term herein refers to the concentration of a compound that accumulates inside the production host and/or in the extracellular media during cultivation of the host.

Mutation: the term herein refers to a change in nucleic acid sequence compared to the parent nucleic acid sequence. The term mutation covers single nucleotide mutations, but also insertions and deletions, i.e. any change that leads to a different nucleic acid sequence than the parent nucleic acid sequence. The term mutation thus encompasses deletions, such as deletions of a whole gene or coding sequence.

“Identity”, “similarity” and “homology” with respect to a polynucleotide (or polypeptide) is defined herein as the percentage of nucleic acids (or amino acids) in the candidate sequence that are identical with the residues of a corresponding native nucleic acids (or amino acids), after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985) 150: 1-5) as part of the sequence identity. Neither 5′ or 3′ extensions nor insertions result in a reduction of identity, similarity or homology. Methods and computer programs for the alignments are well known in the art.

Yeast Cell

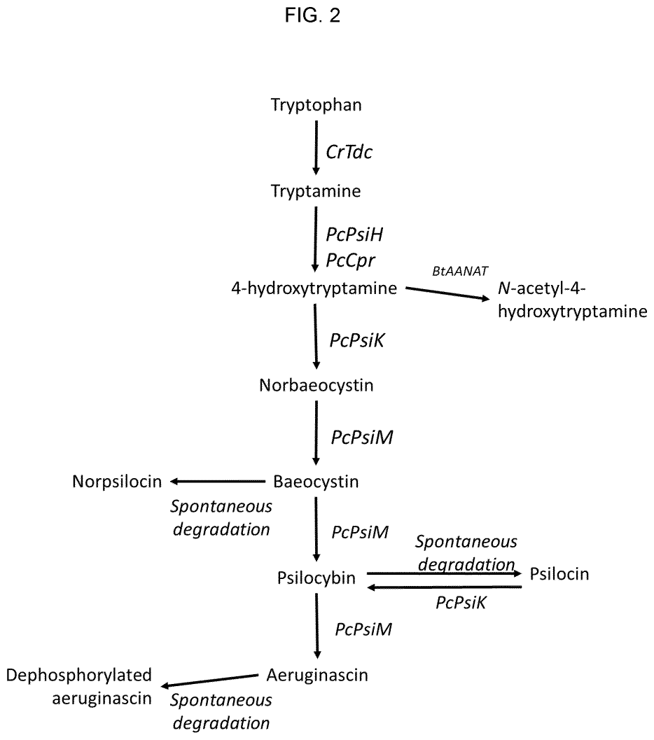

The present disclosure relates to a yeast cell capable of producing 4-hydroxytryptamine and optionally derivatives thereof, as outlined in FIG. 2. The inventors have found that heterologous expression of a tryptophan decarboxylase, a tryptamine 4-monooxygenase and a cytochrome P450 reductase in yeast results in the production of 4-hydroxytryptamine. Other derivatives can also be obtained by expressing additional enzymes in the yeast cell, as described in detail herein. The yeast cell can be engineered as described herein; the nature of the desired product will dictate which enzymes should be introduced in the yeast cell, as illustrated in FIG. 2. The yeast cell is preferably non-naturally occurring.

In one aspect, the present invention provides a yeast cell capable of producing 4-hydroxytryptamine and optionally derivatives thereof, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,

wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell, a Saccharomyces boulardii cell or a Yarrowia lipolytica cell.

Throughout the present disclosure, it will be understood that the cells can produce the compounds of interest listed herein when incubated in a cultivation medium under conditions that enable the cell to grow and produce the desired compound. From the description of the production host cells provided herein, the skilled person will not have difficulties in identifying suitable cultivation media and conditions to achieve production.

Production of 4-Hydroxytryptamine

The yeast cell of the present disclosure can produce 4-hydroxytryptamine. This requires that the yeast cell expresses a tryptophan decarboxylase capable of converting tryptophan to tryptamine, a tryptamine 4-monooxygenase and a cytochrome P450 reductase which together are capable of converting tryptamine to 4-hydroxytryptamine.

In some embodiments, the tryptophan decarboxylase (EC 4.1.1.105) is a heterologous tryptophan decarboxylase. In some embodiments, the tryptophan decarboxylase is CrTDC (SEQ ID NO: 1) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine.

In some embodiments, the tryptamine 4-monooxygenase (EC 1.14.99.59) is a heterologous tryptamine 4-monooxygenase. In some embodiments, the tryptamine 4-monooxygenase is PcPsiH (SEQ ID NO: 2) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the cytochrome P450 reductase (EC 1.6.2.4) is a heterologous cytochrome P450 reductase. In some embodiments, the cytochrome P450 reductase is PcCpr (SEQ ID NO: 3) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses CrTDC, PcPsiH and PcCpr as set forth in SEQ ID NO: 1, SEQ ID NO: 2 and SEQ ID NO: 3, respectively, or functional variants thereof having at least 85% homology thereto.

In some embodiments, the yeast cell is capable of producing 4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

The yeast cell may be further engineered to allow production of 4-hydroxytryptamine derivatives, as described herein below in detail. Such derivatives include norbaeocystin, N-acetyl-4-hydroxytryptamine, baeocystin, psilocybin, psilocin and aeruginascin. Norpsilocin is another 4-hydroxytryptamin derivative which may be obtained by spontaneous degradation of baeocystin. Hence, a yeast cell capable of producing baeocystin may also produce norpsilocin, in particular by spontaneous degradation. Psilocin may be obtained from psilocyin by spontaneous degradation.

Hence, a yeast cell capable of producing psilocybin may also produce psilocin. Aeruginascin may be spontaneously converted to dephosphorylated aeruginascin. Hence, a yeast cell capable of producing aeruginascin may also produce dephosphorylated aeruginascin. FIG. 2 provides an overview of the products that can be obtained using the present yeast cells, depending on which enzymes are expressed.

In some embodiments, the yeast cell further expresses a cytochrome b5, such as a heterologous cytochrome b5. In some embodiments, the cytochrome b5 is the putative P. cubensis cytochrome b5 as set forth in SEQ ID NO: 42, or a functional variant thereof having at least 80% homology or identity thereto and retaining the cytochrome b5 function, such as a functional variant having at least 85%, such as at least 90%, such as at least 95%, such as at least 96%, such as at least 97%, such as at least 98%, or such as at least 99% homology or identity to SEQ ID NO: 42. In some embodiments the cytochrome b5 is encoded by SEQ ID NO: 43 or a homologue thereof having at least 80% homology or identity thereto, such as at least 85%, such as at least 90%, such as at least 95%, such as at least 96%, such as at least 97%, such as at least 98%, or such as at least 99% homology or identity to SEQ ID NO: 43. Such homologues encode a protein which preferably retains the function of the original protein.

Production of Norbaeocystin

The yeast cell may be further engineered to produce norbaeocystin from 4-hydroxytryptamine. This can be achieved by introducing a 4-hydroxytryptamine kinase capable of converting 4-hydroxytryptamine to norbaeocystin.

In some embodiments, the yeast cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin, whereby the yeast cell is capable of converting 4-hydroxytryptamine to norbaeocystin. The yeast cell can thus produce norbaeocystin.

In some embodiments, the 4-hydroxytryptamine kinase (EC 2.7.1.222) is a heterologous 4-hydroxytryptamine kinase. In some embodiments, the 4-hydroxytryptamine kinase is PcPsiK (SEQ ID NO: 4) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the yeast cell is capable of producing norbaeocystin and expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto, wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine; and
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin, whereby the yeast cell is capable of converting 4-hydroxytryptamine to norbaeocystin.

In some embodiments, the yeast cell expresses CrTDC, PcPsiH and PcCpr as set forth in SEQ ID NO: 1, SEQ ID NO: 2 and SEQ ID NO: 3, respectively, or functional variants thereof having at least 85% homology thereto, and further expresses PcPsiK as set forth in SEQ ID NO: 4 or a functional variant thereof having at least 85% homology thereto.

In some embodiments, the yeast cell is capable of producing norbaeocystin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Production of Baeocystin and Norpsilocin

The yeast cell may be further engineered to produce baeocystin from norbaeocystin. This can be achieved by introducing a norbaeocystin N-methyl transferase (also termed psilocybin synthase; the two terms are herein used interchangeably) capable of converting norbaeocystin to baeocystin.

In some embodiments, the yeast cell further expresses a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the yeast cell is capable of converting norbaeocystin to baeocystin. The yeast cell can thus produce baeocystin.

In some embodiments, the norbaeocystin N-methyl transferase/psilocybin synthase (EC 2.1.1.345) is a heterologous norbaeocystin N-methyl transferase/psilocybin synthase. In some embodiments, the norbaeocystin N-methyl transferase/psilocybin synthase is PcPsiM (SEQ ID NO: 5) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the yeast cell is capable of producing baeocystin and expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto, wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin, and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the yeast cell is capable of converting norbaeocystin to baeocystin.

In some embodiments, the yeast cell expresses CrTDC, PcPsiH, PcCpr and PcPsiK as set forth in SEQ ID NO: 1, SEQ ID NO: 2, SEQ ID NO: 3 and SEQ ID NO: 4, respectively, or functional variants thereof having at least 85% homology thereto, and further expresses PcPsiM as set forth in SEQ ID NO: 5 or a functional variant thereof having at least 85% homology thereto.

In some embodiments, the yeast cell is capable of producing baeocystin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

The baeocystin produced by the cell may be further converted to norpsilocin. This may happen spontaneously, by spontaneous degradation of baeocystin to norpsilocin.

In some embodiments, the yeast cell is capable of producing norpsilocin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Production of Psilocybin and Psilocin

The yeast cell may be further engineered to produce psilocybin. This can be done by expressing a norbaeocystin N-methyl transferase/psilocybin synthase (EC 2.1.1.345) in the yeast cell, which enzyme is capable of converting norbaeocystin to baeocystin and further to psilocybin. The resulting yeast cell can thus produce psilocybin. Psilocin may be produced as a result of the action of phosphatases in the cell, or by spontaneous degradation. Thus in some embodiments the yeast cell is capable of producing psilocin.

Accordingly, in some embodiments, the yeast cell expresses a norbaeocystin N-methyl transferase/psilocybin synthase (EC 2.1.1.345) as described above, preferably a heterologous psilocybin synthase such as PcPsiM (SEQ ID NO: 5) or a functional variant thereof having at least 80% homology, such as at least 85%, such as at least 90%, such as at least 95% homology thereto, wherein the psilocybin synthase is capable of converting norbaeocystin to psilocybin, whereby the yeast cell is capable of converting norbaeocystin to psilocybin.

Accordingly, in some embodiments, the yeast cell is capable of producing psilocybin and expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto, wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin, and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and further to psilocybin, whereby the yeast cell is capable of converting norbaeocystin to psilocybin.

In some embodiments, the yeast cell expresses CrTDC, PcPsiH, PcCpr and PcPsiK as set forth in SEQ ID NO: 1, SEQ ID NO: 2, SEQ ID NO: 3 and SEQ ID NO: 4, respectively, or functional variants thereof having at least 85% homology thereto, and further expresses PcPsiM as set forth in SEQ ID NO: 5 or a functional variant thereof having at least 85% homology thereto.

In some embodiments, the norbaeocystin N-methyl transferase/psilocybin synthase (EC 2.1.1.345) is a heterologous psilocybin synthase. In some embodiments, the psilocybin synthase is PcPsiM (SEQ ID NO: 5) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell is capable of producing psilocybin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the psilocybin is converted to psilocin and the yeast cell is capable of producing psilocin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

If it is desirable to direct the production towards psilocin, it may be advantageous to modulate the activity of the 4-hydroxytryptamine kinase. As is shown on FIG. 2, this is because this enzyme is capable of converting psilocin back to psilocybin. Accordingly, variants of this enzyme which have reduced capability of converting psilocin to psilocybin may be particularly advantageous for psilocin production, for example variants which can still fully catalyse the conversion of 4-hydroxytryptamine to norbaeocystin.

Production of Aeruqinascin and Dephosphorylated Aeruqinascin

Psilocybin can be further converted to aeruginascin by the action of the norbaeocystin N-methyl transferase/psilocybin synthase. This can be done by expressing a norbaeocystin N-methyl transferase/psilocybin synthase (EC 2.1.1.345) in the yeast cell, which enzyme is capable of converting psilocybin to aeruginascin. The resulting yeast cell can thus produce aeruginascin. Aeruginascin may then be spontaneously dephosphorylated, yielding dephosphorylated aeruginascin. Thus in some embodiments the yeast cell is capable of producing aeruginascin and optionally dephosphorylated aeruginascin.

Accordingly, in some embodiments, the yeast cell expresses a norbaeocystin N-methyl transferase/psilocybin synthase (EC 2.1.1.345) as described above, preferably a heterologous psilocybin synthase such as PcPsiM (SEQ ID NO: 5) or a functional variant thereof having at least 80% homology, such as at least 85%, such as at least 90%, such as at least 95% homology thereto, wherein the psilocybin synthase is capable of converting psilocybin to aeruginascin, whereby the yeast cell is capable of converting psilocybin to aeruginascin.

Accordingly, in some embodiments, the yeast cell is capable of producing aeruginascin and expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome

P450 reductase such as PcCpr (SEQ ID NO: 3) or a functional variant thereof having at least 80% homology, such as at least 85%, such as at least 90%, such as at least 95% homology thereto,

wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin, and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and further to psilocybin, and of converting psilocybin to aeruginascin, whereby the yeast cell is capable of producing aeruginascin, and optionally dephosphorylated aeruginascin.

In some embodiments, the yeast cell expresses CrTDC, PcPsiH, PcCpr and PcPsiK as set forth in SEQ ID NO: 1, SEQ ID NO: 2, SEQ ID NO: 3 and SEQ ID NO: 4, respectively, or functional variants thereof having at least 85% homology thereto, and further expresses PcPsiM as set forth in SEQ ID NO: 5 or a functional variant thereof having at least 85% homology thereto.

In some embodiments, the norbaeocystin N-methyl transferase/psilocybin synthase (EC 2.1.1.345) is a heterologous psilocybin synthase. In some embodiments, the psilocybin synthase is PcPsiM (SEQ ID NO: 5) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell is capable of producing aeruginascin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the aeruginascin is dephosphorylated and the yeast cell is capable of producing dephosphorylated aeruginascin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Production of N-Acetyl-4-Hydroxytryptamine

The yeast cell may be further engineered to produce N-acetyl-4-hydroxytryptamine from 4-hydroxytryptamine. This can be achieved by introducing a serotonin N-acetyltransferase (EC 2.3.1.87) capable of converting 4-hydroxytryptamine to N-acetyl-4-hydroxytryptamine.

In some embodiments, the yeast cell further expresses a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine, whereby the yeast cell is capable of converting 4-hydroxytryptamine to N-acetyl-4-hydroxytryptamine. The yeast cell can thus produce N-acetyl-4-hydroxytryptamine.

In some embodiments, the serotonin N-acetyltransferase (EC 2.3.1.87) is a heterologous serotonin N-acetyltransferase. In some embodiments, the serotonin N-acetyltransferase is BtAANAT (SEQ ID NO: 11) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the yeast cell is capable of producing N-acetyl-4-hydroxytryptamine and expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto, wherein the tryptamine 4-monooxygenase and the cytochrome P450 reductase together catalyse the conversion of tryptamine to 4-hydroxytryptamine, whereby the yeast cell is capable of converting tryptophan to 4-hydroxytryptamine; and
a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine, whereby the yeast cell is capable of converting 4-hydroxytryptamine to N-acetyl-4-hydroxytryptamine.

In addition to the enzymes necessary for producing N-acetyl-4-hydroxytryptamine, the yeast cell may also express the enzymes necessary for producing norbaeocystin, and optionally baeocystin and psilocybin, as described herein above.

In some embodiments, the yeast cell expresses CrTDC, PcPsiH and PcCpr as set forth in SEQ ID NO: 1, SEQ ID NO: 2 and SEQ ID NO: 3, respectively, or functional variants thereof having at least 85% homology thereto, and further expresses BtAANAT as set forth in SEQ ID NO: 11 or a functional variant thereof having at least 85% homology thereto.

In some embodiments, the yeast cell is capable of producing N-acetyl-4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Expression of Heterologous Enzymes

In some embodiments, one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is under the control of an inducible promoter.

In some embodiments, one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is codon-optimised for the yeast cell, as is known in the art.

In some embodiments, one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is present in 2 to 30 copies.

In some embodiments, one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is integrated in the genome of the yeast cell.

In some embodiments, one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is expressed from a vector such as a plasmid.

In some embodiments, expression of one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase can be induced or repressed, for instance to obtain transient expression, as is known in the art.

Nucleic acid constructs useful for obtaining yeast cells capable of producing 4-hydroxytryptamine or derivatives thereof are described in the section “Nucleic acid construct”.

### Other Modifications

Because the present pathways require tryptophan as a first substrate, and without being bound by theory, it may be advantageous to modify the yeast cell in such a manner that tryptophan metabolism is directed towards increased tryptophan synthesis, thereby further increasing the titers of 4-hydroxytryptamine or derivatives thereof.

In some embodiments, the yeast cell further comprises one or more mutations resulting in increased availability of L-tryptophan.

In some embodiments, the one or more mutations is in one or more genes encoding a transcriptional repressor(s) of genes of the aromatic amino acid precursor pathway; the mutation may be in the coding region or in the promoter of the gene. In Saccharomyces cerevisiae, examples of such genes are ARO1, ARO2, ARO3 or ARO4. Mutations, including deletions, inactivating or partially inactivating the products of such genes may increase tryptophan availability and help further improve the titers. In some embodiments, the one or more mutations is a mutation resulting in partial or total loss of activity of the one or more transcriptional repressor(s). In some embodiments, the transcriptional repressor is Ric1 and the RIC1 gene is mutated in such a way that Ric1 function is abolished, e.g. by a deletion.

Alternatively or additionally, genes involved in tryptophan biosynthesis may be mutated or overexpressed. In S. cerevisiae, examples of such genes are TRP1, TRP2, TRP3, TRP4 or TRP5. Mutations conferring increased activity may be particularly advantageous. The genes may also be overexpressed as is known in the art, for example by taking advantage of a constitutive promoter.

In some embodiments, the yeast cell is a S. cerevisiae cell and the cell expresses a mutated ARO4 gene, where the mutation removes feedback regulation. The mutation may be in the coding region or in the promoter of the gene. In a specific embodiment, the cell expresses an Aro4 mutant having a mutation at position 229, such as a K229L substitution. The wild type sequence of ARO4 is set forth in SEQ ID NO: 12.

In some embodiments, the yeast cell overexpresses genes involved in the shikimate pathway, such as ARO1 and/or ARO2. The sequences of ARO1 and ARO2 are as set forth in SEQ ID NO: 13 and SEQ ID NO: 14, respectively.

In some embodiments, the yeast cell comprises mutations which increase the flux towards the shikimate pathway. For example, the yeast cell is S. cerevisiae and CDC19 (SEQ ID NO: 26) is mutated, where the mutation leads to a partial or total loss of activity. The mutation may be in the coding region or in the promoter of the gene.

In some embodiments, the yeast cell has a mutation, for example a deletion, of genes involved in tryptophan catabolism; the mutation may be in the coding region or in the promoter of the gene. For example, the yeast cell is a S. cerevisiae cell and ARO8 and/or ARO9 are deleted or mutated, where the mutation leads to a loss of function, to prevent tryptophan degradation. The sequences of ARO8 and ARO9 are set forth in SEQ ID NO: 23 and SEQ ID NO: 24, respectively.

In some embodiments, the yeast cell comprises mutations which direct the glutamine flux towards tryptophan. For example, the yeast cell comprises a mutation or a deletion of GLT1 (SEQ ID NO: 25). In some embodiments, the yeast cell is cultivated in a medium which is supplemented with glutamine. The mutation may be in the coding region or in the promoter of the gene.

In some embodiments, the yeast cell is a S. cerevisiae cell and the cell expresses a mutated TRP2 gene, where the mutation removes feedback regulation. In a specific embodiment, the cell expresses a Trp2 mutant having a mutation at position 65 and/or 76, such as an S65R and/or an S76L substitution. The wild type sequence of TRP2 is set forth in SEQ ID NO: 16. The mutation may be in the coding region or in the promoter of the gene.

In some embodiments, the yeast cell overexpresses one or more genes involved in the tryptophan synthesis pathway, such as TRP1, TRP2, TRP3, TRP4 and/or TRP5, the sequences of which are set forth in SEQ ID NO: 15, SEQ ID NO: 16, SEQ ID NO: 17, SEQ ID NO: 18 and SEQ ID NO: 19, respectively.

In some embodiments, the yeast cell overexpresses one or more genes involved in the serine pathway, such as SER1, SER2, SER3 and/or SER33, the sequences of which are set forth in SEQ ID NO: 20, SEQ ID NO: 21, SEQ ID NO: 22 and SEQ ID NO: 31, respectively.

In some embodiments, the yeast cell comprises modifications which result in increased NADPH availability. For example, the yeast cell is S. cerevisiae and one or more of STB5 (SEQ ID NO: 26), POS5 (SEQ ID NO: 27) and ZWF1 (SEQ ID NO: 29) are overexpressed.

In the psilocybin biosynthetic pathway, the conversion of tryptamine to 4-hydroxytryptamine is catalyzed by the CYP PcPsiH. The catalytic cycle of monooxygenation by a CYP proceeds as follows: the CYP binds its substrate in the active site adjacent to the CYP heme co-factor, which is in ferric state. The partner

CPR supplies an electron from NADPH, which reduces the heme-complex to the ferrous state. This reduction enables the heme-complex to bind molecular oxygen, forming a ferric superoxo state. Input of a second electron is then required, which leads to cleavage of the O—O bond, and subsequently product formation. CPRs are capable of supplying this second electron, but if it is not supplied fast enough, the superoxide anion in the ferric superoxo complex is released, a process termed uncoupling.

Uncoupling of a CYP catalyzed reaction results in lack of product formation and release of superoxide anion that dismutates to hydrogen peroxide, which can be detrimental to growth.

Cytochrome b5 are a class of membrane-bound heme-proteins that often take part in CYP catalyzed reactions. Without being bound by theory, it is thought that cytochrome b5 is capable of providing a rapid input of the second electron (from NADH) in the catalytic cycle, thereby decreasing uncoupling of the CYP catalyzed reaction and increasing product formation.

In some embodiments, any of the yeast strains described herein further expresses a cytochrome b5, such as a heterologous cytochrome b5. In some embodiments, the cytochrome b5 is the putative P. cubensis cytochrome b5 as set forth in SEQ ID NO: 43, or a functional variant thereof having at least 80% homology or identity thereto and retaining the cytochrome b5 function, such as a functional variant having at least 85%, such as at least 90%, such as at least 95%, such as at least 96%, such as at least 97%, such as at least 98%, or such as at least 99% homology or identity to SEQ ID NO: 43. In some embodiments the cytochrome b5 is encoded by SEQ ID NO: 42 or a homologue thereof having at least 80% homology or identity thereto, such as at least 85%, such as at least 90%, such as at least 95%, such as at least 96%, such as at least 97%, such as at least 98%, or such as at least 99% homology or identity to SEQ ID NO: 42. Such homologues encode a protein which preferably retains the function of the original protein.

Without being bound by theory, modifications which increase availability of S-adenosylmethionine (SAM) may further help improve the titers of the tryptamine derivatives obtainable by the methods disclosed herein. In some embodiments, the cell is modified, for example the ergosterol biosynthetic pathway is modified. In embodiments where the cell is a yeast cell, the modification can be a mutation in or a deletion of the gene encoding Erg4 (SEQ ID NO: 44), resulting in a partial or total loss of Erg4. The gene encoding Erg4 is set forth in SEQ ID NO: 45. The mutation may be in the coding region or in the promoter of the gene.

In other embodiments, the gene encoding an S-adenosylmethionine decarboxylase proenzyme is mutated or deleted so as to result in a partial or total loss of activity. S-adenosylmethionine decarboxylase catalyzes the decarboxylation of SAM into S-adenosylmethioninamine as the first step of the spermidine biosynthesis. In embodiments where the cell is a yeast cell, the modification can be a mutation in or a deletion of the gene encoding Spe2 (SEQ ID NO: 46), resulting in a partial or total loss of Spe2. The gene encoding Spe2 is set forth in SEQ ID NO: 47. The mutation may be in the coding region or in the promoter of the gene.

In some embodiments, the above modifications are combined. For example, in embodiments where the cell is a yeast cell, the yeast cell has reduced activity of Erg4 or Spe 2 (or both), and further has reduced activity of Ric1 as described herein above. ARO1 and ARO2 may also be overexpressed in such strains, which may in addition have any of the modifications described herein above.

Examples of Useful Yeast Cells

In this section a number of specific yeast cells representing specific embodiments are listed. In some embodiments, the cell is a Saccharomyces cerevisiae cell or a Saccharomyces boulardii cell. In other embodiments, the cell is a Yarrowia lipolytica cell.

In a specific embodiment, the yeast cell is capable of producing 4-hydroxytryptamine, and expresses:
CrTDC (SEQ ID NO: 1);
PcPsiH (SEQ ID NO: 2);
PcCpr (SEQ ID NO: 3); and
PcPsiK (SEQ ID NO: 4),

Or functional variants thereof having at least 85% homology thereto. Preferably the yeast cell is a S. cerevisiae cell or a Y. lipolytica cell.

In another embodiment, the yeast cell is capable of producing norbaeocystin, and expresses:
CrTDC (SEQ ID NO: 1);
PcPsiH (SEQ ID NO: 2);
PcCpr (SEQ ID NO: 3); and
PcPsiK (SEQ ID NO: 4),

Or functional variants thereof having at least 85% homology thereto. Preferably the yeast cell is a S. cerevisiae cell or a Y. lipolytica cell.

In another embodiment, the yeast cell is capable of baeocystin and/or psilocybin and/or psilocin and/or aeruginascin and/or dephosphorylated aeruginascin, and expresses:
CrTDC (SEQ ID NO: 1);
PcPsiH (SEQ ID NO: 2);
PcCpr (SEQ ID NO: 3);
PcPsiK (SEQ ID NO: 4); and
PcPsiM (SEQ ID NO: 5),

Or functional variants thereof having at least 85% homology thereto. Preferably the yeast cell is a S. cerevisiae cell or a Y. lipolytica cell.

In some embodiments, the yeast cell further expresses BtAANAT (SEQ ID NO: 5) or a functional variant thereof having at least 85% homology thereo, and is capable of producing N-acetyl-4-hydroxytryptamine. Preferably the yeast cell is a S. cerevisiae cell or a Y. lipolytica cell.

In some embodiments, one or more of the genes encoding enzymes of the aromatic amino acid precursor pathway ARO1, ARO2, TRP1, TRP3, TRP4, TRP5 have been overexpressed in the yeast cell. In some embodiments, the transcriptional repressor RIC1 is mutated and has reduced activity.

In some embodiments, the yeast cell further expresses a cytochrome b5 such as PcCyb5 as set forth in SEQ ID NO: 43.

Other Organisms

Other organisms besides yeast may also be useful as production organisms according to the present disclosure. Thus, in some embodiments the production cell is a microorganism or a plant cell. The microorganism may e.g. be a fungus or a bacteria.

Useful fungi include a fungus belonging to the genus of Aspergillus, e.g. A. niger, A. awamori, A. oryzae, A. nidulans, a yeast belonging to the genus of Saccharomyces, e.g. S. cerevisiae, S. kluyveri, S. bayanus, S. exiguus, S. sevazzi, S. uvarum, S. boulardii, a yeast belonging to the genus Kluyveromyces, e.g. K. lactis, K. marxianus var. marxianus, K. thermotolerans, a yeast belonging to the genus Candida, e.g. C. utilis C. tropicalis, C. albicans, C. lipolytica, C. versatilis, a yeast belonging to the genus Pichia, e.g. P. stipidis, P. pastoris, P. sorbitophila, other yeast genera such as Cryptococcus (e.g. C. aerius), Debaromyces (e.g. D. hansenii), Hansenula, Yarrowia (e.g. Y. lipolytica), Zygosaccharomyces (e.g. Z. bailii), Torulaspora (e.g. T. delbrueckii), Schizosaccharomyces (e.g. S. pombe), Brettanomyces (e.g. B. bruxellensis), Penicillium, Rhizopus, Fusarium, Fusidium, Gibberella, Mucor, Mortierella, or Trichoderma.

Useful bacteria include bacteria belonging to the genus Bacillus (e.g. B. subtilis), a species belonging to the genus Escherichia (e.g. E. coli), a species belonging to the genus Lactobacillus (e.g. L. casei), a species belonging to the genus Lactococcus (e.g. L. lactis), a species belonging to the genus Corynebacterium (e.g. C. glutamicum), a species belonging to the genus Acetobacter, a species belonging to the genus Acinetobacter, a species belonging to the genus Pseudomonas (e.g. P. putida), and a species belonging to the genus Streptomyces (e.g. S. coelicolor).

Useful plants include plants belonging to the genus Arabidopsis (e.g. A. thaliana), a species belonging to the genus Zea (e.g. Z. mays), a species belonging to the genus Medicago (e.g. M. truncatula), a species belonging to the genus Nicotiana (e.g. N. tabacum) and a species belonging to the genus Glycine (e.g. G. Max).

Methods of Production of 4-Hydroxytryptamine and Derivatives Thereof

The present disclosure relates to methods for producing 4-hydroxytryptamine and derivatives thereof. The yeast cells and nucleic acid constructs described herein are useful for microbial-based production of 4-hydroxytryptamine and derivatives thereof, including psilocybin. Throughout the present disclosure, it will be understood that the cells can produce the compounds of interest listed herein when incubated in a cultivation medium under conditions that enable the cell to grow and produce the desired compound. From the description of the production host cells provided herein, and knowing the type of host cell used, the skilled person will not have difficulties in identifying suitable cultivation media and conditions to achieve production. In particular, the cultivation may be performed aerobically or anaerobically, at temperatures and at pH suitable for supporting growth of the cell. The cultivation medium should include the required nutrients, and may be supplemented with precursors as applicable. The time of cultivation will vary depending on which cell is used, but can easily be adapted by the skilled person.

In one aspect, the present invention provides a method of producing 4-hydroxytryptamine and optionally derivatives thereof in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

Yeast cells useful for producing 4-hydroxytryptamine are described herein, in particular in the section “Production of 4-hydroxytryptamine” herein above.

Herein are also provided methods for producing derivatives of 4-hydroxytryptamine.

Norbaeocystin

In some embodiments, the 4-hydroxytryptamine derivative is norbaeocystin. In such embodiments, the yeast cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto. Yeast cells useful for production of norbaeocystin are described herein, in particular in the section “Production of norbaeocystin” herein above.

In some embodiments, the method is for producing norbaeocystin and the yeast cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.

In some embodiments, the present invention provides a method of producing norbaeocystin in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto; and
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.

Baeocystin and Norpsilocin

In some embodiments, the 4-hydroxytryptamine derivative is baeocystin. In such embodiments, the yeast cell further expresses a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto. The baeocystin may be further converted to norpsilocin. Hence in some embodiments the method results in production of norpsilocin. Yeast cells useful for production of baeocystin are described herein, in particular in the section “Production of baeocystin and norpsilocin” herein above.

In some embodiments, the present invention provides a method of producing baeocystin and optionally norpsilocin in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto; and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the yeast cell is capable of converting norbaeocystin to baeocystin and optionally to norpsilocin.

Psilocybin and Psilocin

In some embodiments, the 4-hydroxytryptamine derivative is psilocybin. In such embodiments, the yeast cell expresses a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto. Yeast cells useful for production of psilocybin are described herein, in particular in the section “Production of psilocybin and psilocin” herein above.

In some embodiments, the present invention provides a method of producing psilocybin and optionally psilocin in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto; and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and further to psilocybin and optionally to psilocin, whereby the yeast cell is capable of converting norbaeocystin to psilocybin and optionally to psilocin.

The psilocybin may be further converted spontaneously to psilocin. Hence in some embodiments the method results in production of psilocin.

Aeruginascin and Dephosphorylated Aeruginascin

The psilocybin may also be further converted to aeruginascin by the action of the norbaeocystin N-methyl transferase/psilocybin synthase. Hence in some embodiments the methods are for production of aeruginascin, which may optionally be spontaneously converted to dephosphorylated aeruginascin. Yeast cells useful for production of psilocybin are described herein, in particular in the section “Production of aeruginascin and dephosphorylated aeruginascin” herein above.

In some embodiments, the present invention provides a method of producing aeruginascin and optionally dephosphorylated aeruginascin in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto; and
a norbaeocystin N-methyl transferase/psilocybin synthase (EC 2.1.1.345), preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and further to psilocybin and to aeruginascin, whereby the yeast cell is capable of converting norbaeocystin to psilocybin and to aeruginascin, whereby the yeast cell produces aeruginascin and/or dephosphorylated aeruginascin.

N-Acetyl-4-Hydroxytryptamine

In some embodiments, the 4-hydroxytryptamine derivative is N-acetyl-4-hydroxytryptamine. In such embodiments, the yeast cell expresses a serotonin N-acetyltransferase (EC 2.3.1.87) capable of converting 4-hydroxytryptamine to N-acetyl-4-hydroxytryptamine, preferably a heterologous norbaeocystin N-methyl transferase/psilocybin synthase such as PcPsiM (SEQ ID NO: 5) or a functional variant thereof having at least 80% homology, such as at least 85%, such as at least 90%, such as at least 95% homology thereto. Yeast cells useful for production of psilocybin are described herein, in particular in the section “Production of N-acetyl-4-hydroxytryptamine” herein above.

In some embodiments, the present invention provides a method of producing N-acetyl-4-hydroxytryptamine in a yeast cell, said method comprising the steps of providing a yeast cell and incubating said yeast cell in a medium, wherein the yeast cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto; and
a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine, whereby the yeast cell is capable of converting 4-hydroxytryptamine to N-acetyl-4-hydroxytryptamine.

Such yeast cells may also express, in addition to the above, a 4-hydroxytryptamine kinase and optionally a norbaeocystin N-methyl transferase/psilocybin synthase as described herein above.

Useful Enzymes for Production of 4-Hydroxytryptamine and Derivatives Thereof

Enzymes useful for the present methods, which can advantageously be introduced in the yeast cell, are described in detail herein above in the section entitled “Yeast cell”.

In some embodiments, the tryptophan decarboxylase (EC 4.1.1.105) is a heterologous tryptophan decarboxylase such as CrTDC (SEQ ID NO: 1) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine.

In some embodiments, the tryptamine 4-monooxygenase (EC 1.14.99.59) is a heterologous tryptamine 4-monooxygenase such as PcPsiH (SEQ ID NO: 2) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the cytochrome P450 reductase (EC 1.6.2.4) is a heterologous cytochrome P450 reductase such as PcCpr (SEQ ID NO: 3) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the 4-hydroxytryptamine kinase (EC 2.7.1.222) is a heterologous 4-hydroxytryptamine kinase such as PcPsiK (SEQ ID NO: 4) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the norbaeocystin N-methyl transferase/psilocybin synthase (EC 2.1.1.345) is a heterologous psilocybin synthase such as PcPsiM (SEQ ID NO: 5) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the serotonin N-acetyltransferase (EC 2.3.1.87) is a heterologous serotonin N-acetyltransferase such as BtAANAT (SEQ ID NO: 11) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the serotonin N-acetyltransferase is capable of converting 4-hydroxytryptamine to N-acetyl-4-hydroxytryptamine.

In some embodiments, the medium comprises tryptophan and/or the yeast cell is capable of synthesising tryptophan.

The yeast cell may further comprise any of the modifications detailed in the section “Other modifications”. For example, in some embodiments, the yeast cell further comprises one or more mutations resulting in increased availability of L-tryptophan.

Recovering the 4-Hydroxytryptamine and/or Derivatives Thereof

The present methods may comprise a further step of recovering the compounds obtained by the methods disclosed herein. Methods for recovering the products obtained by the present invention are known in the art, for example organic solvent extraction followed by lyophilisation and purification by preparative HPLC or similar column purification techniques. For example, the step of recovering the compound(s) may comprise separating the cell culture in a solid phase and in a liquid phase to obtain a supernatant. The supernatant can then be contacted with one or more adsorbent resins to which the compound(s) can bind, and the compound(s) can then be eluted as is known in the art. Alternatively, one or more ion exchange or reversed-phase chromatography columns can be used. Another option is to employ liquid-liquid extraction in an immiscible solvent, which may optionally be evaporated before precipitating the compound(s), or further liquid-liquid extraction may be employed.

The yeast cell is preferably as defined herein.

In some embodiments, the method is for production of 4-hydroxytryptamine and further comprises a step of recovering the 4-hydroxytryptamine.

In some embodiments, the method is for production of norbaeocystin and further comprises a step of recovering the norbaeocystin.

In some embodiments, the method is for production of baeocystin and further comprises a step of recovering the baeocystin. Optionally, the method is for production of norpsilocin and further comprises a step of recovering the norpsilocin.

In some embodiments, the method is for production of psilocybin and optionally psilocin and further comprises a step of recovering the psilocybin and optionally the psilocin.

In some embodiments, the method is for production of aeruginascin and optionally dephosphorylated aeruginascin and further comprises a step of recovering the aeruginascin and optionally the dephosphorylated aeruginascin.

In some embodiments, the method is for production of N-acetyl-4-hydroxytryptamine and further comprises a step of recovering the N-acetyl-4-hydroxytryptamine.

Titers

The present methods are useful for producing 4-hydroxytryptamine and derivatives thereof with high titers.

In some embodiments, 4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, norbaeocystin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, baeocystin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, norpsilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, psilocybin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, psilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, aeruginascin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, dephosphorylated aeruginascin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, N-acetyl-4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Methods for determining the titer are known in the art, for example by measuring the peak area from LC-MS analysis and comparing to the peak area of an authentic analytical standard of known concentration.

The titer of any of the above can be increased by introducing further modifications in the cell, such as any of the modifications described in “Other modifications”. In some embodiments, the titers may also or alternatively be increased by supplementing the culture medium with glutamine. In some embodiments, the medium is supplemented with at least 1 g/L glutamine, such as at least 2 g/L, such as at least 3 g/L glutamine, such as at least 4 g/L glutamine, such as at least 5 g/L glutamine, such as at least 6 g/L glutamine, such as at least 7 g/L glutamine, such as at least 8 g/L glutamine, such as at least 9 g/L glutamine, such as at least 10 g/L glutamine, or more.

4-Hydroxytryptamine and Derivatives Thereof Obtainable by the Present Methods

In one aspect, the present invention provides 4-hydroxytryptamine and derivatives thereof obtainable by a method as disclosed herein.

In some embodiments, the present invention provides norbaeocystin obtainable by the method described herein. In some embodiments, the present invention provides baeocystin obtainable by the method described herein. In some embodiments, the present invention provides norpsilocin obtainable by the method described herein. In some embodiments, the present invention provides psilocybin obtainable by the method described herein. In some embodiments, the present invention provides psilocin obtainable by the method described herein. In some embodiments, the present invention provides aeruginascin obtainable by the method described herein. In some embodiments, the present invention provides dephosphorylated aeruginascin obtainable by the method described herein. In some embodiments, the present invention provides N-acetyl-4-hydroxytryptamine obtainable by the method described herein.

Nucleic Acid Constructs

Also provided herein are nucleic acid constructs useful for engineering a yeast cell capable of producing 4-hydroxytryptamine or derivatives thereof as described above. The present nucleic acid constructs may be provided as one or more nucleic acid molecules or polynucleotides, for example they may be comprised in one or more vectors. Such nucleic acids may be introduced in the cell by methods known in the art.

It will be understood that throughout the present disclosure, the term ‘nucleic acid encoding an activity’ shall refer to a nucleic acid molecule capable of encoding a peptide, a protein or a fragment thereof having said activity. Such nucleic acid molecules may be open reading frames or genes, or fragments thereof.

In one aspect, the present invention provides a nucleic acid construct for modifying a yeast cell, said construct comprising:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto; and
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

In some embodiments, the nucleic acid further comprises a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.

In some embodiments, the present invention provides a nucleic acid construct for modifying a yeast cell, said construct comprising:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto;
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto; and
a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.

In some embodiments, the nucleic acid further comprises a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.

In some embodiments, the present invention provides a nucleic acid construct for modifying a yeast cell, said construct comprising:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto;
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto; and
a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.

Any of the above nucleic acid constructs may further comprise a sixth polynucleotide encoding a serotonin N-acetyltransferase (EC 2.3.1.87) capable of converting 4-hydroxytryptamine to N-acetyl-4-hydroxytryptamine. In some embodiments, the serotonin N-acetyltransferase is a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine, whereby the yeast cell is capable of converting 4-hydroxytryptamine to N-acetyl-4-hydroxytryptamine. The yeast cell can thus produce N-acetyl-4-hydroxytryptamine.

Thus in some embodiments, the nucleic acid construct comprises:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto;
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto; and
a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto.

In other embodiments, the nucleic acid construct comprises:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto;
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto; and
a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto.

In some embodiments, the nucleic acid construct comprises:
a first polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto;
a second polynucleotide encoding a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto;
a third polynucleotide encoding a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto;
a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto;
a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto; and
a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto.

In some embodiments, the tryptophan decarboxylase (EC 4.1.1.105) is preferably a heterologous tryptophan decarboxylase such as CrTDC (SEQ ID NO: 1) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine.

In some embodiments, the tryptamine 4-monooxygenase (EC 1.14.99.59) is preferably a heterologous tryptamine 4-monooxygenase such as PcPsiH (SEQ ID NO: 2) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the cytochrome P450 reductase (EC 1.6.2.4) is preferably a heterologous cytochrome P450 reductase such as PcCpr (SEQ ID NO: 3) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the 4-hydroxytryptamine kinase (EC 2.7.1.222) is preferably a heterologous 4-hydroxytryptamine kinase such as PcPsiK (SEQ ID NO: 4) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the norbaeocystin N-methyl transferase/psilocybin synthase (EC 2.1.1.345) is a heterologous psilocybin synthase such as PcPsiM (SEQ ID NO: 5) or a functional variant thereof having at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the serotonin N-acetyltransferase (EC 2.3.1.87) is a heterologous serotonin N-acetyltransferase such as BtAANAT (SEQ ID NO: 11) or a functional variant thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the first polynucleotide comprises or consists of SEQ ID NO: 6 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the second polynucleotide comprises or consists of SEQ ID NO: 7 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the third polynucleotide comprises or consists of SEQ ID NO: 8 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the fourth polynucleotide comprises or consists of SEQ ID NO: 9 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the fifth polynucleotide comprises or consists of SEQ ID NO: 10 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the sixth polynucleotide comprises or consists of SEQ ID NO: 30 or a homologue thereof having at least 80% homology, such as at least 85% homology, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In a preferred embodiment, the present invention provides a nucleic acid construct for modifying a yeast cell, said construct comprising:
a first polynucleotide comprising or consisting of SEQ ID NO: 6 or a homologue thereof having at least 80% homology, such as at least 85%, such as at least 90%, such as at least 95% homology thereto;
a second polynucleotide comprising or consisting of SEQ ID NO: 7 or a homologue thereof having at least 80% homology, such as at least 85%, such as at least 90%, such as at least 95% homology thereto;
a third polynucleotide comprising or consisting of SEQ ID NO: 8 or a homologue thereof having at least 80% homology, such as at least 85%, such as at least 90%, such as at least 95% homology thereto; and
optionally a sixth polynucleotide comprising or consisting of SEQ ID NO: 30 or a homologue thereof having at least 80% homology, such as at least 85%, such as at least 90%, such as at least 95% homology thereto.

In a preferred embodiment, the present invention provides a nucleic acid construct for modifying a yeast cell, said construct comprising:
a first polynucleotide comprising or consisting of SEQ ID NO: 6 or a homologue thereof having at least 80% homology, such as at least 85%, such as at least 90%, such as at least 95% homology thereto;
a second polynucleotide comprising or consisting of SEQ ID NO: 7 or a homologue thereof having at least 80% homology, such as at least 85%, such as at least 90%, such as at least 95% homology thereto;
a third polynucleotide comprising or consisting of SEQ ID NO: 8 or a homologue thereof having at least 80% homology, such as at least 85%, such as at least 90%, such as at least 95% homology thereto;
a fourth polynucleotide comprising or consisting of SEQ ID NO: 9 or a homologue thereof having at least 80% homology, such as at least 85%, such as at least 90%, such as at least 95% homology thereto; and
optionally a sixth polynucleotide comprising or consisting of SEQ ID NO: 30 or a homologue thereof having at least 80% homology, such as at least 85%, such as at least 90%, such as at least 95% homology thereto.

In a preferred embodiment, the present invention provides a nucleic acid construct for modifying a yeast cell, said construct comprising:
a first polynucleotide comprising or consisting of SEQ ID NO: 6 or a homologue thereof having at least 80% homology, such as at least 85%, such as at least 90%, such as at least 95% homology thereto;
a second polynucleotide comprising or consisting of SEQ ID NO: 7 or a homologue thereof having at least 80% homology, such as at least 85%, such as at least 90%, such as at least 95% homology thereto;
a third polynucleotide comprising or consisting of SEQ ID NO: 8 or a homologue thereof having at least 80% homology, such as at least 85%, such as at least 90%, such as at least 95% homology thereto;
a fourth polynucleotide comprising or consisting of SEQ ID NO: 9 or a homologue thereof having at least 80% homology, such as at least 85%, such as at least 90%, such as at least 95% homology thereto;
a fifth polynucleotide comprising or consisting of SEQ ID NO: 10 or a homologue thereof having at least 80% homology, such as at least 85%, such as at least 90%, such as at least 95% homology thereto; and
optionally a sixth polynucleotide comprising or consisting of SEQ ID NO: 30 or a homologue thereof having at least 80% homology, such as at least 85%, such as at least 90%, such as at least 95% homology thereto.

In some embodiments, one or more of the first, second, third, fourth, fifth or sixth polynucleotide(s) is/are codon-optimised for said yeast cell.

In some embodiments, each of the nucleic acids encoding each of the present activities, i.e. a tryptophan decarboxylase, a tryptamine monooxygenase, a cytochrome P450 reductase, a norbaeocystin N-methyl transferase/psilocybin synthase and a serotonin N-acetyltransferase may be designed to be integrated within the genome of the yeast cell or they may be within one or more vectors comprised within the yeast cell.

In some embodiments, one or more of the nucleic acids encoding each of the present activities may be integrated in the genome of said yeast cell. Methods for integrating a nucleic acid are well known in the art. Thus in some embodiments the activity of interest is encoded by introduction of a heterologous nucleic acid in the yeast cell. The heterologous nucleic acid encoding said activity may be codon-optimised, or may comprise features that can help improve the activity. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid e.g. in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the activities of interest may be present in high copy number.

In some embodiments, the nucleic acid construct further comprises or consists of one or more vectors, such as an integrative vector or a replicative vector. In some embodiments, the vector is a high copy replicative vector.

Each of the nucleic acid sequences comprised within the present nucleic acid constructs may be present in multiple copies. In some embodiments, at least one of the nucleic acid sequences is present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies. In some embodiments, all of the nucleic acid sequences are present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies.

The nucleic acid constructs may, in addition to the first, second, third, fourth, fifth and sixth polynucleotides described above, also comprise additional polynucleotides useful for introducing additional modifications in the yeast cell, to obtain cells as described in “Other modifications”. Designing such additional polynucleotides can be performed as is known in the art.

The nucleic acid constructs may be a PCR product or a synthetic DNA molecule.

Kit of Parts

Also provided herein is a kit of parts comprising a yeast cell, and/or a nucleic acid construct as described herein, and instructions for use.

In some embodiments, the kit comprises a yeast cell that can be used in the methods described herein. In other embodiments, the kit comprises a nucleic acid construct that can be used to engineer a yeast cell useful for the methods described herein. In some embodiments, the kit comprises a yeast cell and a nucleic acid construct as described herein.

In some embodiments, the kit comprises a yeast cell capable of producing 4-hydroxytryptamine, wherein the yeast cell expresses a tryptophan decarboxylase, a tryptamine monooxygenase and a cytochrome P450 reductase. In some embodiments, the kit comprises a yeast cell capable of producing norbaeocystin, wherein the yeast cell expresses a tryptophan decarboxylase, a tryptamine monooxygenase, a cytochrome P450 reductase and a 4-hydroxytryptamine kinase. In some embodiments, the kit comprises a yeast cell capable of producing baeocystin and optionally norpsilocin, wherein the yeast cell expresses a tryptophan decarboxylase, a tryptamine monooxygenase, a cytochrome P450 reductase, a 4-hydroxytryptamine kinase and a psilocybin synthase. In some embodiments, the kit comprises a yeast cell capable of producing psilocybin and optionally psilocin, wherein the yeast cell expresses a tryptophan decarboxylase, a tryptamine monooxygenase, a cytochrome P450 reductase, a 4-hydroxytryptamine kinase and a psilocybin synthase. In some embodiments, the kit comprises a yeast cell capable of producing aeruginascin and optionally dephosphorylated aeruginascin, wherein the yeast cell expresses a tryptophan decarboxylase, a tryptamine monooxygenase, a cytochrome P450 reductase, a 4-hydroxytryptamine kinase and a psilocybin synthase. In some embodiments, the kit comprises a yeast cell capable of producing N-acetyl-4-hydroxytryptamine, wherein the yeast cell expresses a tryptophan decarboxylase, a tryptamine monooxygenase, a cytochrome P450 reductase, a serotonin N-acetyltransferase and optionally a 4-hydroxytryptamine kinase and a psilocybin synthase. The yeast cell may be further modified as detailed in “Other modifications”.

In some embodiments, the kit comprises a nucleic construct comprising a first polynucleotide encoding a tryptophan decarboxylase, a second polynucleotide encoding a tryptamine monooxygenase and a third polynucleotide encoding a cytochrome P450 reductase. In some embodiments, the kit comprises a nucleic construct comprising a first polynucleotide encoding a tryptophan decarboxylase, a second polynucleotide encoding a tryptamine monooxygenase, a third polynucleotide encoding a cytochrome P450 reductase and a fourth polynucleotide encoding a 4-hydroxytryptamine kinase. In some embodiments, the kit comprises a nucleic construct comprising a first polynucleotide encoding a tryptophan decarboxylase, a second polynucleotide encoding a tryptamine monooxygenase, a third polynucleotide encoding a cytochrome P450 reductase, a fourth polynucleotide encoding a 4-hydroxytryptamine kinase and a fifth polynucleotide encoding a psilocybin synthase. Additionally, any of the previously cited may also comprise a sixth polynucleotide encoding a serotonin N-acetyltransferase.

In some embodiments, the kit comprises the nucleic acid construct as described herein and the yeast cell to be modified. In some embodiments, the yeast cell to be modified is a Saccharomyces cerevisiae cell or a Yarrowia lipolytica cell.

In some embodiments, the kit comprises the yeast cell and a nucleic acid construct as described herein.

Cell Capable of Producing Halogenated Tryptophans and Derivatives Thereof

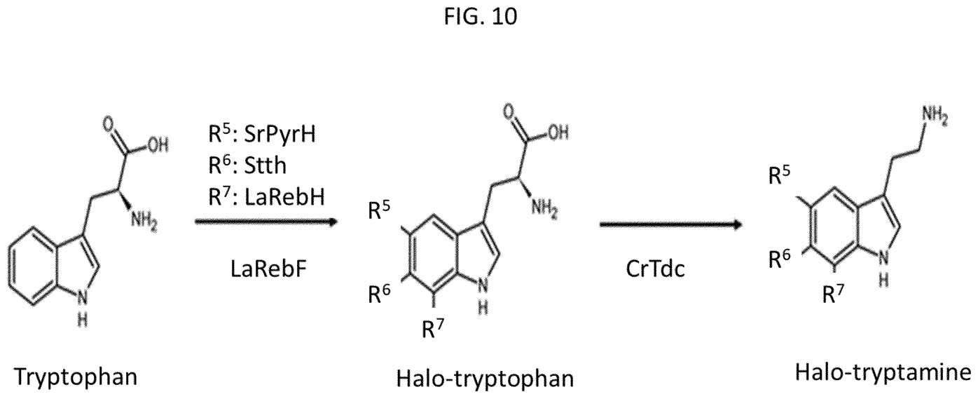

The present disclosure relates to a cell capable of producing a halogenated tryptophan and optionally derivatives thereof, as outlined in FIG. 10. The inventors have found that heterologous expression of a tryptophan-2-halogenase, a tryptophan-5-halogenase, a tryptophan-6-halogenase or a tryptophan-7-halogenase, and optionally a flavin reductase in a cell results in the production of halogenated tryptophan. Other derivatives can also be obtained by expressing additional enzymes in the cell, as described in detail herein. The cell can be engineered as described herein; the nature of the desired product will dictate which enzymes should be introduced in the cell, for example this can be done as illustrated in FIG. 10. The cell is preferably non-naturally occurring.

In some aspects, the present invention provides a cell capable of producing a halogenated tryptophan, wherein the halogenated tryptophan is a tryptophan substituted with one, two or three halogen atoms, and optionally derivatives thereof, in the presence of a halogen or derivatives thereof, said cell expressing at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally expresses a flavin reductase (EC 1.5.1.30), preferably a heterologous flavin reductase, such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
   whereby the cell is capable of converting tryptophan into a halogenated tryptophan and optionally derivatives thereof, a dihalogenated tryptophan and optionally derivatives thereof, or a trihalogenated tryptophan and optionally derivatives thereof,

preferably wherein the cell is a microorganism or a plant cell.

In some embodiments, the cell is a eukaryotic cell or a bacteria or the plant cell is a microalgae cell.

The eukaryotic cell may be selected from the group consisting of the genus of Aspergillus, e.g. A. niger, A. awamori, A. oryzae, and A. nidulans.

The bacteria may be selected from the group consisting of a species belonging to the genus Bacillus, such as B. subtilis, a species belonging to the genus Escherichia, such as E. coli, a species belonging to the genus Lactobacillus, such as L. casei, a species belonging to the genus Lactococcus, such as L. lactis, a species belonging to the genus Corynebacterium, such as C. glutamicum, a species belonging to the genus

Acetobacter, a species belonging to the genus Acinetobacter, a species belonging to the genus Pseudomonas, such as P. putida, and a species belonging to the genus Streptomyces, such as S. coelicolor.

The plant may be selected from the group consisting of a species belonging to the genus Arabidopsis, such as A. thaliana, a species belonging to the genus Zea, such as Z. mays, a species belonging to the genus Medicago, such as M. truncatula, a species belonging to the genus Nicotiana, such as N. tabacum, and a species belonging to the genus Glycine, such as G. Max.

In some embodiments, the cell is a yeast cell. The yeast cell may belong to the genus of Saccharomyces, such as S. cerevisiae, S. kluyveri, S. bayanus, S. exiguus, S. sevazzi, S. uvarum, S. boulardii, a yeast belonging to the genus Kluyveromyces, such as K. lactis, K. marxianus var. marxianus, K. thermotolerans, or belong to the genus Candida, such as C. utilis, C. tropicalis, C. albicans, C. lipolytica, C. versatilis, or belong to the genus Pichia, such as P. stipidis, P. pastoris, P. sorbitophila, or other yeast genera such as Cryptococcus, such as C. aerius, Debaromyces, such as D. hansenii, Hansenula, Pichia, such as P. pastoris, Yarrowia, such as Y. lipolytica, Zygosaccharomyces, such as Z. bailii, Torulaspora, such as T. delbrueckii, Schizosaccharomyces, such as S. pombe, Brettanomyces, such as B. bruxellensis, Penicillium, Rhizopus, Fusarium, Fusidium, Gibberella, Mucor, Mortierella, and Trichoderma. In preferred embodiments, the yeast cell is a Saccharomyces cerevisiae cell or a Yarrowia lipolytica cell.

Production of Halogenated Tryptophans

The cell of the present disclosure can produce halogenated tryptophans. This requires that the cell expresses a tryptophan halogenase and optionally a flavin reductase, whereby the cell is capable of converting tryptophan to a halogenated tryptophan. Preferably the cell is a yeast cell as described herein above.

Tryptophan Halogenase

Depending on which type of halogenation is desired, different tryptophan halogenases can be used. If a 2-halogenated compound is desired, the tryptophan halogenase is a tryptophan-2-halogenase. If a 5-halogenated compound is desired, the tryptophan halogenase is a tryptophan-5-halogenase. If a 6-halogenated compound is desired, the tryptophan halogenase is a tryptophan-6-halogenase. If a 7-halogenated compound is desired, the tryptophan halogenase is a tryptophan-7-halogenase. It is also possible to express several halogenases to obtain dihalogenated or trihalogenated compounds. By way of example, expression of a tryptophan-5-halogenase and a tryptophan-6-halogenase can be employed to obtain 5,6-dihalogenated compounds.

In some embodiments, the tryptophan halogenase is a tryptophan-2-halogenase such as a heterologous tryptophan-2-halogenase (EC 1.14.14). In some embodiments, the tryptophan-2-halogenase is CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-2-halogenase is capable of converting tryptophan to a 2-halogenated tryptophan.

In some embodiments, the tryptophan halogenase is a tryptophan-5-halogenase such as a heterologous tryptophan-5-halogenase (EC 1.14.19.58). In some embodiments, the tryptophan-5-halogenase is SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-5-halogenase is capable of converting tryptophan to a 5-halogenated tryptophan.

In some embodiments, the tryptophan halogenase is a tryptophan-6-halogenase such as a heterologous tryptophan-6-halogenase (EC 1.14.19.59). In some embodiments, the tryptophan-6-halogenase is SttH (SEQ ID NO: 33) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-6-halogenase is capable of converting tryptophan to a 6-halogenated tryptophan.

In some embodiments, the tryptophan halogenase is a tryptophan-6-halogenase such as a heterologous tryptophan-6-halogenase (EC 1.14.19.59). In some embodiments, the tryptophan-6-halogenase is SaThaI (SEQ ID NO: 51) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-6-halogenase is capable of converting tryptophan to a 6-halogenated tryptophan.

In some embodiments, the tryptophan halogenase is a tryptophan-6-halogenase such as a heterologous tryptophan-6-halogenase (EC 1.14.19.59). In some embodiments, the tryptophan-6-halogenase is KtzR (SEQ ID NO: 54) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-6-halogenase is capable of converting tryptophan to a 6-halogenated tryptophan.

In some embodiments, the tryptophan halogenase is a tryptophan-7-halogenase such as a heterologous tryptophan-7-halogenase (EC 1.14.19.9). In some embodiments, the tryptophan-7-halogenase is LaRebH (SEQ ID NO: 34) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-7-halogenase is capable of converting tryptophan to a 7-halogenated tryptophan.

In some embodiments, the tryptophan halogenase is a tryptophan-7-halogenase such as a heterologous tryptophan-7-halogenase (EC 1.14.19.9). In some embodiments, the tryptophan-7-halogenase is PfPrnA (SEQ ID NO: 50) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-7-halogenase is capable of converting tryptophan to a 7-halogenated tryptophan.

In some embodiments, the tryptophan halogenase is a tryptophan-7-halogenase such as a heterologous tryptophan-7-halogenase (EC 1.14.19.9). In some embodiments, the tryptophan-7-halogenase is KtzQ (SEQ ID NO: 53) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan-7-halogenase is capable of converting tryptophan to a 7-halogenated tryptophan.

In some embodiments, the tryptophan halogenase is DdChlA (SEQ ID NO: 52) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan halogenase is capable of converting tryptophan to a halogenated tryptophan.

In some embodiments, the halogen is selected from the group consisting of fluorine, bromine, iodine and chlorine.

In some embodiments, the cell expresses two tryptophan halogenases independently selected from the group consisting of a heterologous tryptophan-2-halogenase, a heterologous tryptophan-5-halogenase, a heterologous tryptophan-6-halogenase and a heterologous tryptophan-7-halogenase, whereby the cell is capable of converting tryptophan to a 2,5-dihalogenated, a 2,6-dihalogenated, a 2,7-dihalogenated, a 5,6-dihalogenated, a 5,7-dihalogenated or a 6,7-dihalogenated tryptophan.

For example, the cell expresses a tryptophan-2-halogenase as described above, for example CcCmdE or a functional variant thereof; and
a tryptophan-5-halogenase as described above, for example SrPyrH or a functional variant thereof;
a tryptophan-6-halogenase as described above, for example SttH, SaThaI or KtzR, or a functional variant thereof;
a tryptophan-7-halogenase as described above, for example LaRebH, PfPrnA or KtzQ, or a functional variant thereof; or
DdChlA or a functional variant thereof.

In some embodiments the cell expresses a tryptophan-5-halogenase as described above, for example SrPyrH or a functional variant thereof, and:
a tryptophan-2-halogenase as described above, for example CcCmdE or a functional variant thereof; and
a tryptophan-6-halogenase as described above, for example SttH, SaThaI or KtzR, or a functional variant thereof;
a tryptophan-7-halogenase as described above, for example LaRebH, PfPrnA or KtzQ, or a functional variant thereof; or
DdChlA or a functional variant thereof.

In some embodiments the cell expresses a tryptophan-6-halogenase as described above, for example SttH, SaThaI or KtzR, or a functional variant thereof, and:
a tryptophan-2-halogenase as described above, for example CcCmdE or a functional variant thereof; and
tryptophan-5-halogenase as described above, for example SrPyrH or a functional variant thereof;
a tryptophan-7-halogenase as described above, for example LaRebH, PfPrnA or KtzQ, or a functional variant thereof; or
DdChlA or a functional variant thereof.

In some embodiments the cell expresses a tryptophan-7-halogenase as described above, for example LaRebH, PfPrnA or KtzQ, or a functional variant thereof, and:
a tryptophan-2-halogenase as described above, for example CcCmdE or a functional variant thereof; and
tryptophan-5-halogenase as described above, for example SrPyrH or a functional variant thereof;
a tryptophan-6-halogenase as described above, for example SttH, SaThaI or KtzR, or a functional variant thereof; or
DdChlA or a functional variant thereof.

In some embodiments the cell expresses DdChlA or a functional variant thereof, and:
a tryptophan-2-halogenase as described above, for example CcCmdE or a functional variant thereof; and
tryptophan-5-halogenase as described above, for example SrPyrH or a functional variant thereof;
a tryptophan-6-halogenase as described above, for example SttH, SaThaI or KtzR, or a functional variant thereof; or
a tryptophan-7-halogenase as described above, for example LaRebH, PfPrnA or KtzQ, or a functional variant thereof.

In some embodiments, the cell expresses three tryptophan halogenases independently selected from the group consisting of a heterologous tryptophan-2-halogenase, a heterologous tryptophan-5-halogenase, a heterologous tryptophan-6-halogenase and a heterologous tryptophan-7-halogenase, whereby the cell is capable of converting tryptophan to a 2,5,6-trihalogenated, a 2,5,7-trihalogenated, a 2,6,7-trihalogenated or a 5,6,7-trihalogenated tryptophan.

For example, the cell expresses a tryptophan-2-halogenase as described above, for example CcCmdE or a functional variant thereof; and a tryptophan-5-halogenase as described above, for example SrPyrH or a functional variant thereof; and
a tryptophan-6-halogenase as described above, for example SttH, SaThaI or KtzR, or a functional variant thereof;
a tryptophan-7-halogenase as described above, for example LaRebH, PfPrnA or KtzQ, or a functional variant thereof; or
DdChlA or a functional variant thereof.

In some embodiments, the cell expresses a tryptophan-2-halogenase as described above, for example CcCmdE or a functional variant thereof; and a tryptophan-6-halogenase as described above, for example SttH, SaThaI or KtzR; and
a tryptophan-7-halogenase as described above, for example LaRebH, PfPrnA or KtzQ, or a functional variant thereof; or
DdChlA or a functional variant thereof.

In some embodiments, the cell expresses a tryptophan-2-halogenase as described above, for example CcCmdE or a functional variant thereof; and a tryptophan-7-halogenase as described above, for example LaRebH, PfPrnA or KtzQ; and DdChlA or a functional variant thereof.

In some embodiments, the cell expresses a tryptophan-5-halogenase as described above, for example SrPyrH or a functional variant thereof; and a tryptophan-6-halogenase as described above, for example SttH, SaThaI or KtzR or a functional variant thereof; and
DdChlA, or a functional variant thereof; or
a tryptophan-7-halogenase as described above, for example LaRebH, PfPrnA or KtzQ or a functional variant thereof.

In some embodiments, the cell expresses a tryptophan-5-halogenase as described above, for example SrPyrH or a functional variant thereof; and a tryptophan-7-halogenase as described above, for example LaRebH, PfPrnA or KtzQ or a functional variant thereof; and DdChIA.

In some embodiments, the cell expresses a tryptophan-6-halogenase as described above, for example SttH, SaThaI or KtzR or a functional variant thereof; DdChlA or a functional variant thereof; and a tryptophan-7-halogenase as described above, for example LaRebH, PfPrnA or KtzQ or a functional variant thereof.

Flavin Reductase

In order to increase the titer of halogenated tryptophan produced by the present cells, it may be helpful to express in the cell a flavin reductase in addition to one or more tryptophan halogenases as described herein above.

In some embodiments, the flavin reductase (EC 1.5.1.30) is a heterologous flavin reductase. In some embodiments, the flavin reductase is LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the flavin reductase is capable of reducing FAD to FADH₂.

In some embodiments, the cell expresses CcCmdE, SrPyrH, SttH and/or LaRebH, and optionally LaRebF as set forth in SEQ ID NO: 48, SEQ ID NO: 32, SEQ ID NO: 33, SEQ ID NO: 34 and SEQ ID NO: 35, respectively, or functional variants thereof having at least 80% homology thereto, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Titers

In some embodiments, the cell is capable of producing a 2-halogenated, a 5-halogenated, a 6-halogenated, or a 7-halogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5-dihalogenated, a 2,6-dihalogenated, a 2,7-dihalogenated, a 5,6-dihalogenated, a 5,7-dihalogenated or a 6,7-dihalogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5,6-trihalogenated, a 2,5,7-trihalogenated, a 2,6,7-trihalogenated or a 5,6,7-trihalogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

The cell may be further engineered to allow production of halogenated tryptophan derivatives, as described herein below in detail. Such derivatives include halogenated tryptamine, halogenated N-methyltryptamine, halogenated N,N-dimethyltryptamine and halogenated N,N,N-trimethyltryptamine. FIG. 10 provides an overview of some of the products that can be obtained using the present cells, depending on which enzymes are expressed.

Methods for determining the titer are known in the art, for example by measuring the peak area from LC-MS analysis and comparing to the peak area of an authentic analytical standard of known concentration.

Production of Halogenated Tryptamines

The cell may be further engineered to produce halogenated tryptamines from halogenated tryptophans. This can be achieved by introducing a tryptophan decarboxylase capable of converting the halogenated tryptophan to a corresponding halogenated tryptamine. Preferably the cell is a yeast cell as described herein above.

Tryptophan Decarboxylase

Expression of a tryptophan decarboxylase in the above cells capable of producing halogenated tryptamines results in cells capable of producing the corresponding halogenated tryptamines.

In some embodiments, the cell further expresses a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting the halogenated tryptophan to a corresponding halogenated tryptamine, whereby the cell is capable converting the halogenated tryptophan to a corresponding halogenated tryptamine. The cell can thus produce the corresponding halogenated tryptamine.

In some embodiments, the tryptophan decarboxylase (EC 4.1.1.105) is a heterologous tryptophan decarboxylase. In some embodiments, the tryptophan decarboxylase is CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the cell is capable of producing a halogenated tryptamine, wherein the halogenated tryptamine is a tryptamine substituted with one, two or three halogen atoms, and said cell expresses at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally expresses a flavin reductase (EC 1.5.1.30), preferably a heterologous flavin reductase, such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
   wherein the tryptophan halogenase(s) catalyze(s) the conversion of tryptophan to a halogenated tryptophan, whereby the cell is capable of converting tryptophan into a halogenated tryptophan,

and said cell also expresses a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase catalyzes the conversion of the halogenated tryptophan to a corresponding halogenated tryptamine, whereby the cell is capable of converting the halogenated tryptophan into a corresponding halogenated tryptamine,
preferably wherein the cell is a microorganism or a plant cell.

2-halo-tryptophan can be used to produce 2-halo-tryptamine. 5-halo-tryptophan can be used to produce 5-halo-tryptamine. 6-halo-tryptophan can be used to produce 6-halo-tryptamine. 7-halo-tryptophan can be used to produce 7-halo-tryptamine.

2,5-dihalo-tryptophan can be used to produce 2,5-dihalo-tryptamine. 2,6-dihalo-tryptophan can be used to produce 2,6-dihalo-tryptamine. 2,7-dihalo-tryptophan can be used to produce 2,7-dihalo-tryptamine. 5,6-dihalo-tryptophan can be used to produce 5,6-dihalo-tryptamine. 5,7-dihalo-tryptophan can be used to produce 5,7-dihalo-tryptamine. 6,7-dihalo-tryptophan can be used to produce 6,7-dihalo-tryptamine.

2,5,6-trihalo-tryptophan can be used to produce 2,5,6-trihalo-tryptamine. 2,5,7-trihalo-tryptophan can be used to produce 2,5,7-trihalo-tryptamine. 2,6,7-trihalo-tryptophan can be used to produce 2,6,7-trihalo-tryptamine. 5,6,7-trihalo-tryptophan can be used to produce 5,6,7-trihalo-tryptamine.

In some embodiments, the cell expresses CcCmdE, SrPyrH, SttH and/or LaRebH, and optionally LaRebF as set forth in SEQ ID NO: 48, SEQ ID NO: 32, SEQ ID NO: 33, SEQ ID NO: 34 and SEQ ID NO: 35, respectively, or functional variants thereof having at least 80% homology thereto, and further expresses CrTDC as set forth in SEQ ID NO: 1 or a functional variant thereof having at least 80% homology thereto.

Titers

In some embodiments, the cell is capable of producing a 2-halogenated tryptamine, a 5-halogenated tryptamine, a 6-halogenated tryptamine, and/or a 7-halogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5-dihalogenated tryptamine, a 2,6-dihalogenated tryptamine, a 2,7-dihalogenated tryptamine, a 5,6-dihalogenated tryptamine, a 5,7-dihalogenated tryptamine, and/or a 6,7-dihalogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5,6-trihalogenated tryptamine, a 2,5,7-trihalogenated tryptamine, a 2,6,7-trihalogenated tryptamine and/or a 5,6,7-trihalogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Methods for determining the titer are known in the art, for example by measuring the peak area from LC-MS analysis and comparing to the peak area of an authentic analytical standard of known concentration.

Production of Halogenated N-Methylated, N,N-Dimethylated and N,N,N-Trimethylated Tryptamines

The cell may be further engineered to produce halogenated N-methylated, halogenated N,N-dimethylated and/or halogenated N,N,N-trimethylated tryptamine from halogenated tryptamine. This can be achieved by introducing an indole N-methyltransferase capable of converting the halogenated tryptamine produced by the cell to the corresponding halogenated N-methylated, halogenated N,N-dimethylated and/or halogenated N,N,N-trimethylated tryptamine. The cell may be any of the cells described herein. Preferably the cell is a yeast cell as described herein above.

Indole N-Methyltransferase

In some embodiments, the cell is any of the cells described herein above which is capable of producing a halogenated tryptamine. In order to produce halogenated, methylated tryptamine, the cell in some embodiments further expresses an indole N-methyltransferase (EC 2.1.1.49), preferably a heterologous indole N-methyltransferase, such as OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase is capable of converting the halogenated tryptamine to a corresponding halogenated N-methylated, halogenated N,N-dimethylated and/or halogenated N,N,N-trimethylated tryptamine, whereby the cell is capable of converting halogenated tryptamine to halogenated N-methylated, halogenated N,N-dimethylated and/or halogenated N,N,N-trimethylated tryptamine. The cell can thus produce halogenated N-methylated, halogenated N,N-dimethylated and/or halogenated N,N,N-trimethylated tryptamine.

In some embodiments, the indole N-methyltransferase (EC 2.1.1.49) is a heterologous indole N-methyltransferase. In some embodiments, the indole N-methyltransferase is OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the cell is capable of producing halogenated N-methylated, halogenated N,N-dimethylated and/or halogenated N,N,N-trimethylated tryptamine, wherein the halogenated N-methyltryptamine, the halogenated N,N-dimethyltryptamine or the halogenated N,N,N-trimethyltryptamine is N-methyltryptamine, N,N-dimethyltryptamine or N,N,N-trimethyltryptamine, respectively, substituted with one, two or three halogen atoms, and said cell expresses at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally expresses a flavin reductase (EC 1.5.1.30), preferably a heterologous flavin reductase, such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
   wherein the tryptophan halogenase(s) catalyze(s) the conversion of tryptophan to a halogenated tryptophan, whereby the cell is capable of converting tryptophan into a halogenated tryptophan,

also expresses a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase catalyzes the conversion of the halogenated tryptophan to the corresponding halogenated tryptamine, whereby the cell is capable of converting the halogenated tryptophan into the corresponding halogenated tryptamine,
and said cell also expresses an indole N-methyltransferase (EC 2.1.1.49), preferably a heterologous indole N-methyltransferase, such as OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase catalyzes the conversion of the halogenated tryptamine to the corresponding halogenated N-methylated, halogenated N,N-dimethylated and/or halogenated N,N,N-trimethylated tryptamine, whereby the cell is capable of converting the halogenated tryptamine into the corresponding halogenated N-methylated, halogenated N,N-dimethylated and/or halogenated N,N,N-trimethylated tryptamine,
preferably wherein the cell is a microorganism or a plant cell.

2-halo-tryptamine can be used to produce 2-halogenated N-methylated, N,N-dimethylated and N,N,N-trimethylated tryptamine. 5-halo-tryptamine can be used to produce 5-halogenated N-methylated, N,N-dimethylated and N,N,N-trimethylated tryptamine. 6-halo-tryptamine can be used to produce 6-halogenated N-methylated, N,N-dimethylated and N,N,N-trimethylated tryptamine. 7-halo-tryptamine can be used to produce 7-halogenated N-methylated, N,N-dimethylated and N,N,N-trimethylated tryptamine.

2,5-dihalo-tryptamine can be used to produce 2,5-dihalogenated N-methylated, N,N-dimethylated and N,N,N-trimethylated tryptamine. 2,6-dihalo-tryptamine can be used to produce 2,6-dihalogenated N-methylated, N,N-dimethylated and N,N,N-trimethylated tryptamine. 2,7-dihalo-tryptamine can be used to produce 2,7-dihalogenated N-methylated, N,N-dimethylated and N,N,N-trimethylated tryptamine. 5,6-dihalo-tryptamine can be used to produce 5,6-dihalogenated N-methylated, N,N-dimethylated and N,N,N-trimethylated tryptamine. 5,7-dihalo-tryptamine can be used to produce 5,7-dihalogenated N-methylated, N,N-dimethylated and N,N,N-trimethylated tryptamine. 6,7-dihalo-tryptamine can be used to produce 6,7-dihalogenated N-methylated, N,N-dimethylated and N,N,N-trimethylated tryptamine.

2,5,6-trihalo-tryptamine can be used to produce 2,5,6-trihalogenated N-methylated, N,N-dimethylated and N,N,N-trimethylated tryptamine. 2,5,7-trihalo-tryptamine can be used to produce 2,5,7-trihalogenated N-methylated, N,N-dimethylated and N,N,N-trimethylated tryptamine. 2,6,7-trihalo-tryptamine can be used to produce 2,6,7-trihalogenated N-methylated, N,N-dimethylated and N,N,N-trimethylated tryptamine. 5,6,7-trihalo-tryptamine can be used to produce 5,6,7-trihalogenated N-methylated, N,N-dimethylated and N,N,N-trimethylated tryptamine.

halogenated N-methylated, halogenated N,N-dimethylated and/or halogenated N,N,N-trimethylated tryptamine

In some embodiments, the cell expresses CcCmdE, SrPyrH, SttH and/or LaRebH, as well as CrTDC and optionally LaRebF as set forth in SEQ ID NO: 48, SEQ ID NO: 32, SEQ ID NO: 33, SEQ ID NO: 34, SEQ ID NO: 1 and SEQ ID NO: 35, respectively, or functional variants thereof having at least 80% homology thereto, and further expresses OcINMT as set forth in SEQ ID NO: 36 or a functional variant thereof having at least 80% homology thereto.

Titers

In some embodiments, the cell is capable of producing a 2-halogenated N-methyltryptamine, a 2-halogenated N,N-dimethyltryptamine and/or a 2-halogenated N,N,N-trimethyltryptamine, a 5-halogenated N-methyltryptamine, a 5-halogenated N,N-dimethyltryptamine and/or a 5-halogenated N,N,N-trimethyltryptamine, a 6-halogenated N-methyltryptamine, a 6-halogenated N,N-dimethyltryptamine and/or a 6-halogenated N,N,N-trimethyltryptamine, and/or a 7-halogenated N-methyltryptamine, a 7-halogenated N,N-dimethyltryptamine and/or a 7-halogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5-dihalogenated N-methyltryptamine, a 2,5-dihalogenated N,N-dimethyltryptamine and/or a 2,5-dihalogenated N,N,N-trimethyltryptamine, a 2,6-dihalogenated N-methyltryptamine, a 2,6-dihalogenated N,N-dimethyltryptamine and/or a 2,6-dihalogenated N,N,N-trimethyltryptamine, a 2,7-dihalogenated N-methyltryptamine, a 2,7-dihalogenated N,N-dimethyltryptamine and/or a 2,7-dihalogenated N,N,N-trimethyltryptamine, a 5,6-dihalogenated N-methyltryptamine, a 5,6-dihalogenated N,N-dimethyltryptamine and/or a 5,6-dihalogenated N,N,N-trimethyltryptamine, a 5,7-dihalogenated N-methyltryptamine, a 5,7-dihalogenated N,N-a dimethyltryptamine and/or a 5,7-dihalogenated N,N,N-trimethyltryptamine, and/or a 6,7-dihalogenated N-methyltryptamine, a 6,7-dihalogenated N,N-dimethyltryptamine and/or a 6,7-dihalogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell is capable of producing a 2,5,6-trihalogenated N-methyltryptamine, a 2,5,6-trihalogenated N,N-dimethyltryptamine and/or a 2,5,6-trihalogenated N,N,N-trimethyltryptamine, a 2,5,7-trihalogenated N-methyltryptamine, a 2,5,7-trihalogenated N,N-dimethyltryptamine and/or a 2,5,7-trihalogenated N,N,N-trimethyltryptamine, a 2,6,7-trihalogenated N-methyltryptamine, a 2,6,7-trihalogenated N,N-dimethyltryptamine and/or a 2,6,7-trihalogenated N,N,N-trimethyltryptamine, a 5,6,7-trihalogenated N-methyltryptamine, a 5,6,7-trihalogenated N,N-dimethyltryptamine and/or a 5,6,7-trihalogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the total titer of all halogenated compounds produced by the cell is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more, wherein the total titer is the sum of the titers of the 2-halogenated, 5-halogenated, 6-halogenated, 7-halogenated, 2,5-dihalogenated, 2,6-dihalogenated, 2,7-dihalogenated, 5,6-dihalogenated, 5,7-dihalogenated, 6,7-dihalogenated, 2,5,6-trihalogenated, 2,5,7-trihalogenated, 2,6,7-trihalogenated and 5,6,7-trihalogenated tryptophans, tryptamines, N-methyltryptamines, N,N-dimethyltryptamines and N,N,N-trimethyltryptamines.

Methods for determining the titer are known in the art, for example by measuring the peak area from LC-MS analysis and comparing to the peak area of an authentic analytical standard of known concentration.

Expression of Heterologous Enzymes

In some embodiments, one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is under the control of an inducible promoter.

In some embodiments, one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is codon-optimised for the cell, as is known in the art.

In some embodiments, one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is present in 2 to 30 copies.

In some embodiments, one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is integrated in the genome of the cell.

In some embodiments, one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is expressed from a vector such as a plasmid.

In some embodiments, expression of one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase can be induced or repressed, for instance to obtain transient expression, as is known in the art.

Nucleic acid constructs useful for obtaining yeast cells capable of halogenated tryptophan or derivatives thereof are described in the section “Nucleic acid constructs”.

Other Modifications for Increasing Titers

Because the present pathways require tryptophan as a first substrate, and without being bound by theory, it may be advantageous to modify the cell in such a manner that tryptophan metabolism is directed towards increased tryptophan synthesis, thereby further increasing the titers of the halogenated tryptophans or derivatives thereof. Examples of these modifications can be found in the section “Other modifications”.

Examples of Useful Cells

In this section a number of specific cells representing specific embodiments of cells useful for production of halogenated compounds are listed. In some embodiments, the cell is a yeast cell. Preferably the yeast cell is a Saccharomyces cerevisiae cell or a Yarrowia lipolytica cell.

In a specific embodiment, the cell is capable of producing a chlorinated, a fluorinated, a brominated or a iodinated tryptophan, and expresses:
SrPyrH (SEQ ID NO: 32); and
LaRebF (SEQ ID NO: 35),

Or functional variants thereof having at least 80% homology thereto. Preferably the cell is a yeast cell as described herein above.

In another embodiment, the cell is capable of producing a chlorinated, a fluorinated, a brominated or a iodinated tryptophan, and expresses:
SttH (SEQ ID NO: 33); and
LaRebF (SEQ ID NO: 35),

Or functional variants thereof having at least 80% homology thereto. Preferably the cell is a yeast cell as described herein above.

In another embodiment, the cell is capable of producing a chlorinated, a fluorinated, a brominated or a iodinated tryptophan, and/or a chlorinated, a fluorinated, a brominated or a iodinated tryptamine, and expresses:
SrPyrH (SEQ ID NO: 32);
LaRebF (SEQ ID NO: 35); and
CrTDC (SEQ ID NO: 1),

Or functional variants thereof having at least 80% homology thereto. Preferably the cell is a yeast cell as described herein above.

In another embodiment, the cell is capable of producing a chlorinated, a fluorinated, a brominated or a iodinated tryptophan, and/or a chlorinated, a fluorinated, a brominated or a iodinated tryptamine, and expresses:
SttH (SEQ ID NO: 33);
LaRebF (SEQ ID NO: 35); and
CrTDC (SEQ ID NO: 1),

Or functional variants thereof having at least 80% homology thereto. Preferably the cell is a yeast cell as described herein above.

Other organisms besides yeast may also be useful as production organisms according to the present disclosure. Thus, in some embodiments the production cell is a microorganism or a plant cell. The microorganism may e.g. be a eukaryotic cell, a yeast cell or a bacteria, and the plant cell may e.g. be a microalgae.

Useful eukaryotic cells include eukaryotic cells belonging to the genus of Aspergillus, e.g. A. niger, A. awamori, A. oryzae, and A. nidulans.

Useful bacteria include bacteria belonging to the genus Bacillus, such as B. subtilis, a species belonging to the genus Escherichia, such as E. coli, a species belonging to the genus Lactobacillus, such as L. casei, a species belonging to the genus Lactococcus, such as L. lactis, a species belonging to the genus Corynebacterium, such as C. glutamicum, a species belonging to the genus Acetobacter, a species belonging to the genus Acinetobacter, a species belonging to the genus Pseudomonas, such as P. putida, and a species belonging to the genus Streptomyces, such as S. coelicolor.

Useful yeast cells include a yeast cell belonging to the genus of Saccharomyces, such as S. cerevisiae, S. kluyveri, S. bayanus, S. exiguus, S. sevazzi, S. uvarum, S. boulardii, a yeast belonging to the genus Kluyveromyces, such as K. lactis, K. marxianus var. marxianus, K. thermotolerans, or belong to the genus Candida, such as C. utilis, C. tropicalis, C. albicans, C. lipolytica, C. versatilis, or belong to the genus Pichia, such as P. stipidis, P. pastoris, P. sorbitophila, or other yeast genera such as Cryptococcus, such as C. aerius, Debaromyces, such as D. hansenii, Hansenula, Pichia, such as P. pastoris, Yarrowia, such as Y. lipolytica, Zygosaccharomyces, such as Z. bailii, Torulaspora, such as T. delbrueckii, Schizosaccharomyces, such as S. pombe, Brettanomyces, such as B. bruxellensis, Penicillium, Rhizopus, Fusarium, Fusidium, Gibberella, Mucor, Mortierella, and Trichoderma.

Useful plants include plants belonging to the genus Arabidopsis, such as A. thaliana, a species belonging to the genus Zea, such as Z. mays, a species belonging to the genus Medicago, such as M. truncatula, a species belonging to the genus Nicotiana, such as N. tabacum, and a species belonging to the genus Glycine, such as G. Max.

Methods of Production of Halogenated Tryptophans and Derivatives Thereof

The present disclosure relates to methods for producing halogenated tryptophans and derivatives thereof. The cells and nucleic acid constructs described herein are useful for cell-based production of halogenated tryptophans and derivatives thereof, including halogenated tryptamines, halogenated N-methylated tryptamines, halogenated N,N-dimethylated tryptamines and halogenated N,N,N-trimethylated tryptamines. Throughout the present disclosure, it will be understood that the cells can produce the compounds of interest listed herein when incubated in a cultivation medium under conditions that enable the cell to grow and produce the desired compound. From the description of the production host cells provided herein, and knowing the type of host cell used, the skilled person will not have difficulties in identifying suitable cultivation media and conditions to achieve production. In particular, the cultivation may be performed aerobically or anaerobically, at temperatures and at pH suitable for supporting growth of the cell. The cultivation medium should include the required nutrients, and may be supplemented with precursors as applicable. The time of cultivation will vary depending on which cell is used, but can easily be adapted by the skilled person.

Halogenated Tryptophans

In some aspects, the present invention provides a method of producing a halogenated tryptophan, wherein the halogenated tryptophan is a tryptophan substituted with one, two or three halogen atoms, and optionally derivatives thereof, in a cell, preferably wherein the cell is a microorganism or a plant cell, said method comprising the steps of providing a cell and incubating said cell in the presence of a halogen, wherein the cell expresses at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,

and optionally a flavin reductase, preferably a heterologous flavin reductase (EC: EC 1.5.1.30), such as LaRebF (SEQ ID NO: 35), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the method is for producing a 2-halogenated, a 5-halogenated, a 6-halogenated and/or a 7-halogenated tryptophan.

In some embodiments, the method is for producing a 2,5-dihalogenated, a 2,6-dihalogenated, a 2,7-dihalogenated, a 5,6-dihalogenated, a 5,7-dihalogenated and/or a 6,7-dihalogenated tryptophan.

In some embodiments, the method is for producing a 2,5,6-trihalogenated, a 2,5,7-trihalogenated, a 2,6,7-trihalogenated and/or a 5,6,7-trihalogenated tryptophan.

In some embodiments, the halogen is selected from the group consisting of fluorine, bromine, iodine and chlorine.

Cells useful for producing halogenated tryptophans are described herein, in particular in the section “Production of halogenated tryptophans” herein above.

Herein are also provided methods for producing derivatives of halogenated tryptophans.

Halogenated Tryptamines

In some embodiments, the method is for producing a halogenated tryptamine, wherein the halogenated tryptophan derivative is a halogenated tryptamine. In such embodiments, the cell further expresses a tryptophan decarboxylase (EC 4.1.1.105), preferably a heterologous tryptophan decarboxylase such as CrTDC (SEQ ID NO: 1) or a functional variant thereof having at least homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Cells useful for production of halogenated tryptamines are described herein, in particular in the section “Production of halogenated tryptamines” herein above.

In some embodiments, the method is for producing a halogenated tryptamine, wherein the halogenated tryptamine is a tryptamine substituted with one, two or three halogen atoms, and the cell further expresses a tryptophan decarboxylase (EC 4.1.1.105), preferably a heterologous tryptophan decarboxylase such as CrTDC (SEQ ID NO: 1) or a functional variant thereof having at least homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the method is for producing a halogenated tryptamine in a cell, wherein the halogenated tryptamine is a tryptamine substituted with one, two or three halogen atoms, said method comprising the steps of providing a cell and incubating said cell in a medium, wherein the cell expresses at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally expresses a flavin reductase (EC 1.5.1.30), preferably a heterologous flavin reductase, such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
   wherein the tryptophan halogenase(s) catalyze(s) the conversion of tryptophan to a halogenated tryptophan, whereby the cell is capable of converting tryptophan into a halogenated tryptophan, and said cell also expresses a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase catalyzes the conversion of the halogenated tryptophan to a corresponding halogenated tryptamine, whereby the cell is capable of converting the halogenated tryptophan into a corresponding halogenated tryptamine,
   preferably wherein the cell is a microorganism or a plant cell.

In some embodiments, the method is for producing a 2-halogenated, a 5-halogenated, a 6-halogenated and/or a 7-halogenated tryptamine.

In some embodiments, the method is for producing a 2,5-dihalogenated, a 2,6-dihalogenated, a 2,7-dihalogenated, a 5,6-dihalogenated, a 5,7-dihalogenated and/or a 6,7-dihalogenated tryptamine.

In some embodiments, the method is for producing a 2,5,6-trihalogenated, a 2,5,7-trihalogenated, a 2,6,7-trihalogenated and/or a 5,6,7-trihalogenated tryptamine.

Halogenated N-Methylated, N,N-Dimethylated, and N,N,N-Trimethylated Tryptamine

In some embodiments, the halogenated tryptophan derivative is a halogenated N-methylated tryptamine, a halogenated N,N-dimethylated tryptamine, or a halogenated N,N,N-trimethylated tryptamine. In such embodiments, the cell further expresses an indole N-methyltransferase (EC 2.1.1.49), preferably a heterologous indole N-methyltransferase, such as OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Cells useful for production of halogenated N-methylated tryptamine, halogenated N,N-dimethylated tryptamine, and halogenated N,N,N-trimethylated tryptamine are described herein, in particular in the section “Production of halogenated N-methylated, N,N-dimethylated and N,N,N-trimethylated tryptamines” herein above.

In some embodiments, the method is for producing halogenated N-methylated, halogenated N,N-dimethylated and/or halogenated N,N,N-trimethylated tryptamine, wherein the halogenated N-methyltryptamine, the halogenated N,N-dimethyltryptamine or the halogenated N,N,N-trimethyltryptamine is N-methyltryptamine, N,N-dimethyltryptamine or N,N,N-trimethyltryptamine, respectively, substituted with one, two or three halogen atoms, in a cell, said method comprising the steps of providing a cell and incubating said cell in a medium, wherein the cell expresses at least one of:
a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-5-halogenase (EC 1.14.19.58), preferably a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally expressing a flavin reductase (EC 1.5.1.30), preferably a heterologous flavin reductase, such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
   wherein the tryptophan halogenase(s) catalyze(s) the conversion of tryptophan to a halogenated tryptophan, whereby the cell is capable of converting tryptophan into a halogenated tryptophan,

also expresses a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase catalyzes the conversion of the halogenated tryptophan to the corresponding halogenated tryptamine, whereby the cell is capable of converting the halogenated tryptophan into the corresponding halogenated tryptamine,
and said cell also expresses an indole N-methyltransferase (EC 2.1.1.49), preferably a heterologous indole N-methyltransferase, such as OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase catalyzes the conversion of the halogenated tryptamine to the corresponding halogenated N-methylated, halogenated N,N-dimethylated and/or halogenated N,N,N-trimethylated tryptamine, whereby the cell is capable of converting the halogenated tryptamine into the corresponding halogenated N-methylated, halogenated N,N-dimethylated and/or halogenated N,N,N-trimethylated tryptamine,
preferably wherein the cell is a microorganism or a plant cell.

In some embodiments, the method is for producing a 2-halogenated N-methyltryptamine, a 2-halogenated N,N-dimethyltryptamine and/or a 2-halogenated N, N, N-trimethyltryptamine.

In some embodiments, the method is for producing a 5-halogenated N-methyltryptamine, a 5-halogenated N,N-dimethyltryptamine and/or a 5-halogenated N, N, N-trimethyltryptamine.

In some embodiments, the method is for producing a 6-halogenated N-methyltryptamine, a 6-halogenated N,N-dimethyltryptamine and/or a 6-halogenated N, N, N-trimethyltryptamine.

In some embodiments, the method is for producing a 7-halogenated N-methyltryptamine, a 7-halogenated N,N-dimethyltryptamine and/or a 7-halogenated N, N, N-trimethyltryptamine.

In some embodiments, the method is for producing 2,5-dihalogenated N-methyltryptamine, a 2,5-dihalogenated N,N-dimethyltryptamine and/or a 2,5-dihalogenated N,N,N-trimethyltryptamine.

In some embodiments, the method is for producing a 2,6-dihalogenated N-methyltryptamine, a 2,6-dihalogenated N,N-dimethyltryptamine and/or a 2,6-dihalogenated N, N, N-trimethyltryptamine.

In some embodiments, the method is for producing a 2,7-dihalogenated N-methyltryptamine, a 2,7-dihalogenated N,N-dimethyltryptamine and/or a 2,7-dihalogenated N,N,N-trimethyltryptamine.

In some embodiments, the method is for producing a 5,6-dihalogenated N-methyltryptamine, a 5,6-dihalogenated N,N-dimethyltryptamine and/or a 5,6-dihalogenated N, N, N-trimethyltryptamine.

In some embodiments, the method is for producing a 5,7-dihalogenated N-methyltryptamine, a 5,7-dihalogenated N,N-a dimethyltryptamine and/or a 5,7-dihalogenated N, N, N-trimethyltryptamine.

In some embodiments, the method is for producing a 6,7-dihalogenated N-methyltryptamine, a 6,7-dihalogenated N,N-dimethyltryptamine and/or a 6,7-dihalogenated N,N,N-trimethyltryptamine.

In some embodiments, the method is for producing a 2,5,6-trihalogenated N-methyltryptamine, a 2,5,6-trihalogenated N,N-dimethyltryptamine and/or a 2,5,6-trihalogenated N, N, N-trimethyltryptamine.

In some embodiments, the method is for producing a 2,5,7-trihalogenated N-methyltryptamine, a 2,5,7-trihalogenated N,N-dimethyltryptamine and/or a 2,5,7-trihalogenated N, N, N-trimethyltryptamine.

In some embodiments, the method is for producing a 2,6,7-trihalogenated N-methyltryptamine, a 2,6,7-trihalogenated N,N-dimethyltryptamine and/or a 2,6,7-trihalogenated N, N, N-trimethyltryptamine.

In some embodiments, the method is for producing a 5,6,7-trihalogenated N-methyltryptamine, a 5,6,7-trihalogenated N,N-dimethyltryptamine and/or a 5,6,7-trihalogenated N,N,N-trimethyltryptamine.

Useful Enzymes for Production of Halogenated Tryptophans and Derivatives Thereof

Enzymes useful for the present methods, which can advantageously be introduced in the cell, are described in detail herein above in the section entitled “Cell capable of producing halogenated tryptophans and derivatives thereof”.

In some embodiments, the tryptophan-2-halogenase (EC 1.14.14) is a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan-5-halogenase (EC 1.14.19.58) is a heterologous tryptophan-5-halogenase such as SrPyrH (SEQ ID NO: 32) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan-6-halogenase (EC 1.14.19.59) is a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the tryptophan-6-halogenase is SttH (SEQ ID NO: 33) or a functional variant thereof having at least 80% homology thereto.

In some embodiments, the tryptophan-7-halogenase (EC 1.14.19.9) is a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the tryptophan-7-halogenase is LaRebH (SEQ ID NO: 34) or a functional variant thereof having at least 80% homology thereto.

In some embodiments, the tryptophan halogenase is a heterologous tryptophan halogenase such as DdChlA (SEQ ID NO: 52) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the flavin reductase (EC 1.5.1.30) is a heterologous flavin reductase such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan decarboxylase (EC 4.1.1.105) is a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the indole N-methyltransferase (EC 2.1.1.49) is a heterologous indole N-methyltransferase such a OcINMT (SEQ ID NO: 36) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the medium comprises tryptophan and/or the cell is capable of synthesizing tryptophan.

The cell may further comprise any of the modifications detailed in the section “Other modifications”. For example, in some embodiments, the cell further comprises one or more mutations resulting in increased availability of L-tryptophan.

Recovering the Halogenated Tryptophans and/or Derivatives Thereof

The present methods may comprise a further step of recovering the compounds obtained by the methods disclosed herein. Methods for recovering the products obtained by the present invention are known in the art, for example organic solvent extraction followed by lyophilisation and purification by preparative HPLC or similar column purification techniques. Methods for recovering the products obtained by the present invention are known in the art, for example organic solvent extraction followed by lyophilisation and purification by preparative HPLC or similar column purification techniques. For example, the step of recovering the compound(s) may comprise separating the cell culture in a solid phase and in a liquid phase to obtain a supernatant. The supernatant can then be contacted with one or more adsorbent resins to which the compound(s) can bind, and the compound(s) can then be eluted as is known in the art. Alternatively, one or more ion exchange or reversed-phase chromatography columns can be used. Another option is to employ liquid-liquid extraction in an immiscible solvent, which may optionally be evaporated before precipitating the compound(s), or further liquid-liquid extraction may be employed.

The cell is preferably as defined herein.

In some embodiments, the method is for production of halogenated tryptophan(s) and further comprises a step of recovering the halogenated tryptophan(s).

In some embodiments, the method is for production of dihalogenated tryptophan(s) and further comprises a step of recovering the dihalogenated tryptophan(s).

In some embodiments, the method is for production of trihalogenated tryptophan(s) and further comprises a step of recovering the trihalogenated tryptophan(s).

In some embodiments, the method is for production of halogenated tryptamine(s) and further comprises a step of recovering the halogenated tryptamine(s).

In some embodiments, the method is for production of dihalogenated tryptamine(s) and further comprises a step of recovering the dihalogenated tryptamine(s).

In some embodiments, the method is for production of trihalogenated tryptamine(s) and further comprises a step of recovering the trihalogenated tryptamine(s).

In some embodiments, the method is for production of halogenated N-methyltryptamine, halogenated N,N-dimethyltryptamine and/or halogenated N,N,N-trimethyltryptamine, and further comprises a step of recovering the halogenated N-methyltryptamine, halogenated N,N-dimethyltryptamine and/or halogenated N,N,N-trimethyltryptamine.

In some embodiments, the method is for production of dihalogenated N-methyltryptamine, dihalogenated N,N-dimethyltryptamine and/or dihalogenated N,N,N-trimethyltryptamine, and further comprises a step of recovering the dihalogenated N-methyltryptamine, dihalogenated N,N-dimethyltryptamine and/or dihalogenated N,N,N-trimethyltryptamine.

In some embodiments, the method is for production of trihalogenated N-methyltryptamine, trihalogenated N,N-dimethyltryptamine and/or trihalogenated N,N,N-trimethyltryptamine, and further comprises a step of recovering the trihalogenated N-methyltryptamine, trihalogenated N,N-dimethyltryptamine and/or trihalogenated N,N,N-trimethyltryptamine.

Titers

The present methods are useful for producing halogenated tryptophans and derivatives thereof with high titers.

In some embodiments, 2-halogenated, 5-halogenated, 6-halogenated, or 7-halogenated tryptophan is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5-dihalogenated, 2,6-dihalogenated, 2,7-dihalogenated, 5,6-dihalogenated, 5,7-dihalogenated or 6,7-dihalogenated tryptophan is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5,6-trihalogenated, 2,5,7-trihalogenated, 2,6,7-trihalogenated or 5,6,7-trihalogenated tryptophan is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2-halogenated, 5-halogenated, 6-halogenated, or 7-halogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5-dihalogenated, 2,6-dihalogenated, 2,7-dihalogenated, 5,6-dihalogenated, 5,7-dihalogenated or 6,7-dihalogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5,6-trihalogenated, 2,5,7-trihalogenated, 2,6,7-trihalogenated or 5,6,7-trihalogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2-halogenated N-methyltryptamine, 2-halogenated N,N-dimethyltryptamine, 2-halogenated N,N,N-trimethyltryptamine, 5-halogenated N-methyltryptamine, 5-halogenated N,N-dimethyltryptamine, 5-halogenated N,N,N-trimethyltryptamine, 6-halogenated N-methyltryptamine, 6-halogenated N,N-dimethyltryptamine, 6-halogenated N,N,N-trimethyltryptamine, 7-halogenated N-methyltryptamine, 7-halogenated N,N-dimethyltryptamine, or 7-halogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5-dihalogenated N-methyltryptamine, 2,5-dihalogenated N,N-dimethyltryptamine, 2,5-dihalogenated N,N,N-trimethyltryptamine, 2,6-dihalogenated N-methyltryptamine, 2,6-dihalogenated N,N-dimethyltryptamine, 2,6-dihalogenated N,N,N-trimethyltryptamine, 2,7-dihalogenated N-methyltryptamine, 2,7-dihalogenated N,N-dimethyltryptamine, 2,7-dihalogenated N,N,N-trimethyltryptamine, 5,6-dihalogenated N-methyltryptamine, 5,6-dihalogenated N,N-dimethyltryptamine, 5,6-dihalogenated N,N,N-trimethyltryptamine, 5,7-dihalogenated N-methyltryptamine, 5,7-dihalogenated N,N-a dimethyltryptamine, 5,7-dihalogenated N,N,N-trimethyltryptamine, 6,7-dihalogenated N-methyltryptamine, 6,7-dihalogenated N,N-dimethyltryptamine, or 6,7-dihalogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, 2,5,6-trihalogenated N-methyltryptamine, 2,5,6-trihalogenated N,N-dimethyltryptamine, 2,5,6-trihalogenated N,N,N-trimethyltryptamine, 2,5,7-trihalogenated N-methyltryptamine, 2,5,7-trihalogenated N,N-dimethyltryptamine, 2,5,7-trihalogenated N,N,N-trimethyltryptamine, 2,6,7-trihalogenated N-methyltryptamine, 2,6,7-trihalogenated N,N-dimethyltryptamine, 2,6,7-trihalogenated N,N,N-trimethyltryptamine, 5,6,7-trihalogenated N-methyltryptamine, 5,6,7-trihalogenated N,N-dimethyltryptamine, or 5,6,7-trihalogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the total titer of all halogenated compounds produced is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more, wherein the total titer is the sum of the titers of the 2-halogenated, 5-halogenated, 6-halogenated, 7-halogenated, 2,5-dihalogenated, 2,6-dihalogenated, 2,7-dihalogenated, 5,6-dihalogenated, 5,7-dihalogenated, 6,7-dihalogenated, 2,5,6-trihalogenated, 2,5,7-trihalogenated, 2,6,7-trihalogenated and 5,6,7-trihalogenated tryptophans, tryptamines, N-methyltryptamines, N,N-dimethyltryptamines and N,N,N-trimethyltryptamines produced.

Methods for determining the titer are known in the art, for example by measuring the peak area from LC-MS analysis and comparing to the peak area of an authentic analytical standard of known concentration.

Halogenated Tryptophans and Derivatives Thereof Obtainable by the Present Methods

In one aspect, the present invention provides halogenated tryptophans and derivatives thereof obtainable by a method as disclosed herein.

In some embodiments, the present invention provides halogenated tryptophans obtainable by the method described herein. In some embodiments, the present invention provides dihalogenated tryptophans obtainable by the method described herein. In some embodiments, the present invention provides trihalogenated tryptophans obtainable by the method described herein. In some embodiments, the present invention provides halogenated tryptamines obtainable by the method described herein. In some embodiments, the present invention provides dihalogenated tryptamines obtainable by the method described herein. In some embodiments, the present invention provides trihalogenated tryptamines obtainable by the method described herein. In some embodiments, the present invention provides halogenated N-methyltryptamines obtainable by the method described herein. In some embodiments, the present invention provides dihalogenated N-methyltryptamines obtainable by the method described herein. In some embodiments, the present invention provides trihalogenated N-methyltryptamines obtainable by the method described herein. In some embodiments, the present invention provides halogenated N,N-dimethyltryptamines obtainable by the method described herein. In some embodiments, the present invention provides dihalogenated N,N-dimethyltryptamines obtainable by the method described herein. In some embodiments, the present invention provides trihalogenated N,N-dimethyltryptamines obtainable by the method described herein. In some embodiments, the present invention provides halogenated N,N,N-trimethyltryptamines obtainable by the method described herein. In some embodiments, the present invention provides dihalogenated N,N,N-trimethyltryptamines obtainable by the method described herein. In some embodiments, the present invention provides trihalogenated N,N,N-trimethyltryptamines obtainable by the method described herein.

Nucleic Acid Constructs

Also provided herein are nucleic acid constructs useful for engineering a cell capable of producing halogenated tryptophan or derivatives thereof as described above. The present nucleic acid constructs may be provided as one or more nucleic acid molecules or polynucleotides, for example they may be comprised in one or more vectors. Such nucleic acids may be introduced in the cell by methods known in the art.

It will be understood that throughout the present disclosure, the term ‘nucleic acid encoding an activity’ shall refer to a nucleic acid molecule capable of encoding a peptide, a protein or a fragment thereof having said activity. Such nucleic acid molecules may be open reading frames or genes or fragments thereof.

In some aspects, the present invention provides a nucleic acid construct for modifying a cell, said construct comprising at least one of:
a polynucleotide encoding a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-5-halogenase (EC 1.14.19.58), preferably a heterologous tryptophan-5-halogenase such as SrPyrH (SEQ ID NO: 32) or a functional variant thereof having at least 80% homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a polynucleotide encoding a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a polynucleotide encoding a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,

and optionally a polynucleotide encoding a flavin reductase, preferably a heterologous flavin reductase (EC 1.5.1.30), such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nucleic acid further comprises a polynucleotide encoding a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one aspect, the present invention provides a nucleic acid construct for modifying a cell, said construct comprising at least one of:
a polynucleotide encoding a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-5-halogenase (EC 1.14.19.58), preferably a heterologous tryptophan-5-halogenase such as SrPyrH (SEQ ID NO: 32) or a functional variant thereof having at least 80% homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a polynucleotide encoding a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a polynucleotide encoding a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally a polynucleotide encoding a flavin reductase, preferably a heterologous flavin reductase (EC 1.5.1.30), such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,

and also comprising a polynucleotide encoding a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nucleic acid further comprises a polynucleotide encoding an indole N-methyltransferase (EC 2.1.1.49), preferably a heterologous indole N-methyltransferase such as OcINMT (SEQ ID NO: 36) or a functional variant thereof having at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one aspect, the present invention provides a nucleic acid construct for modifying a cell, said construct comprising at least one of:
a polynucleotide encoding a tryptophan-2-halogenase (EC 1.14.14), preferably a heterologous tryptophan-2-halogenase such as CcCmdE (SEQ ID NO: 48) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-5-halogenase (EC 1.14.19.58), preferably a heterologous tryptophan-5-halogenase such as SrPyrH (SEQ ID NO: 32) or a functional variant thereof having at least 80% homology, such as at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
a polynucleotide encoding a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-6-halogenase is SttH (SEQ ID NO: 33) or a functional variant thereof having at least 80% homology thereto,
a polynucleotide encoding a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, more preferably the tryptophan-7-halogenase is LaRebH (SEQ ID NO: 34) or a functional variant thereof having at least 80% homology thereto, or
a polynucleotide encoding a tryptophan halogenase, preferably a heterologous tryptophan halogenase such as DdChlA (SEQ ID NO: 52), or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and optionally a polynucleotide encoding a flavin reductase, preferably a heterologous flavin reductase (EC 1.5.1.30), such as LaRebF (SEQ ID NO: 35) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,

also comprising a polynucleotide encoding a tryptophan decarboxylase (EC 4.1.1.105), preferably a heterologous tryptophan decarboxylase such as CrTDC (SEQ ID NO: 1) or a functional variant thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto,
and additionally comprising a polynucleotide encoding an indole N-methyltransferase (EC 2.1.1.49), preferably a heterologous indole N-methyltransferase such as OcINMT (SEQ ID NO: 36) or a functional variant thereof having at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan-2-halogenase (EC 1.14.14) is a heterologous tryptophan-2-halogenase such as CcCmdE (SEQ ID NO: 48) or a functional variant thereof having at least 80% homology thereto, and encoded by a polynucleotide comprising or consisting of SEQ ID NO: 49 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan-5-halogenase (EC 1.14.19.58) is a heterologous tryptophan-5-halogenase such as SrPyrH (SEQ ID NO: 32) or a functional variant thereof having at least 80% homology thereto, and encoded by a polynucleotide comprising or consisting of SEQ ID NO: 37 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan-6-halogenase (EC 1.14.19.59) is a heterologous tryptophan-6-halogenase such as SttH (SEQ ID NO: 33), SaThaI (SEQ ID NO: 51), or KtzR (SEQ ID NO: 54), or a functional variant thereof having at least 80% homology thereto, and encoded by a polynucleotide comprising or consisting of SEQ ID NO: 38, SEQ ID NO: 56, or SEQ ID NO: 59, respectively, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the tryptophan-6-halogenase is SttH (SEQ ID NO: 33) or a functional variant thereof having at least 80% homology thereto, and encoded by a polynucleotide comprising or consisting of SEQ ID NO: 38 or a homologue thereof having at least 80% thereto.

In some embodiments, the tryptophan-7-halogenase (EC 1.14.19.9) is a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology thereto, and encoded by a polynucleotide comprising or consisting of SEQ ID NO: 39. SEQ ID NO: 55, or SEQ ID NO: 58, respectively, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the tryptophan-7-halogenase is LaRebH (SEQ ID NO: 34) or a functional variant thereof having at least 80% homology thereto, and encoded by a polynucleotide comprising or consisting of SEQ ID NO: 39.

In some embodiments, the tryptophan halogenase is a heterologous tryptophan halogenase such as DdChlA (SEQ ID NO: 52) or a functional variant thereof having at least 80% homology thereto, and encoded by a polynucleotide comprising or consisting of SEQ ID NO: 57 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the flavin reductase (EC 1.5.1.30) is a heterologous flavin reductase such as LaRebF (SEQ ID NO: 35) or a functional variant thereof having at least 80% homology thereto, and encoded by a polynucleotide comprising or consisting of SEQ ID NO: 40 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the tryptophan decarboxylase (EC 4.1.1.105) is a heterologous tryptophan decarboxylase such as CrTDC (SEQ ID NO: 1) or a functional variant thereof having at least 80% homology thereto, and encoded by a polynucleotide comprising or consisting of SEQ ID NO: 6 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the indole N-methyltransferase (EC 2.1.1.49) is a heterologous indole N-methyltransferase such a OcINMT (SEQ ID NO: 36) or a functional variant thereof having at least 80% homology thereto, and encoded by a polynucleotide comprising or consisting of SEQ ID NO: 41 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, one or more of the above polynucleotide(s) is/are codon-optimized for said cell.

In some embodiments, each of the nucleic acids encoding each of the present activities, i.e. a tryptophan halogenase, a tryptophan-2-halogenase, a tryptophan-5-halogenase, a tryptophan-6-halogenase or a tryptophan-7-halogenase, a flavin reductase, a tryptophan decarboxylase, and an indole N-methyltransferase, may be designed to be integrated within the genome of the cell or they may be within one or more vectors comprised within the cell.

In some embodiments, one or more of the nucleic acids encoding each of the present activities may be integrated in the genome of said cell. Methods for integrating a nucleic acid are well known in the art. Thus in some embodiments the activity of interest is encoded by introduction of a heterologous nucleic acid in the cell. The heterologous nucleic acid encoding said activity may be codon-optimized, or may comprise features that can help improve the activity. Such modifications include, but are not limited to, the introduction of localization signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognize that such methods include, but are not limited to: cloning and homologous recombination-based methods. Cloning methods may involve the design and construction of a plasmid e.g. in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the cell.

The nucleic acids encoding the activities of interest may be present in high copy number.

In some embodiments, the nucleic acid construct further comprises or consists of one or more vectors, such as an integrative vector or a replicative vector. In some embodiments, the vector is a high copy replicative vector.

Each of the nucleic acid sequences comprised within the present nucleic acid constructs may be present in multiple copies. In some embodiments, at least one of the nucleic acid sequences is present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies. In some embodiments, all of the nucleic acid sequences are present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies.

The nucleic acid constructs may, in addition to the polynucleotides described above, also comprise additional polynucleotides useful for introducing additional modifications in the cell, to obtain cells as described in “Other modifications”. Designing such additional polynucleotides can be performed as is known in the art.

The nucleic acid constructs may be a PCR product or a synthetic DNA molecule.

Kit of Parts

Also provided herein is a kit of parts comprising a cell, and/or a nucleic acid construct as described herein, and instructions for use.

In some embodiments, the kit comprises a cell that can be used in the methods described herein. In other embodiments, the kit comprises a nucleic acid construct that can be used to engineer a cell useful for the methods described herein. In some embodiments, the kit comprises a cell and a nucleic acid construct as described herein.

In some embodiments, the kit comprises a cell capable of producing one or more mono-, di- and/or tri-halogenated tryptophans, wherein the cell expresses one or more tryptophan halogenases and optionally a flavin reductase. In some embodiments, the kit comprises a cell capable of producing one or more mono-, di-, and/or tri-halogenated tryptamines, wherein the cell expresses one or more tryptophan halogenases, a tryptophan decarboxylase and optionally a flavin reductase. In some embodiments, the kit comprises a cell capable of producing one or more mono-, di-, and/or tri-halogenated N-methyltryptamines, one or more mono-, di-, and/or tri-halogenated N,N-dimethyltryptamines, and/or one or more mono-, di-, and/or tri-halogenated N,N,N-trimethyltryptamines, wherein the cell expresses one or more tryptophan halogenases, a tryptophan decarboxylase, an indole N-methyltransferase and optionally a flavin reductase. The cell may be further modified as detailed in “Other modifications”.

In some embodiments, the kit comprises a nucleic construct comprising a polynucleotide encoding a tryptophan halogenase. In some embodiments, the kit comprises a nucleic construct comprising a polynucleotide encoding a tryptophan halogenase and a polynucleotide encoding a tryptophan decarboxylase. In some embodiments, the kit comprises a nucleic construct comprising a polynucleotide encoding a tryptophan halogenase, a polynucleotide encoding a tryptophan decarboxylase and a polynucleotide encoding an indole N-methyltransferase. Additionally, any of the previously cited may also comprise a polynucleotide encoding a flavin reductase.

In some embodiments, the kit comprises the nucleic acid constructs described herein and the cell to be modified. In some embodiments, the cell to be modified is a yeast cell. In a preferred embodiment, the cell to be modified is a Saccharomyces cerevisiae cell or a Yarrowia lipolytica cell.

In some embodiments, the kit comprises the cell and a nucleic acid construct as described herein.

Methods and Cells for Production of Methylated Tryptamine

The present disclosure also relates to methods for producing methylated tryptamines, in particular N-methyltryptamine, N,N-dimethyltryptamine and/or N,N,N-trimethyltryptamine. The term “methylated tryptamine” herein refers to tryptamine substituted with one or more methyl groups, such as two or more, such as three or more methyl groups. Throughout the present disclosure, it will be understood that the cells can produce the compounds of interest listed herein when incubated in a cultivation medium under conditions that enable the cell to grow and produce the desired compound. From the description of the production host cells provided herein, and knowing the type of host cell used, the skilled person will not have difficulties in identifying suitable cultivation media and conditions to achieve production. In particular, the cultivation may be performed aerobically or anaerobically, at temperatures and at pH suitable for supporting growth of the cell. The cultivation medium should include the required nutrients, and may be supplemented with precursors as applicable. The time of cultivation will vary depending on which cell is used, but can easily be adapted by the skilled person.

Herein is also provided a cell capable of producing N-methyltryptamine, N,N-dimethyltryptamine and/or N,N,N-trimethyltryptamine, preferably wherein the cell is a microorganism or a plant cell, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine; and
an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase is capable of converting tryptamine to N-methyltryptamine, to N,N-dimethyltryptamine, and/or to N,N,N-trimethyltryptamine,

whereby the cell is capable of producing N-methyltryptamine, N,N-dimethyltryptamine, and/or N,N,N-trimethyltryptamine.

The cell may be as described herein above, in particular the cell may be a yeast cell for example a S. cerevisiae cell, or a cell as described in the section “Other organisms” above. Any of the modifications otherwise described herein, in particular in the section “Other modifications”, may also be applied to cells producing methylated tryptamines.

Such cells are useful in methods for producing methylated tryptamine. Also provided herein is a method for producing N-methyltryptamine, N,N-dimethyltryptamine and/or

N,N,N-trimethyltryptamine in a cell, preferably wherein the cell is a microorganism or a plant cell, said method comprising the steps of providing a cell and incubating said cell in a medium, wherein the cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the tryptophan decarboxylase is capable of converting tryptophan to tryptamine; and
an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the indole N-methyltransferase is capable of converting tryptamine to N-methyltryptamine, to N,N-dimethyltryptamine, and/or to N,N,N-trimethyltryptamine.

Recovering the Methylated Tryptamines

Thus the present methods may comprise a further step of recovering the compounds obtained by the methods disclosed herein. Methods for recovering the products obtained by the present invention are known in the art, for example organic solvent extraction followed by lyophilisation and purification by preparative HPLC or similar column purification techniques. Methods for recovering the products obtained by the present invention are known in the art, for example organic solvent extraction followed by lyophilisation and purification by preparative HPLC or similar column purification techniques. For example, the step of recovering the compound(s) may comprise separating the cell culture in a solid phase and in a liquid phase to obtain a supernatant. The supernatant can then be contacted with one or more adsorbent resins to which the compound(s) can bind, and the compound(s) can then be eluted as is known in the art. Alternatively, one or more ion exchange or reversed-phase chromatography columns can be used. Another option is to employ liquid-liquid extraction in an immiscible solvent, which may optionally be evaporated before precipitating the compound(s), or further liquid-liquid extraction may be employed.

The yeast cell is preferably as defined herein.

In some embodiments, the method is for production of N-methyltryptamine, N,N-dimethyltryptamine and/or N,N,N-trimethyltryptamine and further comprises a step of recovering the N-methyltryptamine, N,N-dimethyltryptamine and/or N,N,N-trimethyltryptamine.

Titers

The above cells can produce N-methyltryptamine, N,N-dimethyltryptamine and/or N,N,N-trimethyltryptamine with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell produces N-methyltryptamine with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell produces N,N-dimethyltryptamine with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the cell produces N,N,N-trimethyltryptamine with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

In some embodiments, the titer of methylated tryptamine, i.e. the sum of the titers of N-methyltryptamine, N,N-dimethyltryptamine and N,N,N-trimethyltryptamine produced by the cell, is at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

Also provided herein is N-methyltryptamine, N,N-dimethyltryptamine and/or N,N-dimethyltryptamine obtainable by the methods disclosed herein.

Nucleic Acid Constructs

Also provided herein are nucleic acid constructs useful for engineering cells capable of producing methylated tryptamine.

Such constructs comprise:
a polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, such as a polynucleotide comprising or consisting of SEQ ID NO: 6 or a homologue thereof having at least 80% homology, such as at least 85%, such as at least 90%, such as at least 95% homology thereto
a polynucleotide encoding an indole N-methyltransferase, preferably a heterologous indole N-methyltransferase (EC 2.1.1.49), such as OcINMT (SEQ ID NO: 36) or a functional variant thereof having at least 80% homology, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, such as a polynucleotide comprising or consisting of SEQ ID NO: 36 or a homologue thereof having at least 80% homology, such as at least 85%, such as at least 90%, such as at least 95% homology thereto.

The polynucleotide encoding the tryptophan decarboxylase may herein be referred to as the “first polynucleotide”. The polynucleotide encoding the indole N-methyltransferase may be referred to as the “seventh polynucleotide”. This does not imply that the construct comprises eight polynucleotides in total; in some embodiments the cell comprises only the first and the seventh polynucleotides.

The first polynucleotide is as described herein above.

The seventh polynucleotide is as described herein above.

In some embodiments, one or more of the first and seventh polynucleotide(s) is/are codon-optimised for said yeast cell.

In some embodiments, each of the nucleic acids encoding each of the present activities, i.e. a tryptophan decarboxylase or an indole N-methyltransferase, may be designed to be integrated within the genome of the yeast cell or they may be within one or more vectors comprised within the yeast cell.

In some embodiments, one or more of the nucleic acids encoding each of the present activities may be integrated in the genome of said yeast cell. Methods for integrating a nucleic acid are well known in the art. Thus in some embodiments the activity of interest is encoded by introduction of a heterologous nucleic acid in the yeast cell. The heterologous nucleic acid encoding said activity may be codon-optimised, or may comprise features that can help improve the activity. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid e.g. in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the activities of interest may be present in high copy number.

In some embodiments, the nucleic acid construct further comprises or consists of one or more vectors, such as an integrative vector or a replicative vector. In some embodiments, the vector is a high copy replicative vector.

Each of the nucleic acid sequences comprised within the present nucleic acid constructs may be present in multiple copies. In some embodiments, at least one of the nucleic acid sequences is present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies. In some embodiments, all of the nucleic acid sequences are present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies.

The nucleic acid constructs may, in addition to the first and seventh polynucleotides described above, also comprise additional polynucleotides useful for introducing additional modifications in the yeast cell, to obtain cells as described in “Other modifications”. Designing such additional polynucleotides can be performed as is known in the art.

The nucleic acid constructs may be a PCR product or a synthetic DNA molecule.

Kit of Parts

Also provided herein is a kit of parts comprising a cell, for example a yeast cell as described herein, or any other cell described herein, and/or a nucleic acid construct as described herein, and instructions for use.

In some embodiments, the kit comprises a yeast cell that can be used in the methods for producing methylated tryptamines described herein. In other embodiments, the kit comprises a nucleic acid construct that can be used to engineer a yeast cell useful for the methods for producing methylated tryptamines described herein. In some embodiments, the kit comprises a yeast cell and a nucleic acid construct as described herein.

In some embodiments, the kit comprises a yeast cell capable of producing methylated tryptamines, in particular N-methyltryptamine, N,N-dimethyltryptamine and/or N,N,N-trimethyltryptamine, wherein the yeast cell expresses a tryptophan decarboxylase and an indole N-methyltransferase. The yeast cell may be further modified as detailed in “Other modifications”.

In some embodiments, the kit comprises a nucleic construct comprising a first polynucleotide encoding a tryptophan decarboxylase and a seventh polynucleotide encoding an indole N-methyltransferase.

In some embodiments, the kit comprises the nucleic acid construct as described herein and the yeast cell to be modified. In some embodiments, the yeast cell to be modified is a Saccharomyces cerevisiae cell or a Yarrowia lipolytica cell.

In some embodiments, the kit comprises the yeast cell and a nucleic acid construct as described herein.

Advanced Microbiome Therapy

Many diseases relate to a condition referred to as dysbiosis, a microbial imbalance in the gut associated with a bloom of pathobionts, loss of commensals, and loss of diversity within the gut. Given the implications of the gut microbiome in disease, means for modulating the gut microbiome have been explored with the goal of lowering disease prevalence. Among the approaches tested is the administration of faecal microbial transplants for the treatment of diseases ranging from irritable bowel syndrome to chronic fatigue syndrome. However, such faecal microbial transplants have recently been linked to multiple cases of serious and fatal adverse events due to the transfer of drug-resistant bacteria. In view of concerns as to the safety and efficacy of this treatment, alternative methods for modulating the gut microbiome are needed.

One such method is through the use of prebiotics, which are non-digestible substrates associated with an increased density of health-promoting microorganisms. As such, prebiotics serve as a less invasive and short-term mechanism for modulating the gut microbiome for health benefits. Overall, consumption of prebiotics such as inulin, fructo-oligosaccharides, and galacto-oligosaccharides increases the density of beneficial microorganisms, in particular bacteria such as Bifidobacterium and Lactobacilli species.

Probiotics, living organisms that are beneficial to health, have also been shown to help modulate the gut microbiome in order to improve health. As with prebiotics, probiotics are specifically used to alter the gut environment. However, instead of seeking to up-regulate beneficial bacteria by providing prebiotic substrates, such as dietary fibers, probiotics are used to directly introduce beneficial strains. Probiotics function by either directly interacting with the host via chemical and physical signals or by affecting the make-up of the gut microbial community. Previously, probiotics have been useful in treating obesity, diabetes, inflammation, cancer, allergies, and many other ailments.

The success of probiotics in benefitting the gut environment, as well as human health as a whole, has led to a new generation of probiotics engineered to augment the innate benefits of probiotics through a wide range of mechanisms such as the production of therapeutics. These next generation probiotics are often referred to as smart probiotics, living therapeutics, or advanced microbial therapeutics. In such systems, microbial production of therapeutics allows for a continuous and inexpensive supply of molecules such as hormones, interleukins, and antibodies. With a potential for secreting a range of molecules, these living therapeutics have a wide scope of possibilities stretching far beyond the already important role of gut microbes. As some therapeutics are unstable or require high doses, utilizing engineered microbials may be a superior alternative to traditional drug delivery as the microbe-produced therapeutic avoids exposure to the harsh acidic conditions of the upper gastrointestinal tract. Additionally, with an ever-expanding toolbox of sensors, killswitches, memory circuits, etc., these microorganisms can be fine-tuned to better secrete therapeutics, sense signals within the gut environment, and respond to physiological changes.

Accordingly, the present disclosure also provides for the yeast cells disclosed herein to be used as probiotics. These yeast cells have been engineered to produce one or more compounds as described above, where the one or more compounds is selected from the group consisting of: a halogenated tryptophan, a halogenated tryptamine, a halogenated, di-halogenated or tri-halogenated N-methyltryptamine, a halogenated, di-halogenated or tri-halogenated N,N-dimethyltryptamine, a halogenated, di-halogenated or tri-halogenated N,N,N-trimethyltryptamine, N-methyltryptamine, N,N-dimethyltryptamine and/or N,N,N-trimethyltryptamine, norbaeocystin, baeocystin, norpsilocin, psilocybin, psilocin, aeruginascin, dephosphorylated aeruginascin, N-acetyl-4-hydroxytryptamine, and derivatives thereof. Any of the yeast cells described herein above may be used as probiotic, particularly in the context of advance microbiome therapy.

The yeast cell may be provided as a composition comprising the yeast cell, such as a pharmaceutical composition comprising the yeast cell.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell, a Saccharomyces boulardii cell or a Yarrowia lipolytica cell. Most preferably, the yeast cell is a Saccharomyces boulardii cell.

Any of the yeast cells of the present disclosure, or any composition or pharmaceutical composition comprising said yeast cells, may thus be used as a probiotic. Without being bound by theory, it is envisaged that upon administration to the subject, the yeast cells release the one or more compounds produced by the yeast cell, in particular one or more of a halogenated tryptophan, a halogenated tryptamine, a halogenated, di-halogenated or tri-halogenated N-methyltryptamine, a halogenated, di-halogenated or tri-halogenated N,N-dimethyltryptamine, a halogenated, di-halogenated or tri-halogenated N,N,N-trimethyltryptamine, N-methyltryptamine, N,N-dimethyltryptamine and/or N,N,N-trimethyltryptamine, norbaeocystin, baeocystin, norpsilocin, psilocybin, psilocin, aeruginascin, dephosphorylated aeruginascin, N-acetyl-4-hydroxytryptamine, and derivatives thereof. Yeast cells producing said compounds have been described in detail herein above.

In addition to the modifications described herein, the yeast cell may be further engineered to allow for biocontainment of the yeast cells. Thus in some embodiments, the yeast cell further comprises means for biocontainment. Such means may include a conditional suicide system or a genetic switch system, which trigger inactivation or destruction of the yeast cell upon activation, for example through engineered auxotrophy. Other means for biocontainment include kill switches, where death of the yeast cell can be induced by the presence of an inducer. Alternatively, interruption of administration can also be used in order to prevent the microorganisms from settling in the intestinal tract in the long term.

Preferably, the yeast cell, the composition or the pharmaceutical composition comprising the yeast cell, are for oral administration. The skilled person will know how to formulate the yeast cell, the composition or the pharmaceutical composition for such administration.

The yeast cells and compositions may be useful for treating or preventing a disorder or a disease, particularly disorders and diseases where it is envisioned that one of the above compounds, i.e. a halogenated tryptophan, a halogenated tryptamine, a halogenated, di-halogenated or tri-halogenated N-methyltryptamine, a halogenated, di-halogenated or tri-halogenated N,N-dimethyltryptamine, a halogenated, di-halogenated or tri-halogenated N,N,N-trimethyltryptamine, N-methyltryptamine, N,N-dimethyltryptamine and/or N,N,N-trimethyltryptamine, norbaeocystin, baeocystin, norpsilocin, psilocybin, psilocin, aeruginascin, dephosphorylated aeruginascin, N-acetyl-4-hydroxytryptamine, and derivatives thereof, is expected to have a therapeutic effect. In preferred embodiments, the compound is psilocybin or psilocin.

Relevant diseases and disorders include depression, such as major depressive disorder or treatment-resistant depression, anxiety disorders, obsessive-compulsive disorder, post-traumatic stress disorder, substance addiction or dependence such as alcohol or tobacco addiction or dependence, migraine and headache, preferably chronic migraines and chronic headaches.

The yeast cell, the composition or the pharmaceutical composition, are to be administered to a subject in need thereof. Subjects in need thereof include subjects suffering of, suspected of suffering of, or at risk of suffering of any one of the above listed diseases and disorders. The subject may be a mammal, such as a human, a farm animal such as a pig, a cow, a sheep, poultry, or a pet, in particular mammalian pets such as cats and dogs.

The present yeast cells and compositions are also expected to be useful in methods for increasing empathy and/or creativity of the subject to which they are administered.

Thus is also provided herein a method for increasing empathy and/or creativity of a subject, comprising administering to the subject any of the yeast cells described herein, or a composition or pharmaceutical composition comprising any of the yeast cells described herein.

The yeast cell, the composition or the pharmaceutical composition are preferably administered in such a dosage that only minute amounts of the one or more compounds are released in the intestinal tract of the subject. In some embodiments, the one or more compounds is delivered to the subject in an amount in the range of 1 ng to 1 mg, such as between 1 ng and 750 μg, such as between 5 ng and 500 μg, such as between 10 ng and 250 μg, such as between 25 ng and 100 μg, such as between 50 ng and 75 μg, such as between 75 ng and 50 μg, such as between 50 ng and 25 μg, such as between 75 ng and 10 μg, such as between 100 ng and 7.5 μg, such as between 250 ng and 5 μg, such as between 500 ng and 2.5 μg, such as between 750 ng and 1 μg.

The yeast cell, the composition or the pharmaceutical composition may thus be administered one to five times a day, such as once daily, twice daily, thrice daily, four times daily or five times daily, or every second day, every third day, once a week, every second week, or once a month.

### EXAMPLES

### Example 1: Materials and Methods

Strains Media and Maintenance

All S. cerevisiae strains used in this study (Table 1) were derived from the CEN.PK strain family background. Frozen stocks of E. coli and S. cerevisiae were prepared by addition of glycerol (30% (v/v)) to exponentially growing cells and aseptically storing 1 mL aliquots at −80° C. Cultures were grown in synthetic medium according to the following recipes. Synthetic medium was prepared with 7.5 g/L (NH₄)₂SO₄, 14.4 g/L KH₂PO₄, 0.5 g/L MgSO47H2O and appropriate growth factors. Synthetic complete media minus uracil supplementation (SC-Ura) was prepared with 3 g/L synthetic complete minus uracil powder and 5 g/L (NH₄)₂SO₄. YP medium was prepared with 10 g/L yeast extract and 20 g/L peptone. In all cases unless stated otherwise, 20 g/L glucose was added. Synthetic feed-in-time (FIT) media was prepared by adding 60 g/L EnPump 200 substrate (polysaccharide) and 0.3% reagent A (hydrolyzing enzyme) to synthetic media. Media was supplemented with 200 mg/L G418 and 100 mg/L nourseothricin when required. E. coli strains were grown in Luria-Bertani (LB) media and supplemented with 100 mg/L ampicillin when required. Agar plates were prepared as described above but with the addition of 20 g/L agar.

TABLE 1
Strains used in the study

| | Parental | Added DNA | Relevant | |
|---|---|---|---|---|
| Name | strain | element | genotype | Source |
| CEN.PK113- | — | — | MATa Δura3 | Entian |
| 5D | | | HIS3 LEU2 | and |
| | | | TRP1 MAL2-8c | Kötter, |
| | | | SUC2 | 2007 |
| ST8251 | CEN.PK113- | pCfB2312 | MATa Δura3 | Jessop- |
| | 5D | | HIS3 LEU2 | Fabre |
| | | | TRP1 MAL2-8c | et al., |
| | | | SUC2 + | 2016 |
| | | | pCfB2312 | |
| | | | (Cas9) | |
| ST8940 | ST8251 | pCfB8794 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK | |
| ST8983 | ST8940 | BB3923 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| ST9016 | ST8983 | BB3939 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr | |
| ST9020 | ST8983 | BB3940 | MATa Δura3 | This |
| | | | HI S3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF2−> | |
| | | | PcCpr | |
| ST9109 | ST8983 | BB3953 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-2:: CYB5- | |
| | | | AtAtr2 | |
| ST9179 | ST9016 | PR-23852 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr ric1:: | |
| ST9316 | ST9179 | BB4020 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr ric1:: | |
| | | | X-4:: ARO1- | |
| | | | ARO2 | |
| ST9318 | ST9316 | pCfB9074 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr ric1:: | |
| | | | X-4:: ARO1- | |
| | | | ARO2 XII-4:: | |
| | | | ARO4^{K229L}- | |
| | | | TRP2^{S65R, S76L} | |
| ST9326 | CEN.PK113- | pCfB255 | MATa Δura3 | This |
| | 5D | | HIS3 LEU2 | study |
| | | | TRP1 MAL2-8c | |
| | | | SUC2 X-2:: | |
| | | | URA3 | |
| ST9327 | ST8983 | pCfB255 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | X-2:: URA3 | |
| ST9328 | ST9016 | pCfB255 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr X-2:: | |
| | | | URA3 | |
| ST9329 | ST9020 | pCfB255 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF2−> | |
| | | | PcCpr X-2:: | |
| | | | URA3 | |
| ST9330 | ST9109 | pCfB255 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-2:: CYB5- | |
| | | | AtAtr2 X-2:: | |
| | | | URA3 | |
| ST9334 | ST9016 | pCfB9013 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr Ty- | |
| | | | 4::PcPsiK | |
| ST9335 | ST9016 | pCfB8796 | MATa Δura3 | This |
| | | | HIS3 LEU2 | study |
| | | | TRP1 MAL2-8c | |
| | | | SUC2 XII-5:: | |
| | | | PcPsiM- | |
| | | | PcPsiK XI-3:: | |
| | | | CrTdc-PcPsiH | |
| | | | XI-1:: pTEF1−> | |
| | | | PcCpr Ty- | |
| | | | 4::PcPsiM | |
| ST9337 | ST8251 | pCfB8881 | MATa Δura3 | This |
| | | | HIS3 LEU2 | study |
| | | | TRP1 MAL2-8c | |
| | | | SUC2 XI-3:: | |
| | | | CrTdc | |
| ST9346 | ST9337 | pCfB9073 | MATa Δura3 | This |
| | | | HIS3 LEU2 | study |
| | | | TRP1 MAL2-8c | |
| | | | SUC2 XI-3:: | |
| | | | CrTdc XI-1:: | |
| | | | pTEF1−> | |
| | | | PcCpr-PcPsiH | |
| ST9442 | ST9346 | pCfB9111 | MATa Δura3 | This |
| | | | HIS3 LEU2 | study |
| | | | TRP1 MAL2-8c | |
| | | | SUC2 XI-3:: | |
| | | | CrTdc XI-1:: | |
| | | | pTEF1−> | |
| | | | PcCpr- | |
| | | | PcPsiH Ty-4:: | |
| | | | BtAANAT | |
| ST9647 | ST9337 | pCfB9624 | MATa Δura3 | This |
| | | (TyOcINMT) | HIS3 LEU2 | study |
| | | | TRP1 MAL2-8c | |
| | | | SUC2 XI-3:: | |
| | | | CrTdc Ty-4:: | |
| | | | OcINMT | |
| ST9733 | ST9316 | BB4182 | MATa Δura3 | This |
| | | (SPE2_KO) | HIS3 LEU2 | study |
| | | | TRP1 MAL2-8c | |
| | | | SUC2 XII-5:: | |
| | | | PsiM-PsiK XI- | |
| | | | 3:: CrTdc- | |
| | | | PcPsiH XI-1:: | |
| | | | pTEF1−> | |
| | | | PcCPR ric1:: | |
| | | | X-4:: ARO1- | |
| | | | ARO2 spe2:: | |
| ST9734 | ST9316 | BB4183 | MATa Δura3 | This |
| | | (ERG4_KO) | HIS3 LEU2 | study |
| | | | TRP1 MAL2-8c | |
| | | | SUC2 XII-5:: | |
| | | | PsiM-PsiK XI- | |
| | | | 3:: CrTdc- | |
| | | | PcPsiH XI-1:: | |
| | | | pTEF1−> | |
| | | | PcCPR ric1:: | |
| | | | X-4:: ARO1- | |
| | | | ARO2 erg4:: | |
| ST9328 + | ST9328 | pCfB9120(XII- | MATa Δura3 | This |
| POS5 | | 1MF: POS5) | HIS3 LEU2 | study |
| | | | TRP1 MAL2-8c | |
| | | | SUC2 XII-5:: | |
| | | | PsiM-PsiK XI- | |
| | | | 3:: CrTdc- | |
| | | | PcPsiH XI-1:: | |
| | | | pTEF1−> | |
| | | | PcCPR X-2:: | |
| | | | URA3 XII-1:: | |
| | | | POS5 | |
| ST9759 | ST7574 | pCfB9331(XI- | MATa URA3 | This |
| | | 1MF: SrPyrH- | HIS3 LEU2 | study |
| | | LaRebF) | TRP1 MAL2-8c | |
| | | | SUC2 XI-1:: | |
| | | | SrPyrH- | |
| | | | LaRebF | |
| ST9760 | ST7574 | pCfB9332(XI- | MATa URA3 | This |
| | | 1MF: SrPyrH) | HIS3 LEU2 | study |
| | | | TRP1 MAL2-8c | |
| | | | SUC2 XI-1:: | |
| | | | SrPyrH | |
| ST9761 | ST7574 | pCfB9333(XII- | MATa URA3 | This |
| | | 5: SttH- | HIS3 LEU2 | study |
| | | LaRebF) | TRP1 MAL2-8c | |
| | | | SUC2 XII-5:: | |
| | | | Stth-LaRebF | |
| ST9762 | ST7574 | pCfB9334(XII- | MATa URA3 | This |
| | | 5: SttH) | HIS3 LEU2 | study |
| | | | TRP1 MAL2-8c | |
| | | | SUC2 XII-5:: | |
| | | | Stth | |
| ST9336 | ST7574 | pCfB8881(XI- | MATa URA3 | This |
| | | 3MF: crTDC) | HIS3 LEU2 | study |
| | | | TRP1 MAL2-8c | |
| | | | SUC2 XI-3:: | |
| | | | CrTdc | |
| ST9763 | ST9761 | pCfB9332(XI- | MATa URA3 | This |
| | | 1MF: SrPyrH) | HIS3 LEU2 | study |
| | | | TRP1 MAL2-8c | |
| | | | SUC2 XII-5:: | |
| | | | Stth-LaRebF | |
| | | | XI-1:: SrPyrH | |
| ST9764 | ST9336 | pCfB9331(XI- | MATa URA3 | This |
| | | 1MF: SrPyrH- | HIS3 LEU2 | study |
| | | LaRebF), | TRP1 MAL2-8c | |
| | | | SUC2 XI-3:: | |
| | | | CrTdc XI-1:: | |
| | | | SrPyrH- | |
| | | | LaRebF | |
| ST9765 | ST9336 | pCfB9332(XI- | MATa URA3 | This |
| | | 1MF: SrPyrH), | HIS3 LEU2 | study |
| | | | TRP1 MAL2-8c | |
| | | | SUC2 XI-3:: | |
| | | | CrTdc XI-1:: | |
| | | | SrPyrH | |
| ST9766 | ST9336 | pCfB9333(XII- | MATa URA3 | This |
| | | 5: SttH- | HIS3 LEU2 | study |
| | | LaRebF), | TRP1 MAL2-8c | |
| | | | SUC2 XI-3:: | |
| | | | CrTdc XII-5:: | |
| | | | Stth-LaRebF | |
| ST9767 | ST9336 | pCfB9334(XII- | MATa URA3 | This |
| | | 5: SttH), | HIS3 LEU2 | study |
| | | | TRP1 MAL2-8c | |
| | | | SUC2 XI-3:: | |
| | | | CrTdc XII-5:: | |
| | | | Stth | |
| ST9768 | ST9766 | pCfB9332(XI- | MATa URA3 | This |
| | | 1MF: SrPyrH) | HIS3 LEU2 | study |
| | | | TRP1 MAL2-8c | |
| | | | SUC2 XI-3:: | |
| | | | CrTdc XII-5:: | |
| | | | Stth-LaRebF | |
| | | | XI-1:: SrPyrH | |
| ST10071 | ST9336 | pCfB9712(XII- | MATa URA3 | This |
| | | 4MF: | HIS3 LEU2 | study |
| | | LaRebH), | TRP1 MAL2-8c | |
| | | | SUC2 XI-3:: | |
| | | | CrTdc XII-4:: | |
| | | | LaRebH | |
| ST10073 | ST9766 | pCfB9712(XII- | MATa URA3 | This |
| | | 4MF: | HIS3 LEU2 | study |
| | | LaRebH), | TRP1 MAL2-8c | |
| | | | SUC2 XI-3:: | |
| | | | CrTdc XII-5:: | |
| | | | Stth-LaRebF | |
| | | | XII-4:: LaRebH | |
| ST9740 | ST9328 | BB4320 (X- | MATa Δura3 | This |
| | | 4MF: PcCYB5 | HIS3 LEU2 | study |
| | | | TRP1 MAL2-8c | |
| | | | SUC2 XII-5: | |
| | | | PcPsiM- | |
| | | | PcPsiK XI-3:: | |
| | | | CrTdc-PcPsiH | |
| | | | XI-1:: pTEF1−> | |
| | | | PcCpr X-2:: | |
| | | | URA3 X-4:: | |
| | | | PcCYB5 | |
| ST10290 | ST7574 | pCfB9713(XII- | MATa URA3 | This |
| | | 4MF: | HIS3 LEU2 | study |
| | | LaRebH- | TRP1 MAL2-8c | |
| | | LaRebF) | SUC2 XII- | |
| | | | 4: LaRebH- | |
| | | | LaRebF | |

Plasmid and Strain Construction

E. coli DH5 was used for all plasmid cloning and propagation. Single integration plasmids were constructed using the EasyClone-MarkerFree system (Jessop-Fabre et al., 2016), and multiple integration plasmids were constructed using a modified version of the EasyCloneMulti system (Maury et al., 2016) using a backbone plasmid were multiple integration was achieved using a Kluyveromyces lactis URA3 gene (KIURA3) under control of a truncated 10 bp KIURA3 promoter. Heterologous genes were codon-optimized for expression in S. cerevisiae using the JCat algorithm (Grote et al., 2005) and ordered as synthetic gene strings (GeneArt). DNA was transformed into S. cerevisiae using the LiAc method according to (Gietz and Woods, 2002).

Cultivation and Analysis

E. coli cells were cultured at 37° C. with shaking at 300 rpm. S. cerevisiae cells were cultured at 30° C. with shaking at 300 rpm. For micro-titer plate (MTP) cultivation of psilocybin producing S. cerevisiae strains, cells were inoculated from a 400 μL synthetic media pre-culture into 500 μL synthetic FIT media in a 96-deep well microtiter plate with air-penetrable lid (EnzyScreen, NL) and incubated for 72 h. When required, uracil was added at a final concentration of 200 mg/L.

Extraction of extracellular metabolites for analysis was performed as follows; Cell culture broth was supplemented with 100% acetonitrile at a ratio of 1:1, vortexed thoroughly then centrifuged at 3000 g for 5 min. The resulting supernatant was further diluted in 50% acetonitrile if required and analyzed using LC-MS with the following conditions; High resolution LC-MS measurements were carried out on a Dionex UltiMate 3000 UHPLC, connected to an Orbitrap Fusion Mass Spectrometer. The UHPLC was equipped with a zic-Hilic column, 15 cm×2.1 mm, 3 μm column. The temperature was 35.0 and the flow rate 0.5 mL/min. The system was running an isocratic gradient with a mobile phase consisting of 20% 10 mM ammonium formate (pH 3) and 80% acetonitrile, with 0.1% formic acid. The samples were passed on to the MS equipped with a heated electrospray ionization source (HESI) in positive-ion mode. The scan range was 100 to 1000 Da. Psilocybin, psilocin, tryptophan and tryptamine authentic analytical standards were used to quantify production in engineered strains.

### Example 2: Expression of a Heterologous Psilocybin Biosynthetic Pathway in S. cerevisiae

Heterologous genes encoding the catalytic enzymes for psilocybin biosynthesis were introduced into S. cerevisiae strain ST8251 (CEN.PK113-5D+Cas9) (as outlined in FIG. 2). The biosynthetic production of psilocybin starts with L-tryptophan, which is converted into tryptamine by Tryptophan decarboxylase. Catharanthus roseus (C. roseus) Tryptophan decarboxylase (CrTdc) was used (Brown et al., 2015). Tryptamine is next converted into 4-Hydroxytryptamine by a cytochrome P450 containing monooxygenase (PcPsiH). Cytochrome P450 enzymes are characterized by their dependency on a cytochrome P450 reductase (CPR) which facilitates electron transfer between NADPH and cytochrome P450 enzymes (Renault et al., 2014). In S. cerevisiae, this is encoded by NCP1. 4-Hydroxytryptamine is next converted into norbaeocystin by a 4-Hydroxytryptamine kinase encoded by PcPsiK. Finally, an N-methyltransferase encoded by PcPsiM mediates the iterative methyl transfer of norbaeocystin to baeocystin then to psilocybin.

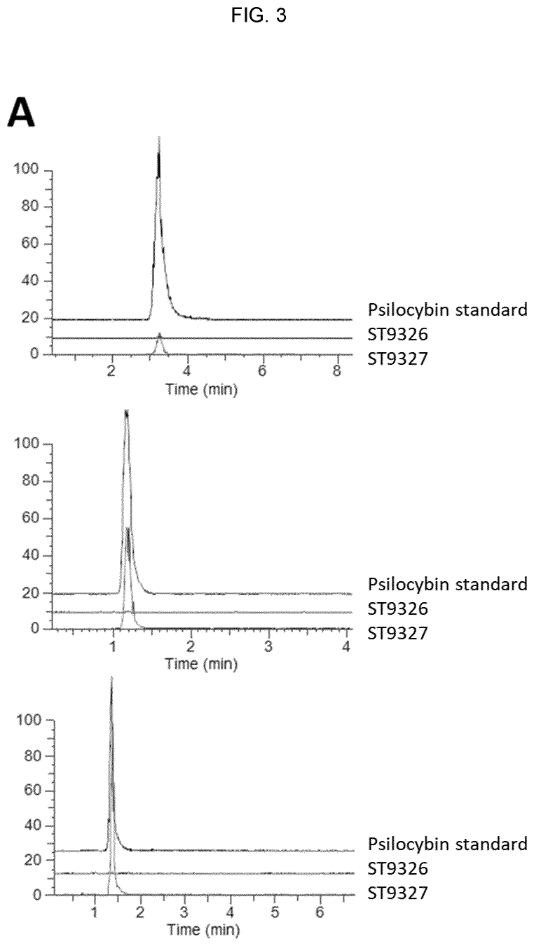

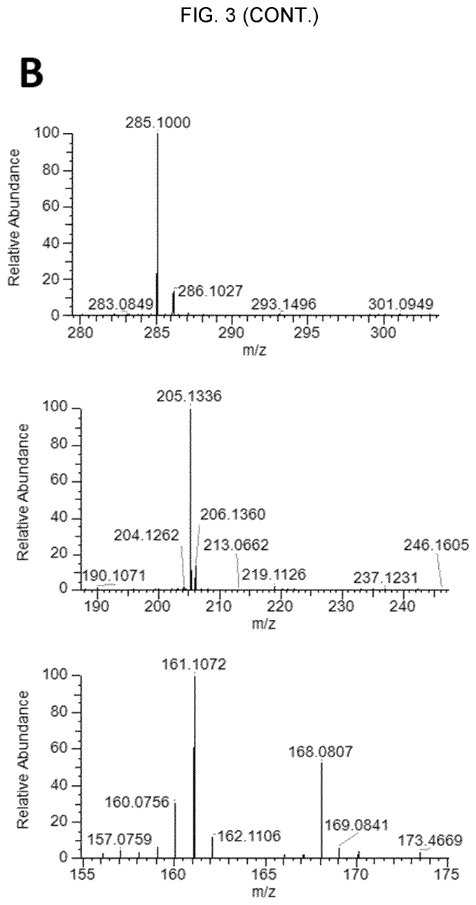

The basic heterologous pathway was introduced into S. cerevisiae (ST9327), then, using an Orbitrap Fusion Mass Spectrometer and authentic analytical standards, successful production of psilocybin, as well as the pathway intermediate tryptamine, and the spontaneous degradation product psilocin was confirmed in micro-titer plate cultivation (FIG. 3). Quantification using a calibration curve determined that ST9327 produced tryptamine, psilocybin and psilocin with titers of 90 mg/L 25 mg/L and 44 mg/L, respectively, in synthetic FIT media.

### Example 3: Expression of a Novel Cytochrome P450 Reductase (CPR) from P. cubensis Increased Psilocybin Production

While psilocybin was successfully produced in yeast, the initial titers were low. Furthermore, analysis revealed the extracellular accumulation of tryptamine (90 mg/L) indicating a limitation in the conversion of tryptamine to 4-Hydroxytryptamine encoded by the cytochrome P450 enzyme PcPsiH (FIG. 3A). Cytochrome P450 enzymes (CYP) belong to a superfamily of heme-containing monooxygenases and require a cytochrome P450 reductase (CPR) partner to deliver one or more electrons to reduce the heme-bound iron and oxidized substrates (Renault et al., 2014). While the detection of psilocybin indicated that the native S. cerevisiae CPR (Ncp1) could carry out this reduction, the accumulation of tryptamine suggested a sub-optimal interaction between the two enzymes. We tested the function of a putative P. cubensis CPR (PcCpr) with 42.2% homology to the Ncp1 amino acid sequence by expressing it from the strong constitutive promoter TEF1 and the medium-strength constitutive promoter TEF2. An additional CPR from Arabidopsis thaliana (A. thaliana) AtAtr2 was also tested with additional overexpression of the S. cerevisiae cytochrome b5 (CYB5) as this has previously been shown to enable functional expression of other CYP's (Li et al., 2016). While introduction of AtAtr2 and PcCpr from a medium-strength TEF2 promoter resulted in a decrease in titer, expression of PcCpr from the strong TEF1 promoter produced a significant increase in psilocybin and psilocin titer, reaching 117 mg/L and 135 mg/L respectively (FIG. 3A). This not only confirmed the functional expression of a novel CPR from P. cubensis, but also highlighted the importance of investigating the compatibility and expression of a CYP with a suitable CPR partner.

### Example 4: Metabolic Engineering of Tryptophan Precursor Supply Results in High Psilocybin Titers

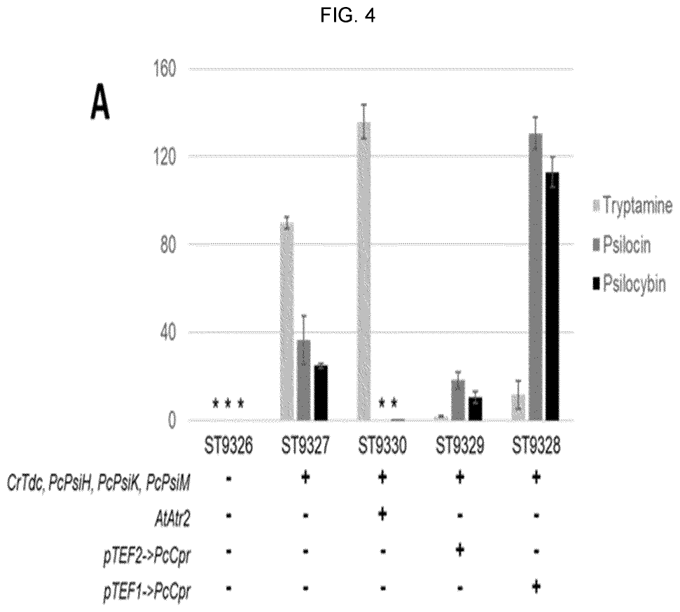

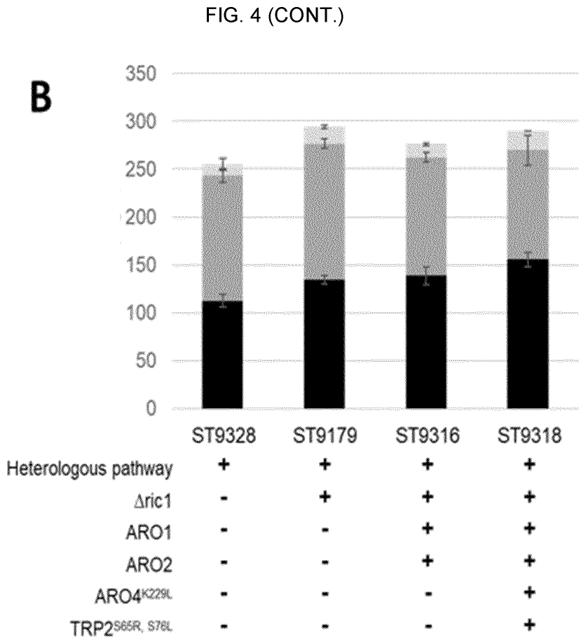

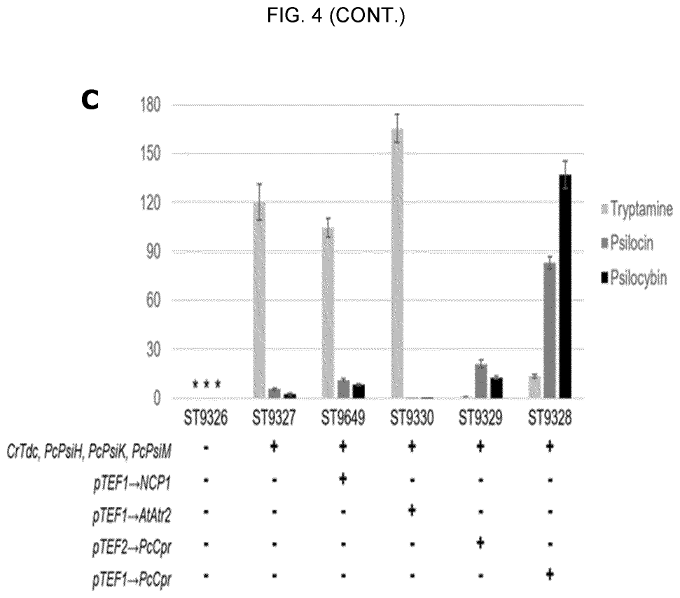

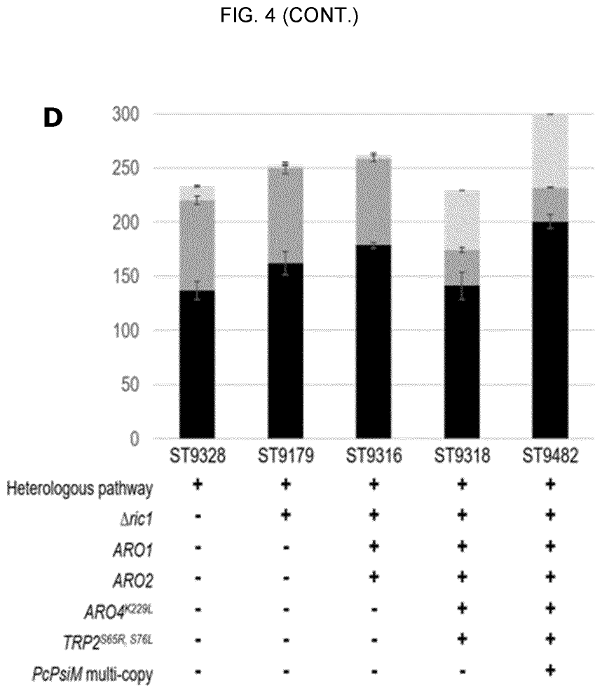

Functional implementation of PcCpr resulted in a significant increase in psilocybin and psilocin titers, and furthermore, significantly reduced extracellular accumulation of the first product in the heterologous pathway, tryptamine (11.7 mg/L, FIG. 4A). Overall, this suggested that the heterologous pathway had sufficient capacity to convert available tryptophan to psilocybin and that we should next focus our efforts on boosting the native precursor supply.

To boost psilocybin precursor supply we introduced a series of modifications including overexpression of genes in the shikimate pathway (ARO1, ARO2), overexpression of feedback insensitive mutant genes in the shikimate pathway (ARO4^{K229L}, TRP2^{S65R, S76L}) (Graf et al., 1993; Luttik et al., 2008), and deletion of genes involved in regulation of the shikimate pathway (RIC1) (Suástegui et al., 2017). Iterative introduction of these modifications led to a modest yet significant increase in psilocybin and psilocin titer with ST9318 producing 155 mg/L psilocybin and 113 mg/L psilocin (FIG. 4B).

### Example 5: Production of Tryptamine Derivatives

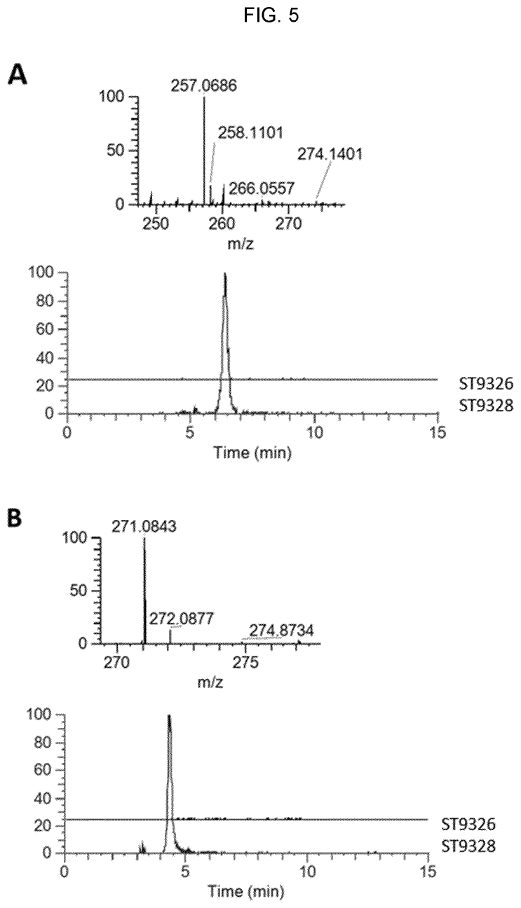

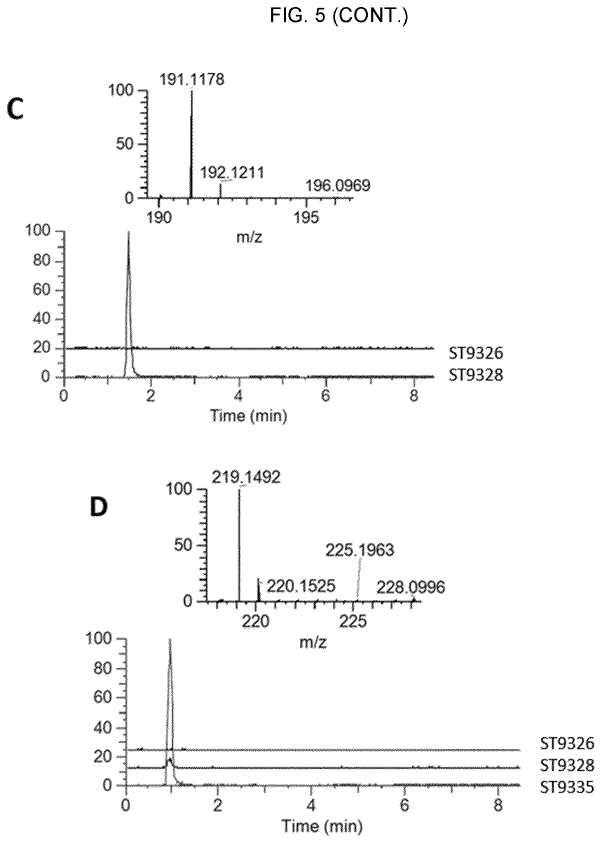

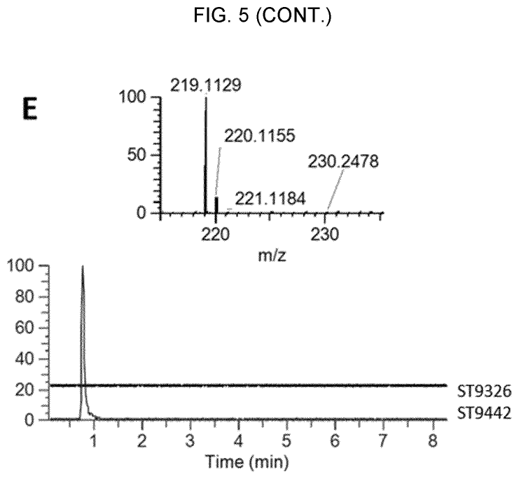

Finally, in an attempt to expand the utility of these engineered strains, we investigated whether S. cerevisiae could be engineered to produce natural and new-to-nature tryptamine derivatives. While enzymes typically display strict substrate specificity, others display relaxed substrate specificity and can accept multiple substrates with various affinities. LC-MS analysis of the psilocybin producing strain ST9328 detected the presence of norbaeocystin (non N-methylated) (FIG. 5A), baeocystin (mono-N-methylated) (FIG. 5B) and psilocybin (di-N-methylated), as well as their dephosphorylated derivatives psilocin, and norpsilocin (dephosphorylated baeocystin) (FIG. 5C) catalyzed by PcPsiM.

We then investigated whether this enzyme could catalyze a third iterative N-methylation to produce the tri-methylated derivative aeruginascin (Jensen et al., 2006). Interestingly, while we could not observe a peak matching the expected m/z of aeruginascin, a peak matching the expected m/z of the dephosphorylated version of aeruginascin was detected in ST9328. Furthermore, introduction of multiple copies of PcpsiM using a Ty integrative vector (ST9335) led to a 10-fold increase in the dephosphorylated aeruginascin peak area (FIG. 5D) and a corresponding decrease in the norbaeocystin and baeocystin peak areas, thereby strongly corroborating the production of the tri-N-methylated derivative. Finally, to demonstrate the production of new-to-nature derivatives, we investigated whether serotonin-N-acetyl transferase from Bos taurus (BtAANAT), previously demonstrated to convert 5-hydroxy tryptamine (serotonin) into N-acetyl serotonin (Normelatonin) in S. cerevisiae (Germann et al., 2016) could also accept 4-hydroxy tryptamine. Detection of a peak matching the expected m/z of N-acetyl-4-hydroxy tryptamine in ST9442 demonstrated not only the relaxed substrate specificity of BtAANAT, but also the successful production of a novel molecule structurally similar to both psilocin and normelatonin with potentially novel pharmacological activity (FIG. 5E).

### Example 6: Psilocybe cubensis Cytochrome b5 Expression Improves Psilocybin Titers

To investigate whether introduction of a P. cubensis cytochrome b5 could improve psilocybin titers, efforts to identify a cytochrome b5-encoding gene in the P. cubensis genome were undertaken. A gene encoding a putative P. cubensis cytochrome b5 (PcCYB5) was identified. To test its function and effect on psilocybin titers, PcCYB5 was expressed in ST9328 which carries the fully psilocybin biosynthetic pathway plus the P. cubensis cytochrome P450 reductase (PcCPR), resulting in strain ST9740. Strains were cultivated in media containing 5 g/L tryptophan in order to drive as much flux as possible through the pathway in order to distinguish whether expression of PcCYB5 could facilitate a higher conversion of tryptamine to 4-hydroxytryptamine.

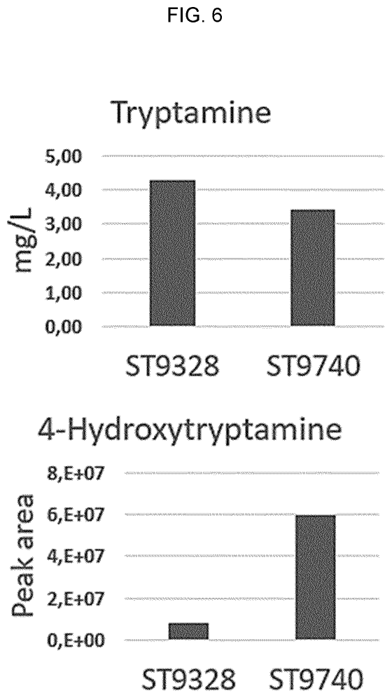

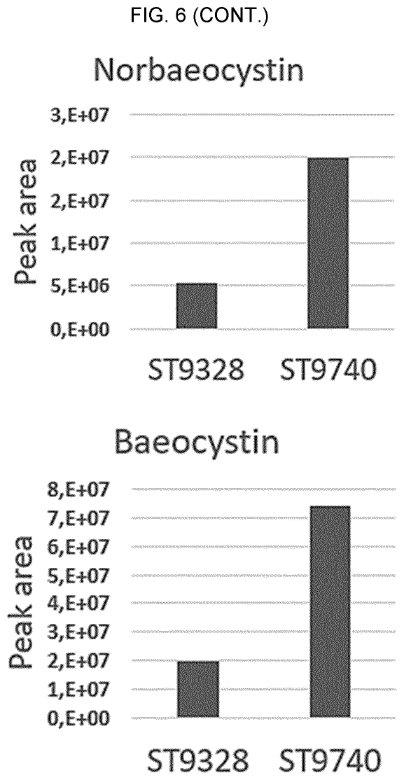

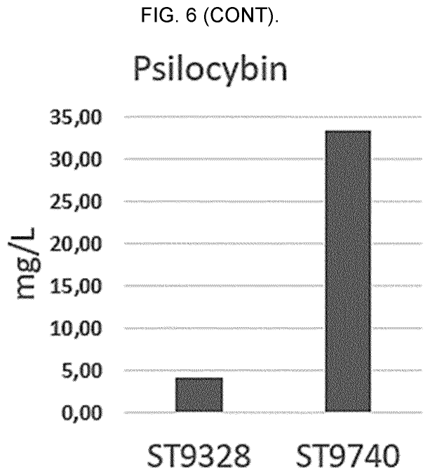

LC-MS analysis (FIG. 6) shows that PcCYB5 expression results in a significant increase in production of psilocybin and all pathway intermediates after the tryptamine hydroxylation step. These data suggest that PcCYB5 has a significant positive impact on the conversion of tryptamine to 4-hydroxytryptamine.

### Example 7: ERG4 and SPE2 Knock Out Increases Psilocybin Production in Yeast

We speculated that N-methylation catalyzed by PcPsiM was a rate-limiting step in the psilocybin pathway, perhaps due to insufficient availability of SAM, which acts as the methyl donor in the reaction. Accordingly, we set out to investigate strategies for increasing the availability of SAM in S. cerevisiae.

Hence, we performed single gene knock-outs of SPE2 and ERG4, respectively, in the high psilocybin producing strain ST9316, yielding strains ST9733 and ST9734.

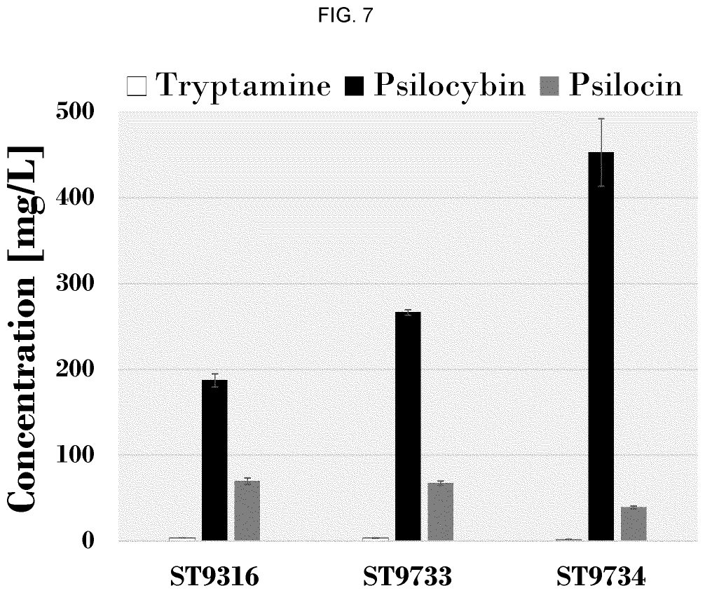

According to LC-MS analysis, both ST9733 and ST9734 produced higher titers of psilocybin compared to the control ST9316 (FIG. 7). ERG4 knock-out had the highest effect and resulted in a two-fold increase of psilocybin titers compared to the control. T-tests found the increases in psilocybin titers of both knock-outs significant (SPE2 p=0.023 and ERG4 p=0.033). Hence, both investigated knock-outs had a positive effect on psilocybin titers. These results implicate that part of the reason why N-methylation is a rate-limiting step in the psilocybin biosynthesis can be explained by SAM availability.

### Example 8: POS5 Overexpression Increases Psilocybin Production in Yeast

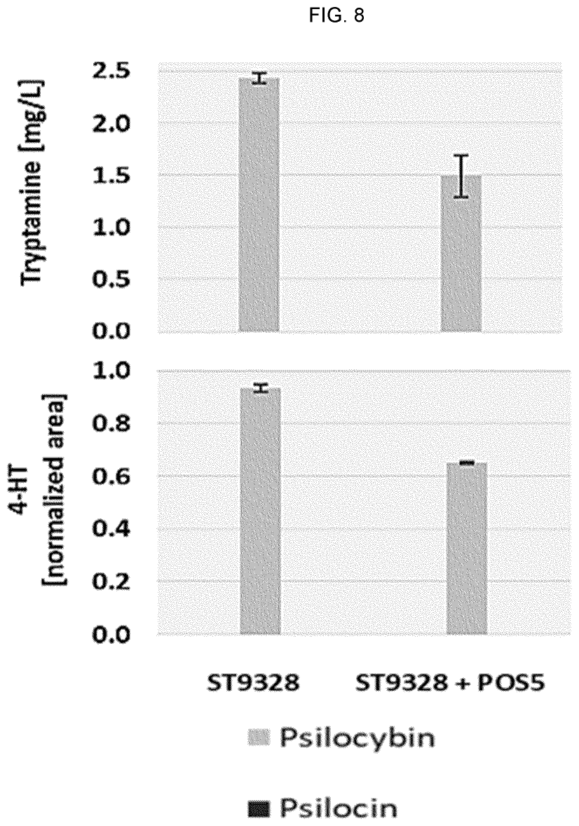

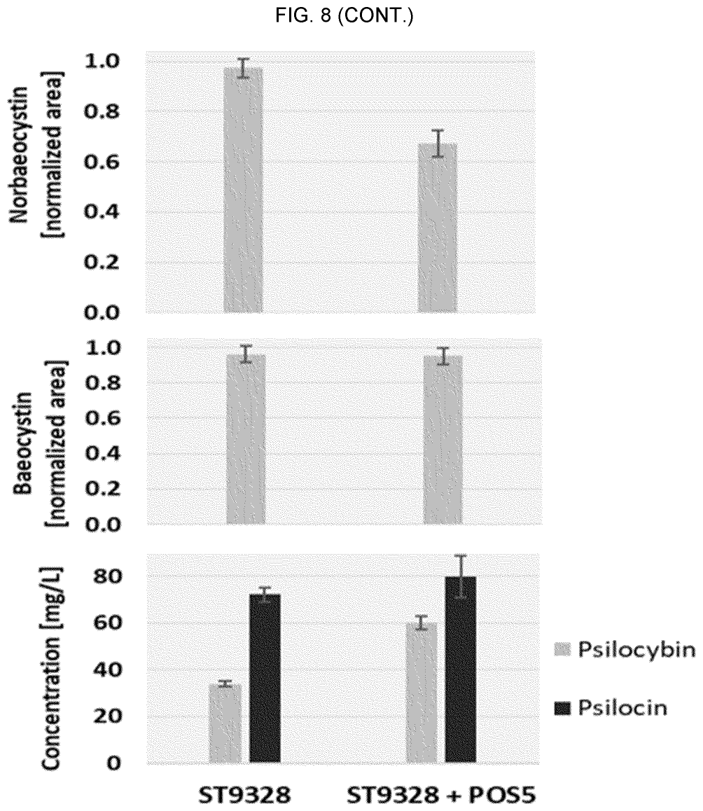

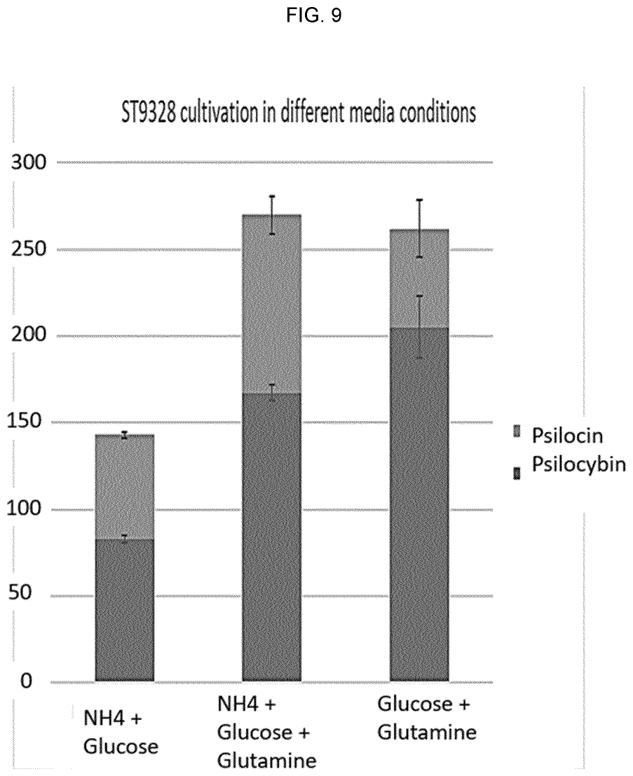

The 4-hydroxylation step in the psilocybin biosynthetic pathway where tryptamine is converted to 4-HT by PcPsiH consumes NADPH. We hypothesized that the supply of NADPH might be a limiting factor for this catalytic reaction, hence we explored different strategies in attempts to increase NADPH supply and thereby psilocybin production, namely overexpression of the native S. cerevisiae gene POS5 in the psilocybin producing strain ST9328. POS5 encodes a mitochondrial NADH kinase, responsible for NADPH generation in the mitochondria. Overexpression of POS5 in ST9328 led to a two-fold increase in psilocybin titers (FIG. 8), potentially due to increased NADPH supply to the PcPsiH catalyzed reaction.

### Example 9: Glutamine Supplementation Increases Psilocybin Titers

All tryptamine derivatives including psilocybin are produced from the common intermediate tryptophan which itself is produced from the amino acids serine and glutamine. While metabolic engineering to increase flux through the shikimate pathway (e.g. by overexpression of ARO1 and ARO2) had a positive effect on psilocybin titers, boosting the shikimate pathway flux only considers the carbon skeleton of tryptophan and not the two nitrogen groups present on the molecule. We therefore hypothesized that increasing flux towards the nitrogen groups of tryptophan would have a positive effect. To test this, ST9328 was cultivated in different media containing 5 g/L glutamine. LC-MS analysis showed that strains cultivated in glutamine containing media produced significantly higher amounts of psilocybin and psilocin with a 2-fold increase in titer observed.

### Example 10: Production of Methylated Tryptamine Derivatives

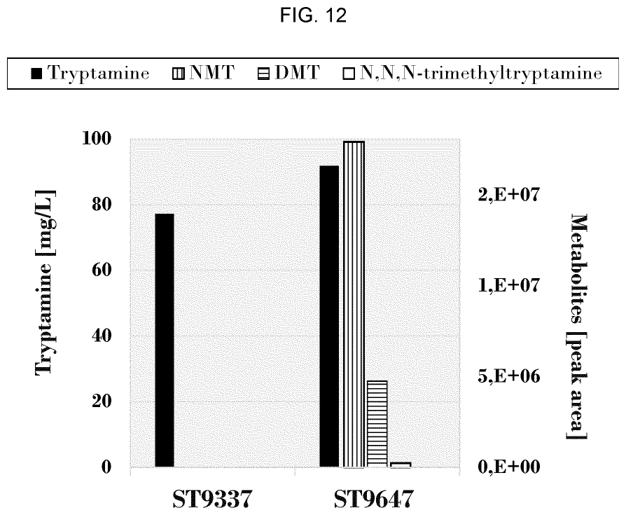

Human and rabbit (Oryctolagus cuniculus) INMTs have both been cloned and heterologously expressed in COS-1 cells (immortalized Chlorocebus aethiops kidney cells). The O. cuniculus derived INMT (OcINMT) showed higher affinity in vitro for tryptamine than the human derived INMT (Km of 0.27 and 2.92, respectively), and was selected for production of DMT. Multiple copies of the OcINMT gene were integrated into the genome of strain ST9337, which expresses CrTDC and produces approximately 70 mg/L tryptamine. This yielded strain ST9647 that when cultivated produced metabolites with masses and fragmentation patterns corresponding to N-methyltryptamine (NMT), N,N-dimethyltryptamine (DMT) and N,N,N-trimethyltryptamine (TMT) as shown in FIG. 12.

### Example 11: Production of Halogenated Tryptophan and Halogenated Tryptophan Derivatives

In nature, halogenated compounds are produced by haloperoxidases, perhydrolases and flavin-dependent halogenases. While haloperoxidases and perhydrolases in general lack regioselectivity, flavin-dependent halogenases display a high degree of substrate specificity and regioselectivity, which could make them more amenable for applications in biotechnological production. In addition to oxygen and halide ions, flavin-dependent halogenases require input of FADH₂ provided by a partner flavin-reductase that performs NADH-driven reduction of FAD to FADH₂.

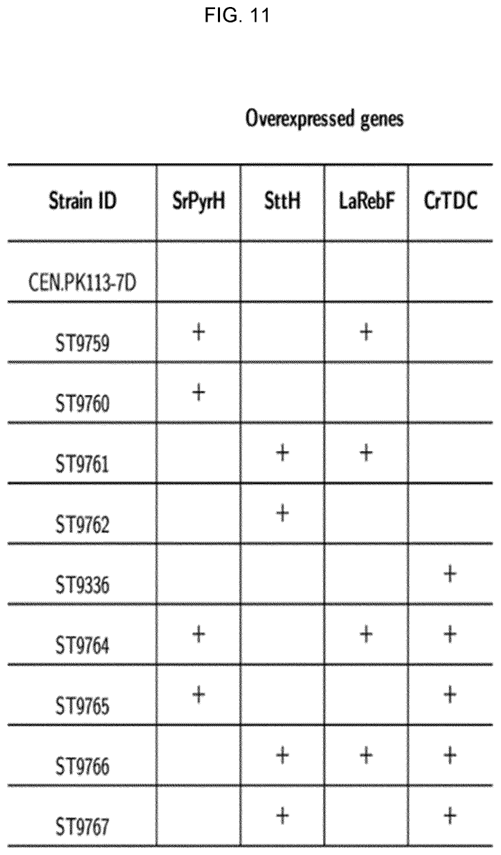

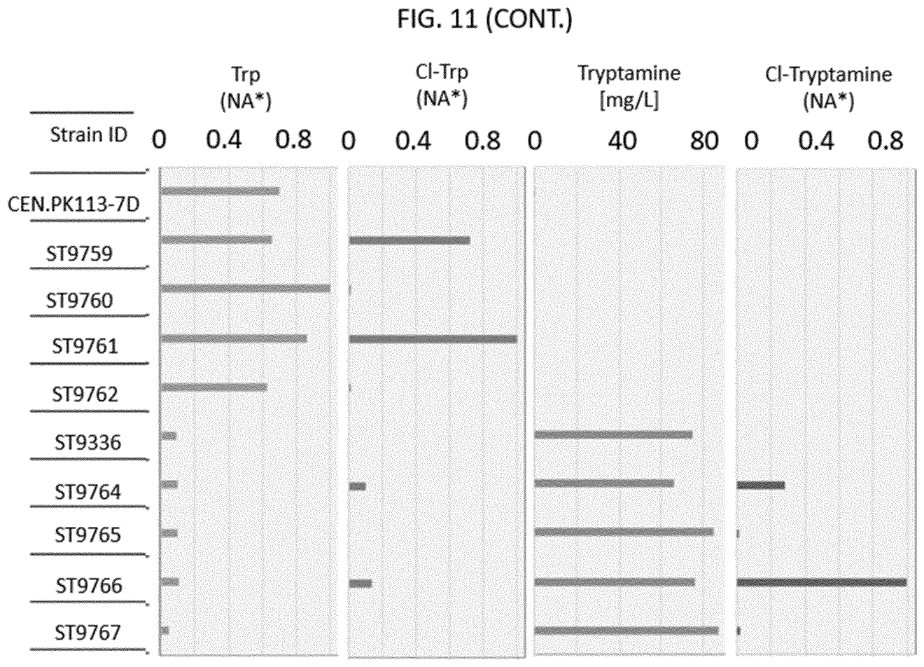

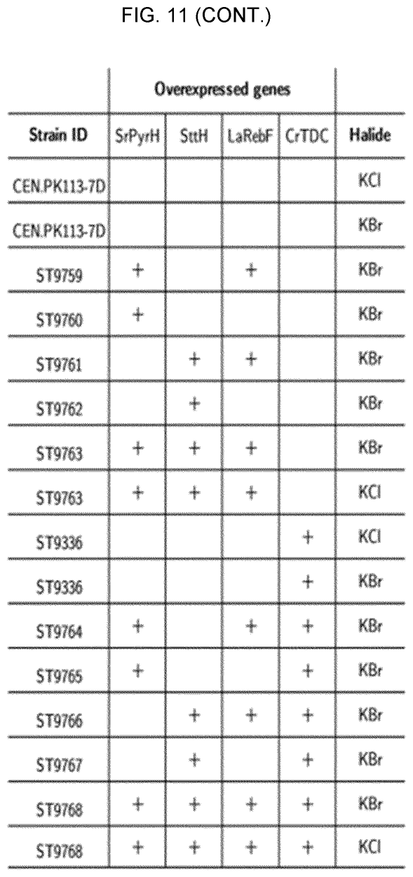

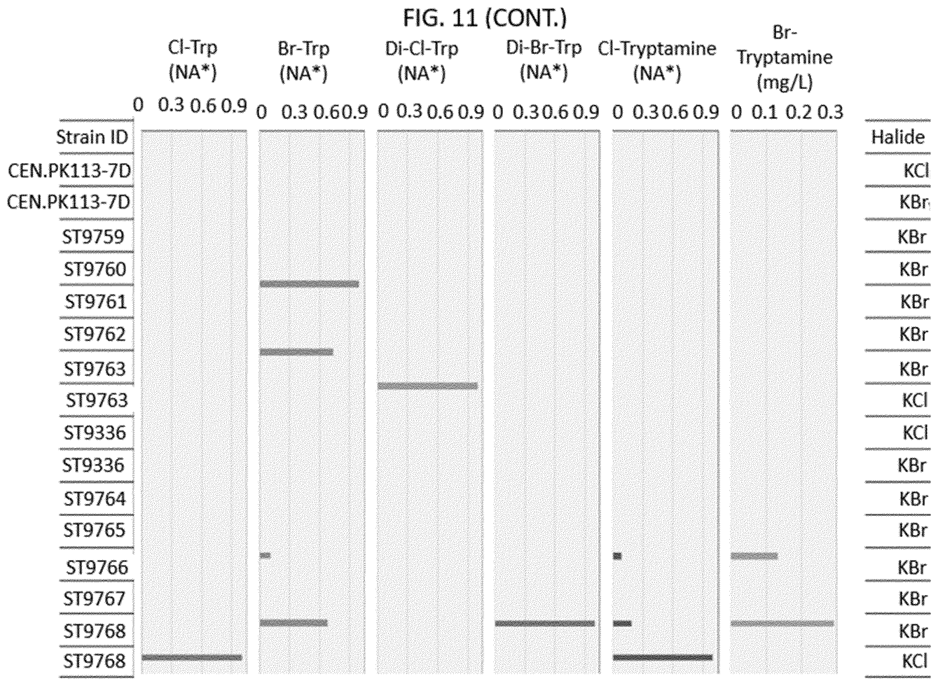

To attempt production of halogenated tryptophan, integrations of Tryptophan 5-halogenase (SrPyrH), tryptophan 6-halogenase (SttH) and tryptophan 7-halogenase (LaRebH) into a wildtype S. cerevisiae strain (ST7574) were carried out with and without co-integration of the partner flavin reductase LaRebF. The same integrations were additionally performed in strain ST9336 expressing CrTDC in order to attempt production of halogenated tryptamine. Transformants were cultivated in synthetic minimal media supplemented with 25 mM KCl or 25 mM KBr. For extraction of intracellular products, the cultivation broths were subjected to cell lysis, which was carried out by adding a small aliquot of acid washed glass beads (212-300 p, Sigma) and running the samples for two cycles of 20 sec. at 5500 rpm on a Precellys 24 Homogenizer. The lysed cell broths were centrifuged at 17000 g for 1 min. and the supernatants were analyzed by LC-MS. Chloro- and bromo-tryptophan were present in all wild-type (ST7574) strains expressing a tryptophan halogenase, even in absence of LaRebF (FIG. 11).

This observation was surprising. Flavin-dependent halogenases are speculated to use free FADH₂ supplied by the flavin reductase, rather than forming a complex with the flavin reductase. FADH₂ is produced in the citric acid cycle in S. cerevisiae, but as this takes place in the mitochondria it is unlikely that the FADH₂ generated this way could be responsible for the observed halogenation. However, the observed chlorination and bromination in absence of LaRebF indicates that FADH₂ was present in the cytosol of S. cerevisiae. However, the levels of halogenated tryptophan were approximately 100-fold higher in the presence of LaRebF, clearly demonstrating that FADH₂ was a limiting factor of the chlorination and that expression of LaRebF increases cytosolic FADH₂. Chloro- and bromotryptamine was produced by strains expressing CrTDC and a halogenase. Since none of these halogenases have been shown to chlorinate tryptamine, this observation indicates that CrTDC expressed in S. cerevisiae can accept chlorinated and brominated tryptophan as a substrate. In order to determine the efficiency of production of these halogenated derivatives the production of bromotryptamine was compared with an authentic analytical standard of 5-bromotryptamine. Comparison revealed that these strains produced approximately 0.13-0.29 mg/L of bromotryptamine.

By expressing different combinations of halogenases in a single strain, the production of di-halo and di-bromo tryptophan was observed (FIG. 11 and table 2). This di-halogenation was not observed in strains containing only a single halogenase thereby highlighting the specificity and regioselectivity of these reactions.

TABLE 2
Halogenated metabolite production in recombinant yeast strains expressing
tryptophan-7-halogenase (LaRebH) and/or tryptophan-6-halogenase (Stth). The
halogenases we’re expressed in the presence and absence of the partner flavin
reductase (LaRebF) as well as in the presence and absence of the tryptophan
decarboxylase (CrTdc) as indicated. “+” denotes that a single copy of the relevant gene
was integrated into the genome. Strains were cultivated in synthetic minimal media
supplemented with 25 mM KCl or 25 mM KBr. “Halo” refers to either the chlorinated or
brominated derivative as indicated in the “Halide” column. Metabolites from cultivated
strains were extracted using the intracellular extraction protocol and analysed by LC-
MS. Numbers shown are total peak area for peaks which match the m/z and
fragmentation pattern of the metabolite of interest.

| | Overexpressed genes | | | | | |
|---|---|---|---|---|---|---|
| Strain ID | SrPyrH | SttH | | LaRebH | LaRebF | CrTDC |
| ST9336 | | | | | | + |
| ST10071 | | | | + | | + |
| ST10073 | | + | | + | | + |
| ST9336 | | | | | | + |
| ST10071 | | | | + | | + |
| ST10073 | | + | | + | | + |
| | | Halo- | Di-halo- | | Halo- | Di-halo- |
| Strain ID | Tryptophan | tryptophan | tryptophan | Tryptamine | tryptamine | tryptamine |
| ST9336 | 939885.22 | BDL | BDL | 5608790070.22 | BDL | BDL |
| ST10071 | 6470046.51 | 245987.36 | BDL | 5380588439.39 | 1060557.93 | BDL |
| ST10073 | 4670708.93 | 248062.49 | 8860152.30 | 4094516146.00 | 8311942.68 | BDL |
| ST9336 | 734583.32 | BDL | BDL | 6846137.38 | BDL | BDL |
| ST10071 | 5791165.29 | 421434.39 | BDL | 5966694940.37 | 344755.15 | BDL |
| ST10073 | 4117885.98 | 4749158.73 | 6979889.05 | 4909694886.11 | 3085737.98 | BDL |

BDL: Below Detection Limit.

### Example 12: Direct Halogenation of Tryptamine by Tryptophan Halogenases

In order to evaluate the substrate specificity of tryptophan halogenases, their ability to directly halogenate a tryptophan derivative like tryptamine was tested. Strains expressing individual tryptophan halogenases together with the partner flavin reductase LaRebF were cultivated in synthetic minimal media supplemented with 25 mM KCl or 25 mM KBr and 1 mM tryptamine. Cultivation samples were subjected to the intracellular extraction method and the supernatants were analyzed by LC-MS. The production of halogenated tryptamine was observed in strains expressing SttH and LaRebH when tryptamine was supplemented to the media (Table 3).

TABLE 3
Direct halogenation of tryptamine in yeast strains expressing tryptophan
halogenases. The tryptophan halogenases SrPyrH (tryptophan-5-halogenase), SttH
(tryptophan-6-halogenase), and LaRebH (tryptophan-7-halogenase) were expressed in
the presence of the partner flavin reductase (LaRebF). “+” denotes that a single copy of
the relevant gene was integrated into the genome. Strains were cultivated in synthetic
minimal media supplemented with 25 mM KCl or 25 mM KBr and with or without 1 mM
tryptamine, as indicated with “+” or “−”, respectively. Metabolites from cultivated strains
were extracted using the intracellular extraction protocol and analyzed by LC-MS.
Numbers shown are total peak area for peaks which match the m/z and fragmentation
pattern of the metabolite of interest.

| | | | | | Medium | |
|---|---|---|---|---|---|---|
| | Overexpressed genes | | | | supplementation | |
| Strain ID | SrPyrH | SttH | LaRebH | LaRebF | Halide | Tryptamine |
| CEN.PK113-7D | − | − | − | − | KCl | − |
| ST9759 | + | − | − | + | KCl | − |
| ST9761 | − | + | − | + | KCl | − |
| ST10290 | − | − | + | + | KCl | − |
| CEN.PK113-7D | − | − | − | − | KCl | + |
| ST9759 | + | − | − | + | KCl | + |
| ST9761 | − | + | − | + | KCl | + |
| ST10290 | − | − | + | + | KCl | + |
| CEN.PK113-7D | − | − | − | − | KBr | − |
| ST9759 | + | − | − | + | KBr | − |
| ST9761 | − | + | − | + | KBr | − |
| ST10290 | − | − | + | + | KBr | − |
| CEN.PK113-7D | − | − | − | − | KBr | + |
| ST9759 | + | − | − | + | KBr | + |
| ST9761 | − | + | − | + | KBr | + |
| ST10290 | − | − | + | + | KBr | + |

| | Detected metabolites | | |
|---|---|---|---|
| Strain ID | Tryptamine | Chlorotryptamine | Bromotryptamine |
| CEN.PK113-7D | BDL | BDL | BDL |
| ST9759 | BDL | BDL | BDL |
| ST9761 | BDL | BDL | BDL |
| ST10290 | BDL | BDL | BDL |
| CEN.PK113-7D | 7079447233.68 | BDL | BDL |
| ST9759 | 7267166632.10 | BDL | BDL |
| ST9761 | 7420512884.33 | 476979.11 | BDL |
| ST10290 | 6222814954.76 | BDL | BDL |
| CEN.PK113-7D | BDL | BDL | BDL |
| ST9759 | BDL | BDL | BDL |
| ST9761 | BDL | BDL | BDL |
| ST10290 | BDL | BDL | BDL |
| CEN.PK113-7D | 3539164951.56 | BDL | BDL |
| ST9759 | 3671688293.50 | BDL | BDL |
| ST9761 | 3944708049.35 | 201736.99 | 156386.87 |
| ST10290 | 3427950061.07 | BDL | 49322.63 |

BDL: Below Detection Limit.

### Example 13: Engineering Saccharomyces boulardii for Advanced Microbiome Therapeutics

Psilocybin has been found to bind various receptors of serotonin, an important neurotransmitter relevant to various psychological and neurological afflictions, e.g. depression and migraine. Serotonin receptors are widely expressed in the gut, in fact it has been estimated that around 95% of serotonin receptors are found in the GI tract. It is becoming increasingly clear that the neural networks of the brain (central) and gut (enteric) are tightly linked, and that the bidirectional regulatory signals exchanged between these organs can be a significant contributor or mediator of disease. The complexities of the brain-gut connection have yet to be fully unraveled, however the recent recognition of the importance of the gut microbiome to the physiological function of the gut and brain, confirms the possibility of manipulating neurological function via the gut. This presents an intriguing opportunity to engineer probiotic organisms, e.g. Saccharomyces boulardii for the in vivo delivery of various beneficial compounds. Auxotrophic S. boulardii strains (ΔURA3 ΔHIS3 ΔTRP1 ΔLEU2) are generated from Saccharomyces cerevisiae Meyen ex E. C. Hansen (ATCC MYA-796) by introducing a stop codon near the beginning of the genes, thereby disrupting their expression. The Cas9 plasmid (pCfB2312) is introduced using the LiAc method according to (Gietz and Woods, 2002), and maintained on 200 mg/L G418. Using the EasyClone-MarkerFree system, integration plasmids containing SEQ ID NO: 6-7, 8, and 9-10 are constructed and integrated into the S. boulardii genome at the X-3, X-4, and XII-5 integration sites, respectively. The gRNA is maintained via URA auxotrophy or nourseothricin sensitivity (50 mg/L; S. boulardii exhibits heightened nourseothricin sensitivity compared to S. cerevisiae). The production of psilocybin-related tryptamine derivatives is achieved via the introduction of any SEQ ID NO: 6-31; 37-41; 43; 45; 47; 49; 55-59 DNA sequences, or other sequences encoding the necessary enzymes. In order to increase production under GI conditions, the yeast can be subjected to various optimisations, e.g. medium optimization (e.g. growth on alternative carbon sources) or by modification of the biosynthesis pathway (e.g. introducing, reducing or modifying alternative anaerobic biosynthesis steps).

Sequences

| SEQ | | Organism and optionally accession |
|---|---|---|
| ID NO: | Description | number |
| 1 | CrTDC | Catharanthus roseus, |
| | (protein) | ACCESSION AYA72254 |
| 2 | PcPsiH | Psilocybe cubensis, |
| | (protein) | ACCESSION ASU62246 |
| 3 | PcCpr | Psilocybe cubensis |
| | (protein) | |
| 4 | PcPsiK | Psilocybe cubensis, |
| | (protein) | ACCESSION ASU62237 |
| 5 | PcPsiM | Psilocybe cubensis, |
| | (protein) | ACCESSION ASU62238 |
| 6 | CrTDC | Catharanthus roseus |
| | (DNA) | |
| 7 | PcPsiH | Psilocybe cubensis |
| | (DNA) | |
| 8 | PcCpr | Psilocybe cubensis |
| | (DNA) | |
| 9 | PcPsiK | Psilocybe cubensis |
| | (DNA) | |
| 10 | PcPsiM | Psilocybe cubensis |
| | (DNA) | |
| 11 | BtAANAT | Bos taurus |
| | protein | |
| 12 | ARO4 | Saccharomyces cerevisiae |
| | (DNA) | P32449 |
| 13 | ARO1 | Saccharomyces cerevisiae |
| | (DNA) | P08566 |
| 14 | ARO2 | Saccharomyces cerevisiae |
| | (DNA) | P28777 |
| 15 | TRP1 | Saccharomyces cerevisiae |
| | (DNA) | P00912 |
| 16 | TRP2 | Saccharomyces cerevisiae |
| | (DNA) | P00899 |
| 17 | TRP3 | Saccharomyces cerevisiae |
| | (DNA) | P00937 |
| 18 | TRP4 | Saccharomyces cerevisiae |
| | (DNA) | P07285 |
| 19 | TRP5 | Saccharomyces cerevisiae |
| | (DNA) | P00931 |
| 20 | SER1 | Saccharomyces cerevisiae |
| | (DNA) | P33330 |
| 21 | SER2 | Saccharomyces cerevisiae |
| | (DNA) | P42941 |
| 22 | SER3 | Saccharomyces cerevisiae |
| | (DNA) | P40054 |
| 23 | ARO8 | Saccharomyces cerevisiae |
| | (DNA) | P53090 |
| 24 | ARO9 | Saccharomyces cerevisiae |
| | (DNA) | P38840 |
| 25 | GLT1 | Saccharomyces cerevisiae |
| | (DNA) | Q12680 |
| 26 | CDC19 | Saccharomyces cerevisiae |
| | (DNA) | P00549 |
| 27 | STB5 | Saccharomyces cerevisiae |
| | (DNA) | P38699 |
| 28 | POS5 | Saccharomyces cerevisiae |
| | (DNA) | Q06892 |
| 29 | ZWF1 | Saccharomyces cerevisiae |
| | (DNA) | P11412 |
| 30 | BtAANAT | Bos taurus |
| | (DNA) | |
| 31 | SER33 | Saccharomyces cerevisiae |
| | (DNA) | P40510 |
| 32 | SrPyrH | Streptomyces rugosporus tryptophan-5- |
| | (protein) | halogenase 1.14.19.58 (Uniprot ID: A4D0H5) |
| 33 | SttH | Streptomyces toxytricini tryptophan 6- |
| | (protein) | halogenase EC 1.14.19.59 (Uniprot ID: |
| | | E9P162) |
| 34 | LaRebH | Lechevalieria aerocolonigenes Tryptophan 7- |
| | (protein) | halogenase EC1.14.19.9 (Uniprot ID: |
| | | Q8KHZ8) |
| 35 | LaRebF | Lechevalieria aerocolonigenes flavin |
| | (protein) | reductase (Uniprot ID: Q8KI76) |
| 36 | OcINMT | Indolethylamine N-methyltransferase from |
| | (protein) | Oryctolagus cuniculus (Uniprot Seq. ID: |
| | | O97972) |
| 37 | SrPyrH | Codon optimized for S. cerevisiae |
| | (DNA) | |
| 38 | SttH | Codon optimized for S. cerevisiae |
| | (DNA) | |
| 39 | LaRebH | Codon optimized for S. cerevisiae |
| | (DNA) | |
| 40 | LaRebF | Codon optimized for S. cerevisiae |
| | (DNA) | |
| 41 | OcINMT | Codon optimized for S. cerevisiae |
| | (DNA) | |
| 42 | PcCyb5 | Psilocybe cubensis putative Cytochrome b5 |
| | (protein) | |
| 43 | PcCyb5 | (Codon optimized for S. cerevisiae) |
| | (DNA) | |
| 44 | Erg4 | S. cerevisiae Sterol reductase (Uniprot ID: |
| | (protein) | P25340) |
| 45 | Erg4 (DNA) | |
| 46 | Spe2 | S. cerevisiae SAM decarboxylase (Uniprot |
| | (protein) | ID: P21182) |
| 47 | Spe2 (DNA) | |
| 48 | CcCmdE | Chondromyces crocatus |
| | (protein) | Q0VZ69 |
| 49 | CcCmdE | Codon optimized for S. cerevisiae |
| | (DNA) | |
| 50 | PfPrnA | Pseudomonas fluorescens Tryptophan 7- |
| | (protein) | halogenase EC1.14.19.9 (Uniprot ID: |
| | | P95480) |
| 51 | SaThal | Streptomyces albogriseolus Tryptophan 6- |
| | (protein) | halogenase EC 1.14.19.59 (Uniprot ID: |
| | | A1E280) |
| 52 | DdChlA | Dictyostelium discoideum Tryptophan |
| | (protein) | halogenase |
| 53 | KtzQ | Kutzneria sp. 744 Tryptophan 7-halogenase |
| | (protein) | (NCBI accession number: EU074211.1) |
| 54 | KtzR | Kutzneria sp. 744 Tryptophan 6-halogenase |
| | (protein) | (NCBI accession number: ABV56598.1) |
| 55 | PfPrnA | Codon optimized for S. cerevisiae |
| | (DNA) | |
| 56 | SaThal | Codon optimized for S. cerevisiae |
| | (DNA) | |
| 57 | DdChlA | Codon optimized for S. cerevisiae |
| | (DNA) | |
| 58 | KtzQ | Codon optimized for S. cerevisiae |
| | (DNA) | |
| 59 | KtzR | Codon optimized for S. cerevisiae |
| | (DNA) | |

### REFERENCES

Averesch, N. J. H. and Krömer, J. O., 2018. Metabolic Engineering of the Shikimate Pathway for Production of Aromatics and Derived Compounds—Present and Future Strain Construction Strategies. Frontiers in Bioengineering and Biotechnology 6, 32.
Bogenschutz, M. P., Forcehimes, A. A., Pommy, J. A., Wilcox, C. E., Barbosa, P., Strassman, R. J., 2015. Psilocybin-assisted treatment for alcohol dependence: A proof-of-concept study. J. Psychopharmacol. 29, 289-299
Brown, S., Clastre, M., Courdavault, V., O'Connor, S. E., 2015. De novo production of the plant-derived alkaloid strictosidine in yeast. Proc. Natl. Acad. Sci. 112, 3205-3210.
Carhart-Harris1, R. L., & M. Bolstridge1, 2, & C. M. J. Day1, 2, & J. Rucker1, 3, 4, &, Watts1, R., Erritzoe1, & D. E., Kaelen1, & M., Giribaldi1, & B., Bloomfield5, & M., Pilling6, & S., Rickard7, & J. A., & B. Forbes8 & A. Feilding9 & D. Taylor10 & H. V. Curran6, 11 & D. J. Nutt 1, 2018. Psilocybin with psychological support for treatment-resistant depression: six-month follow-up. Psychopharmacology (Berl). 235, 399-408.
Entian, K. D., Kötter, P., 2007. 25 Yeast Genetic Strain and Plasmid Collections. Methods Microbiol. 36, 629-666. https://doi.org/10.1016/50580-9517(06)36025-4Fricke et al., 2017
Germann, S. M., et al., 2016. Glucose-based microbial production of the hormone melatonin in yeast Saccharomyces cerevisiae. Biotechnol. 11, 717-724.
Gietz, R. D., Woods, R. A., 2002. Transformation of yeast by lithium acetate/single-stranded carrier DNA/polyethylene glycol method. Methods Enzymol. 350, 87-96.
Graf, R., Mehmann, B., Braus, G. H., 1993. Analysis of feedback-resistant anthranilate synthases from Saccharomyces cerevisiae. J. Bacteriol. 175, 1061-1068. https://doi.org/10.1128/jb.175.4.1061-1068.1993
Grob, C. S., Danforth, A. L., Chopra, G. S., Hagerty, M., McKay, C. R., Halberstad, A. L., Greer, G. R., 2011. Pilot study of psilocybin treatment for anxiety in patients with advanced-stage cancer. Arch. Gen. Psychiatry 68, 71-78.
Grote, A., Hiller, K., Scheer, M., Münch, R., Nörtemann, B., Hempel, D. C., Jahn, D., 2005. JCat: A novel tool to adapt codon usage of a target gene to its potential expression host. Nucleic Acids Res. 33, 526-531.
Jensen, N., Gartz, J., Laatsch, H., 2006. Aeruginascin, a Trimethylammonium Analogue of Psilocybin from the Hallucinogenic Mushroom Inocybe aeroginascens. Planta. Med. 72: 665-666.
Jessop-Fabre, M. M., Jakoc̆iūnas, T., Stovicek, V., Dai, Z., Jensen, M. K., Keasling, J. D., Borodina, I., 2016. EasyClone-MarkerFree: A vector toolkit for marker-less integration of genes into Saccharomyces cerevisiae via CRISPR-Cas9. Biotechnol. J. 11, 1110-1117.
Li, M., Schneider, K., Kristensen, M., Borodina, I., Nielsen, J., 2016. Engineering yeast for high-level production of stilbenoid antioxidants. Sci. Rep. 6, 1-8.
Luttik, M. A. H., Vuralhan, Z., Suir, E., Braus, G. H., Pronk, J. T., Daran, J. M., 2008. Alleviation of feedback inhibition in Saccharomyces cerevisiae aromatic amino acid biosynthesis: Quantification of metabolic impact. Metab. Eng. 10, 141-153.
Maury, J., Germann, S. M., Baallal Jacobsen, S. A., Jensen, N. B., Kildegaard, K. R., Herrgàrd, M. J., Schneider, K., Koza, A., Forster, J., Nielsen, J., Borodina, I., 2016. EasyCloneMulti: A set of vectors for simultaneous and multiple genomic integrations in Saccharomyces cerevisiae. PLoS One 11, 1-22.
Nichols, D. E., Frescas, S., 1999. Improvements to the Synthesis of Psilocybin and a Facile Method for Preparing the 0-Acetyl Prodrug of Psilocin. Synthesis (Stuttg). 6, 935-938.
Nijkamp, J. F., van den Broek, M., Datema, E., de Kok, S., Bosman, L., Luttik, M. A., Daran-Lapujade, P., Vongsangnak, W., Nielsen, J., Heijne, W. H. M., Klaassen, P., Paddon, C. J., Platt, D., Kötter, P., van Ham, R. C., Reinders, M. J. T., Pronk, J. T., de Ridder, D., Daran, J. M., 2012. De novo sequencing, assembly and analysis of the genome of the laboratory strain Saccharomyces cerevisiae CEN.PK113-7D, a model for modern industrial biotechnology. Microb. Cell Fact. 11.
Paddon, C. J., Westfall, P. J., Pitera, D. J., Benjamin, K., Fisher, K., McPhee, D., Leavell, M. D., Tai, A., Main, A., Eng, D., Polichuk, D. R., Teoh, K. H., Reed, D. W., Treynor, T., Lenihan, J., Jiang, H., Fleck, M., Bajad, S., Dang, G., Dengrove, D., Diola, D., Dorin, G., Ellens, K. W., Fickes, S., Galazzo, J., Gaucher, S. P., Geistlinger, T., Henry, R., Renault, H., Bassard, J. E., Hamberger, B., Werck-Reichhart, D., 2014. Cytochrome P450-mediated metabolic engineering: Current progress and future challenges. Curr. Opin. Plant Biol. 19, 27-34.
Riaz, N., Wolden, S. L., Gelblum, D. Y., Eric, J., 2016. Long-term follow-up of psilocybinfacilitated smoking cessation 118, 6072-6078.
Rush, A. J., Trivedi, M. H., Wisniewski, S. R., Nierenberg, A. A., Stewart, J. W., Warden, D., Niederehe, G., Thase, M. E., Lavori, P. W., Lebowitz, B. D., McGrath, P. J., Rosenbaum, J. F., Sackeim, H. A., Kupfer, D. J., Luther, J., Fava, M., 2006. Acute and Longer-Term Outcomes in Depressed Outpatients Requiring One. Am. J. Psychiatry 163, 1905-1917.
Suástegui, M., Yu Ng, C., Chowdhury, A., Sun, W., Cao, M., House, E., Maranas, C. D., Shao, Z., 2017. Multilevel engineering of the upstream module of aromatic amino acid biosynthesis in Saccharomyces cerevisiae for high production of polymer and drug precursors. Metab. Eng. 42, 134-144.
Tice, P., 2017. Substance Abuse & Mental Health Servs. Admin., Results from the 2016 National Survey on Drug Use and Health.
Tyls̆, F., Páleníc̆ek, T., Horác̆ek, J., 2014. Psilocybin—Summary of knowledge and new perspectives. Eur. Neuropsychopharmacol. 24, 342-356.
Xu, Z., Yang, Z., Liu, Y., Lu, Y., Chen, K. and Zhu, W., 2014. Halogen bond: Its role beyond drug-target binding affinity for drug discovery and development. Journal of Chemical Information and Modeling, 54(1):69-78.

Item List 1
1. A yeast cell capable of producing 4-hydroxytryptamine and optionally derivatives thereof, said cell expressing:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
   a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
   a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,
   wherein the tryptamine 4-monooxygenase and the cytochrome P450 reductase together catalyse the conversion of tryptamine to 4-hydroxytryptamine,
   whereby the yeast cell is capable of converting tryptophan to 4-hydroxytryptamine.
2. The yeast cell according to item 1, wherein the yeast cell is a Saccharomyces cerevisiae cell.
3. The yeast cell according to any one of the preceding items, wherein the yeast cell is capable of producing 4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
4. The yeast cell according to any one of the preceding items, further expressing a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin,
   whereby the yeast cell is capable of converting 4-hydroxytryptamine to norbaeocystin.
5. The yeast cell according to item 4, wherein the yeast cell is capable of producing norbaeocystin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
6. The yeast cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the yeast cell is capable of converting norbaeocystin to baeocystin, wherein optionally the baeocystin is converted spontaneously to norpsilocin.
7. The yeast cell according to item 6, wherein the yeast cell is capable of producing baeocystin and optionally norpsilocin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
8. The yeast cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and baeocystin to psilocybin,
   whereby the yeast cell is capable of converting norbaeocystin to psilocybin,
   wherein optionally the psilocybin is converted spontaneously to psilocin.
9. The yeast cell according to item 8, wherein the yeast cell is capable of producing psilocybin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
10. The yeast cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, baeocystin to psilocybin, and psilocybin to aeruginascin,
   whereby the yeast cell is capable of converting norbaeocystin to aeruginascin,
   wherein optionally the aeruginascin is converted spontaneously to dephosphorylated aeruginascin.
11. The yeast cell according to any one of the preceding items, wherein the yeast cell is capable of producing aeruginascin and optionally dephosphorylated aeruginascin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
12. The yeast cell according to any one of the preceding items, further expressing a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
13. The yeast cell according to any one of the preceding items, wherein the yeast cell is capable of producing N-acetyl-4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
14. The yeast cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is under the control of an inducible promoter.
15. The yeast cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is codon-optimised for the yeast cell.
16. The yeast cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is present in high copy number.
17. The yeast cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is integrated in the genome of the yeast cell.
18. The yeast cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is expressed from a vector such as a plasmid.
19. The yeast cell according to any one of the preceding items, further comprising one or more mutations resulting in increased availability of L-tryptophan.
20. The yeast cell according to item 19, wherein the one or more mutations is in one or more genes encoding transcriptional repressor(s) of genes of the aromatic amino acid precursor pathway such as ARO1, ARO2, ARO3 or ARO4.
21. The yeast cell according to item 20, wherein the one or more mutations is a mutation resulting in partial or total loss of activity of the one or more transcriptional repressor(s).
22. The yeast cell according to any one of items 19 to 21, wherein the one or more mutations is one or more of:
   a mutation in ARO4 (SEQ ID NO: 12), such as a K229L mutation;
   a mutation in ARO1 (SEQ ID NO: 13) and/or ARO2 (SEQ ID NO: 14);
   a mutation in CDC19 (SEQ ID NO: 26);
   a mutation in ARO8 (SEQ ID NO: 23) and/or ARO9 (SEQ ID NO: 24);
   a mutation in GLT1 (SEQ ID NO: 25);
   a mutation in TRP2 (SEQ ID NO: 16);
wherein the mutation is a mutation leading to a loss of function of the corresponding protein, such as a deletion, preferably wherein the yeast cell is a Saccharomyces cerevisiae cell.
23. The yeast cell according to any one of items 19 to 22, wherein the yeast cell overexpresses one or more of:
   SER1 (SEQ ID NO: 20);
   SER2 (SEQ ID NO: 21);
   SER3 (SEQ ID NO: 22);
   SER33 (SEQ ID NO: 23);
   STB5 (SEQ ID NO: 26);
   POS5 (SEQ ID NO: 27);
   ZWF1 (SEQ ID NO: 29),
preferably wherein the yeast cell is a Saccharomyces cerevisiae cell.
24. The yeast cell according to any one of items 20 to 23, wherein the transcriptional repressor is Ric1.
25. The yeast cell according to any one of the preceding items, further expressing a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto.
26. The yeast cell according to any one of the preceding items, further comprising one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).
27. A method of producing 4-hydroxytryptamine and optionally derivatives thereof in a yeast cell, said method comprising the steps of providing a yeast cell and incubating said yeast cell in a medium, wherein the yeast cell expresses:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
   a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
   a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.
28. The method according to item 27, wherein the method is for producing norbaeocystin and the yeast cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.
29. The method according to any one of items 27 to 28, wherein the method is for producing baeocystin and optionally norpsilocin and the yeast cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto
30. The method according to any one of items 27 to 29, wherein the method is for producing N-acetyl-4-hydroxytryptamine and the yeast cell further expresses a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
31. The method according to any one of items 27 to 30, wherein the method is for producing psilocybin and optionally psilocin and the yeast cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
32. The method according to any one of items 27 to 31, wherein the method is for producing aeruginascin and optionally dephosphorylated aeruginascin and the yeast cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
33. The method according to any one of items 27 to 32, wherein the medium comprises tryptophan and/or wherein the yeast cell is capable of synthesising tryptophan.
34. The method according to any one of items 27 to 33, wherein the yeast cell is as defined in any one of items 1 to 26.
35. The method according to any one of items 27 to 34, wherein the medium is supplemented with at least 1 g/L glutamine, such as at least 2 g/L, such as at least 3 g/L glutamine, such as at least 4 g/L glutamine, such as at least 5 g/L glutamine, such as at least 6 g/L glutamine, such as at least 7 g/L glutamine, such as at least 8 g/L glutamine, such as at least 9 g/L glutamine, such as at least 10 g/L glutamine, or more.
36. The method according to any one of items 27 to 35, wherein 4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
37. The method according to any one of items 27 to 36, wherein norbaeocystin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
38. The method according to any one of items 27 to 37, wherein baeocystin and optionally norpsilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
39. The method according to any one of items 27 to 38, wherein N-acetyl-4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
40. The method according to any one of items 27 to 39, wherein psilocybin and optionally psilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
41. The method according to any one of items 27 to 40, wherein aeruginascin and optionally dephosphorylated aeruginascin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
42. The method according to any one of items 27 to 41, wherein the cell further expresses a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto.
43. The method according to any one of items 27 to 42, wherein the cell further comprises one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).
44. The method according to any one of items 27 to 43, further comprising a step of recovering the 4-hydroxytryptamine.
45. The method according to any one of items 27 to 44, further comprising a step of recovering the norbaeocystin.
46. The method according to any one of items 27 to 45, further comprising a step of recovering the baeocystin and optionally the norpsilocin.
47. The method according to any one of items 27 to 46, further comprising a step of recovering the N-acetyl-4-hydroxytryptamine.
48. The method according to any one of items 27 to 47, further comprising a step of recovering the psilocybin and optionally the psilocin.
49. The method according to any one of items 27 to 48, wherein the method further comprises a step of recovering the aeruginascin and optionally the dephosphorylated aeruginascin.
50. 4-hydroxytryptamine, norbaeocystin, baeocystin, norpsilocin, psilocybin, psilocin, aeruginascin, dephosphorylated aeruginascin or N-acetyl-4-hydroxytryptamine obtainable by a method according to any one of items 27 to 49.
51. A nucleic acid construct for modifying a yeast cell, said construct comprising:
   a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto;
   a second polynucleotide encoding a tryptamine 4-monooxygenase (EC 1.14.99.59) (SEQ ID NO: 7), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto; and
   a third polynucleotide encoding a cytochrome P450 reductase (EC 1.6.2.4) (SEQ ID NO:8), preferably a heterologous cytochrome P450 reductase such as PcCpr (SEQ ID NO: 3) or a functional variant thereof having at least 80% homology, such as at least 85%, such as at least 90%, such as at least 95% homology thereto.
52. The nucleic acid construct according to item 51, further comprising a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.
53. The nucleic acid construct according to any one of items 51 to 52, further comprising a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
54. The nucleic acid construct according to any one of items 51 to 53, further comprising a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
55. The nucleic acid construct according to any one of items 51 to 54, wherein the first polynucleotide comprises or consists of SEQ ID NO: 6 or a variant thereof having at least 80% homology, such as at least 85%, such as at least 90%, such as at least 95% homology thereto.
56. The nucleic acid construct according to any one of items 51 to 55, wherein the second polynucleotide comprises or consists of SEQ ID NO: 7 or a homologue thereof having at least 80% homology, such as at least 85%, such as at least 90%, such as at least 95% homology thereto.
57. The nucleic acid construct according to any one of items 51 to 56, wherein the third polynucleotide comprises or consists of SEQ ID NO: 8 or a homologue thereof having at least 80% homology, such as at least 85%, such as at least 90%, such as at least 95% homology thereto.
58. The nucleic acid construct according to any one of items 51 to 57, wherein the fourth polynucleotide comprises or consists of SEQ ID NO: 9 or a homologue thereof having at least 80% homology, such as at least 85%, such as at least 90%, such as at least 95% homology thereto.
59. The nucleic acid construct according to any one of items 51 to 58, wherein the fifth polynucleotide comprises or consists of SEQ ID NO: 10 or a homologue thereof having at least 80% homology, such as at least 85%, such as at least 90%, such as at least 95% homology thereto.
60. The nucleic acid construct according to any one of items 51 to 59, wherein the sixth polynucleotide comprises or consists of SEQ ID NO: 30 or a homologue thereof having at least 80% homology, such as at least 85%, such as at least 90%, such as at least 95% homology thereto.
61. The nucleic acid construct according to any one of items 51 to 60, wherein one or more of the first, second, third, fourth, fifth and sixth polynucleotide(s) is/are codon-optimised for said yeast cell.
62. The nucleic acid construct according to any one of items 51 to 61, comprising or consisting of one or more vectors.
63. The yeast cell according to any one of items 1 to 26, wherein the yeast cell comprises a nucleic acid construct according to any one of item 51 to 62.
64. A kit of parts comprising:
   the yeast cell according to any one of items 1 to 26 and instructions for use; and/or
   the nucleic acid construct according to any one of items 51 to 62 and instructions for use; and optionally the yeast cell to be modified.
65. A cell capable of producing N-methyltryptamine, N,N-dimethyltryptamine and/or N,N,N-trimethyltryptamine, preferably wherein the cell is as defined in any one of the preceding items, said cell expressing:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine; and
   an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, wherein the indole N-methyltransferase is capable of converting tryptamine to N-methyltryptamine, to N,N-dimethyltryptamine, and/or to N,N,N-trimethyltryptamine,
   whereby the cell is capable of producing N-methyltryptamine, N,N-dimethyltryptamine, and/or N,N,N-trimethyltryptamine.
66. A method for producing N-methyltryptamine, N,N-dimethyltryptamine and/or N,N,N-trimethyltryptamine in a cell, preferably wherein the cell is as defined in any one of the preceding items, said method comprising the steps of providing a cell and incubating said cell in a medium, wherein the cell expresses:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine; and
   an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, wherein the indole N-methyltransferase is capable of converting tryptamine to N-methyltryptamine, to N,N-dimethyltryptamine, and/or to N,N,N-trimethyltryptamine.
67. The method according to item 66, wherein N-methyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L.
68. The method according to any one of items 66 to 67, wherein N,N-dimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L.
69. The method according to any one of items 66 to 68, wherein N,N,N-trimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
70. A nucleic acid construct for modifying a cell, wherein the cell is as defined in any one of the preceding items, said construct comprising:
   a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto, such as a first polynucleotide comprising or consisting of SEQ ID NO: 6 or a homologue thereof having at least 80% homology, such as at least 85%, such as at least 90%, such as at least 95% homology thereto
   a seventh polynucleotide encoding an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, such as a seventh polynucleotide comprising or consisting of SEQ ID NO: 36 or a homologue thereof having at least 80% homology, such as at least 85%, such as at least 90%, such as at least 95% homology thereto.
71. The nucleic acid construct according to item 70, wherein the first and/or the seventh polynucleotide(s) is/are codon-optimized for said cell.
72. The nucleic acid construct according to any one of items 70 to 71, comprising or consisting of one or more vectors.
73. A kit of parts comprising:
   the cell according to item 65 and instructions for use, preferably wherein the cell is a microorganism or a plant cell; and/or
   the nucleic acid construct according to any one of items 70 to 72 and instructions for use; and optionally the cell to be modified.

Item list 2
1. A cell capable of producing 4-hydroxytryptamine and optionally derivatives thereof, said cell expressing:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
   a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
   a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,
   wherein the tryptamine 4-monooxygenase and the cytochrome P450 reductase together catalyse the conversion of tryptamine to 4-hydroxytryptamine,
   whereby the cell is capable of converting tryptophan to 4-hydroxytryptamine.
2. The cell according to item 1, wherein the cell is a microorganism or a plant cell.
3. The cell according to item 2, wherein the microorganism is a fungus or a bacteria.
4. The cell according to item 3, wherein the fungus is selected from the group consisting of a fungus belonging to the genus of Aspergillus, e.g. A. niger, A. awamori, A. oryzae, A. nidulans, a yeast belonging to the genus of Saccharomyces, e.g. S. cerevisiae, S. kluyveri, S. bayanus, S. exiguus, S. sevazzi, S. uvarum, S. boulardii, a yeast belonging to the genus Kluyveromyces, e.g. K. lactis, K. marxianus var. marxianus, K. thermotolerans, a yeast belonging to the genus Candida, e.g. C. utilis C. tropicalis, C. albicans, C. lipolytica, C. versatilis, a yeast belonging to the genus Pichia, e.g. P. stipidis, P. pastoris, P. sorbitophila, other yeast genera such as Cryptococcus (e.g. C. aerius), Debaromyces (e.g. D. hansenii), Hansenula, Pichia (e.g. P. pastoris), Yarrowia (e.g. Y. lipolytica), Zygosaccharomyces (e.g. Z. bailii), Torulaspora (e.g. T. delbrueckii), Schizosaccharomyces (e.g. S. pombe), Brettanomyces (e.g. B. bruxellensis), Penicillium, Rhizopus, Fusarium, Fusidium, Gibberella, Mucor, Mortierella, and Trichoderma.
5. The cell according to item 3, wherein the bacteria is selected from the group consisting of a species belonging to the genus Bacillus (e.g. B. subtilis), a species belonging to the genus Escherichia (e.g. E. coli), a species belonging to the genus Lactobacillus (e.g. L. casei), a species belonging to the genus Lactococcus (e.g. L. lactis), a species belonging to the genus Corynebacterium (e.g. C. glutamicum), a species belonging to the genus Acetobacter, a species belonging to the genus Acinetobacter, a species belonging to the genus Pseudomonas (e.g. P. putida), and a species belonging to the genus Streptomyces (e.g. S. coelicolor).
6. The cell according to item 2, wherein the plant cell is selected from the group consisting of a species belonging to the genus Arabidopsis (e.g. A. thaliana), a species belonging to the genus Zea (e.g. Z. mays), a species belonging to the genus Medicago (e.g. M. truncatula), a species belonging to the genus Nicotiana (e.g. N. tabacum) and a species belonging to the genus Glycine (e.g. G. Max).
7. The cell according to any one of the preceding items, wherein the cell is capable of producing 4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
8. The cell according to any one of the preceding items, further expressing a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin,
   whereby the cell is capable of converting 4-hydroxytryptamine to norbaeocystin.
9. The cell according to item 8, wherein the cell is capable of producing norbaeocystin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
10. The cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the cell is capable of converting norbaeocystin to baeocystin, wherein optionally the baeocystin is converted spontaneously to norpsilocin.
11. The cell according to item 10, wherein the cell is capable of producing baeocystin and optionally norpsilocin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
12. The cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and baeocystin to psilocybin,
   whereby the cell is capable of converting norbaeocystin to psilocybin, wherein optionally the psilocybin is converted spontaneously to psilocin.
13. The cell according to item 12, wherein the cell is capable of producing psilocybin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
14. The cell according to any one of the preceding items, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, baeocystin to psilocybin, and psilocybin to aeruginascin,
   whereby the cell is capable of converting norbaeocystin to aeruginascin,
   wherein optionally the aeruginascin is converted spontaneously to dephosphorylated aeruginascin.
15. The cell according to any one of the preceding items, wherein the cell is capable of producing aeruginascin and optionally dephosphorylated aeruginascin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
16. The cell according to any one of the preceding items, further expressing a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
17. The cell according to any one of the preceding items, wherein the cell is capable of producing N-acetyl-4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
18. The cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is under the control of an inducible promoter.
19. The cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is codon-optimised for the cell.
20. The cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is present in high copy number.
21. The cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is integrated in the genome of the cell.
22. The cell according to any one of the preceding items, wherein one or more of the genes encoding the tryptophan decarboxylase, the tryptamine 4-monooxygenase, the cytochrome P450 reductase, the 4-hydroxytryptamine kinase, the serotonin N-acetyltransferase and/or the psilocybin synthase is expressed from a vector such as a plasmid.
23. The cell according to any one of the preceding items, further comprising one or more mutations resulting in increased availability of L-tryptophan.
24. The cell according to item 23, wherein the one or more mutations is in one or more genes encoding transcriptional repressor(s) of genes of the aromatic amino acid precursor pathway such as ARO1, ARO2, ARO3 or ARO4.
25. The cell according to item 24, wherein the one or more mutations is a mutation resulting in partial or total loss of activity of the one or more transcriptional repressor(s).
26. The cell according to any one of items 23 to 25, wherein the one or more mutations is one or more of:
   a mutation in ARO4 (SEQ ID NO: 12), such as a K229L mutation;
   a mutation in ARO1 (SEQ ID NO: 13) and/or ARO2 (SEQ ID NO: 14);
   a mutation in CDC19 (SEQ ID NO: 26);
   a mutation in ARO8 (SEQ ID NO: 23) and/or ARO9 (SEQ ID NO: 24);
   a mutation in GLT1 (SEQ ID NO: 25);
   a mutation in TRP2 (SEQ ID NO: 16);
wherein the mutation is a mutation leading to a loss of function of the corresponding protein, such as a deletion, such as wherein the cell is a Saccharomyces cerevisiae cell.
27. The cell according to any one of items 23 to 26, wherein the cell overexpresses one or more of:
   SER1 (SEQ ID NO: 20);
   SER2 (SEQ ID NO: 21);
   SER3 (SEQ ID NO: 22);
   SER33 (SEQ ID NO: 23);
   STB5 (SEQ ID NO: 26);
   POS5 (SEQ ID NO: 27);
   ZWF1 (SEQ ID NO: 29),
such as wherein the cell is a Saccharomyces cerevisiae cell.
28. The cell according to any one of items 23 to 27, wherein the transcriptional repressor is Ric1.
29. The cell according to any one of the preceding items, further expressing a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto.
30. The cell according to any one of the preceding items, further comprising one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).
31. A method of producing 4-hydroxytryptamine and optionally derivatives thereof in a cell, said method comprising the steps of providing a cell and incubating said cell in a medium, wherein the cell expresses:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
   a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
   a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.
32. The method according to item 31, wherein the method is for producing norbaeocystin and the cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.
33. The method according to any one of items 31 to 32, wherein the method is for producing baeocystin and optionally norpsilocin and the cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
34. The method according to any one of items 31 to 33, wherein the method is for producing N-acetyl-4-hydroxytryptamine and the cell further expresses a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
35. The method according to any one of items 31 to 34, wherein the method is for producing psilocybin and optionally psilocin and the cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
36. The method according to any one of items 31 to 35, wherein the method is for producing aeruginascin and optionally dephosphorylated aeruginascin and the cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
37. The method according to any one of items 31 to 36, wherein the medium comprises tryptophan and/or wherein the cell is capable of synthesising tryptophan.
38. The method according to any one of items 31 to 37, wherein the cell is as defined in any one of items 1 to 28.
39. The method according to any one of items 31 to 38, wherein the medium is supplemented with at least 1 g/L glutamine, such as at least 2 g/L, such as at least 3 g/L glutamine, such as at least 4 g/L glutamine, such as at least 5 g/L glutamine, such as at least 6 g/L glutamine, such as at least 7 g/L glutamine, such as at least 8 g/L glutamine, such as at least 9 g/L glutamine, such as at least 10 g/L glutamine, or more.
40. The method according to any one of items 31 to 39, wherein 4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
41. The method according to any one of items 31 to 40, wherein norbaeocystin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
42. The method according to any one of items 31 to 41, wherein baeocystin and optionally norpsilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
43. The method according to any one of items 31 to 42, wherein N-acetyl-4-hydroxytryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
44. The method according to any one of items 31 to 43, wherein psilocybin and optionally psilocin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
45. The method according to any one of items 31 to 44, wherein aeruginascin and optionally dephosphorylated aeruginascin is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
46. The method according to any one of items 31 to 45, wherein the yeast cell further expresses a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto.
47. The method according to any one of items 31 to 46, wherein the yeast cell further comprises one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).
48. The method according to any one of items 31 to 47, further comprising a step of recovering the 4-hydroxytryptamine.
49. The method according to any one of items 31 to 48, further comprising a step of recovering the norbaeocystin.
50. The method according to any one of items 31 to 49, further comprising a step of recovering the baeocystin and optionally the norpsilocin.
51. The method according to any one of items 31 to 50, further comprising a step of recovering the N-acetyl-4-hydroxytryptamine.
52. The method according to any one of items 31 to 51, further comprising a step of recovering the psilocybin and optionally the psilocin.
53. The method according to any one of items 31 to 52, wherein the method further comprises a step of recovering the aeruginascin and optionally the dephosphorylated aeruginascin.
54. 4-hydroxytryptamine, norbaeocystin, baeocystin, norpsilocin, psilocybin, psilocin, aeruginascin, dephosphorylated aeruginascin or N-acetyl-4-hydroxytryptamine obtainable by a method according to any one of items 31 to 53.
55. A nucleic acid construct for modifying a cell, said construct comprising:
   a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto;
   a second polynucleotide encoding a tryptamine 4-monooxygenase (EC 1.14.99.59) (SEQ ID NO: 7), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto; and
   a third polynucleotide encoding a cytochrome P450 reductase (EC 1.6.2.4) (SEQ ID NO:8), preferably a heterologous cytochrome P450 reductase such as PcCpr (SEQ ID NO: 3) or a functional variant thereof having at least 80% homology, such as at least 85%, such as at least 90%, such as at least 95% homology thereto.
56. The nucleic acid construct according to item 55, further comprising a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.
57. The nucleic acid construct according to any one of items 55 to 56, further comprising a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.
58. The nucleic acid construct according to any one of items 55 to 57, further comprising a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.
59. The nucleic acid construct according to any one of items 55 to 58, wherein the first polynucleotide comprises or consists of SEQ ID NO: 6 or a variant thereof having at least 80% homology, such as at least 85%, such as at least 90%, such as at least 95% homology thereto.
60. The nucleic acid construct according to any one of items 55 to 59, wherein the second polynucleotide comprises or consists of SEQ ID NO: 7 or a homologue thereof having at least 80% homology, such as at least 85%, such as at least 90%, such as at least 95% homology thereto.
61. The nucleic acid construct according to any one of items 55 to 60, wherein the third polynucleotide comprises or consists of SEQ ID NO: 8 or a homologue thereof having at least 80% homology, such as at least 85%, such as at least 90%, such as at least 95% homology thereto.
62. The nucleic acid construct according to any one of items 55 to 61, wherein the fourth polynucleotide comprises or consists of SEQ ID NO: 9 or a homologue thereof having at least 80% homology, such as at least 85%, such as at least 90%, such as at least 95% homology thereto.
63. The nucleic acid construct according to any one of items 55 to 62, wherein the fifth polynucleotide comprises or consists of SEQ ID NO: 10 or a homologue thereof having at least 80% homology, such as at least 85%, such as at least 90%, such as at least 95% homology thereto.
64. The nucleic acid construct according to any one of items 55 to 63, wherein the sixth polynucleotide comprises or consists of SEQ ID NO: 30 or a homologue thereof having at least 80% homology, such as at least 85%, such as at least 90%, such as at least 95% homology thereto.
65. The nucleic acid construct according to any one of items 55 to 64, wherein one or more of the first, second, third, fourth, fifth and sixth polynucleotide(s) is/are codon-optimised for said cell.
66. The nucleic acid construct according to any one of items 55 to 65, comprising or consisting of one or more vectors.
67. The cell according to any one of items 1 to 30, wherein the cell comprises a nucleic acid construct according to any one of items 55 to 66.
68. A kit of parts comprising:
   the cell according to any one of items 1 to 30 and instructions for use; and/or
   the nucleic acid construct according to any one of items 55 to 66 and instructions for use; and optionally the cell to be modified.
69. A method for producing N-methyltryptamine, N,N-dimethyltryptamine and/or N,N,N-trimethyltryptamine in a yeast cell, preferably wherein the yeast cell is as defined in any one of the preceding items, said method comprising the steps of providing a yeast cell and incubating said cell in a medium, wherein the yeast cell expresses:
   a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine; and
   an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, wherein the indole N-methyltransferase is capable of converting tryptamine to N-methyltryptamine, to N,N-dimethyltryptamine, and/or to N,N,N-trimethyltryptamine.
70. The method according to item 69, wherein N-methyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L.
71. The method according to any one of items 69 to 70, wherein N,N-dimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L.
72. The method according to any one of items 69 to 71, wherein N,N,N-trimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.
73. A nucleic acid construct for modifying a yeast cell, wherein the yeast cell is as defined in any one of the preceding items, said construct comprising:
   a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto, such as a first polynucleotide comprising or consisting of SEQ ID NO: 6 or a homologue thereof having at least 80% homology, such as at least 85%, such as at least 90%, such as at least 95% homology thereto
   a seventh polynucleotide encoding an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, such as a seventh polynucleotide comprising or consisting of SEQ ID NO: 36 or a homologue thereof having at least 80% homology, such as at least 85%, such as at least 90%, such as at least 95% homology thereto.
74. The nucleic acid construct according to item 73, wherein the first and/or the seventh polynucleotide(s) is/are codon-optimized for said cell.
75. The nucleic acid construct according to any one of items 73 to 74, comprising or consisting of one or more vectors.
76. A kit of parts comprising:
   the cell according to item 65 and instructions for use, preferably wherein the cell is a microorganism or a plant cell; and/or
   the nucleic acid construct according to any one of items 73 to 75 and instructions for use; and optionally the cell to be modified.

1. A cell capable of producing a halogenated tryptophan, wherein the halogenated tryptophan is a tryptophan substituted with one, two or three halogen atoms, and optionally derivatives thereof, in the presence of a halogen or derivatives thereof, said cell expressing at least one of:
a tryptophan-2-halogenase (EC 1.14.14), preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto,
and optionally expressing a flavin reductase (EC 1.5.1.30), preferably a heterologous flavin reductase, such as LaRebF (SEQ ID NO: 35) or a functional variant thereof having at least 80% homology, such as at least 85%, such as at least 90%, such as at least 95% homology thereto,
whereby the cell is capable of converting tryptophan into a halogenated tryptophan and optionally derivatives thereof, a dihalogenated tryptophan and optionally derivatives thereof, or a trihalogenated tryptophan and optionally derivatives thereof,
preferably wherein the cell is a microorganism or a plant cell.

2. The cell according to claim 1, wherein the tryptophan is 2-halogenated, 5-halogenated, 6-halogenated or 7-halogenated by a halogen selected from the group consisting of fluorine, bromine, iodine and chlorine.

3. The cell according to any one of the preceding claims, wherein the derivative of tryptophan is 2,5-dihalogenated, 2,6-dihalogenated, 2,7-dihalogenated, 5,6-dihalogenated, 5,7-dihalogenated or 6,7-dihalogenated by two halogens independently selected from the group consisting of fluorine, bromine, iodine and chlorine.

4. The cell according to any one of the preceding claims, wherein the derivative of tryptophan is 2,5,6-trihalogenated, 2,5,7-trihalogenated, 2,6,7-trihalogenated or 5,6,7-trihalogenated by three halogens independently selected from the group consisting of fluorine, bromine, iodine and chlorine.

5. The cell according to any one of the preceding claims, wherein the cell is a eukaryotic cell or a bacteria or the plant cell is a microalgae cell.

6. The cell according to claim 5, wherein the eukaryotic cell is selected from the group consisting of a eukaryote belonging to the genus of Aspergillus, such as A. niger, A. awamori, A. oryzae, and A. nidulans.

7. The cell according to claim 5, wherein the bacteria is selected from the group consisting of a species belonging to the genus Bacillus, such as B. subtilis, a species belonging to the genus Escherichia, such as E. coli, a species belonging to the genus Lactobacillus, such as L. casei, a species belonging to the genus Lactococcus, such as L. lactis, a species belonging to the genus Corynebacterium, such as C. glutamicum, a species belonging to the genus Acetobacter, a species belonging to the genus Acinetobacter, a species belonging to the genus Pseudomonas, such as P. putida, and a species belonging to the genus Streptomyces, such as S. coelicolor.

8. The cell according to claim 5, wherein the plant is selected from the group consisting of a species belonging to the genus Arabidopsis, such as A. thaliana, a species belonging to the genus Zea, such as Z. mays, a species belonging to the genus Medicago, such as M. truncatula, a species belonging to the genus Nicotiana, such as N. tabacum, and a species belonging to the genus Glycine, such as G. max.

9. The cell according to claim 5, wherein the cell is a yeast cell.

10. The cell according to any one of the preceding claims, wherein the cell is a yeast cell belonging to the genus of Saccharomyces, such as S. cerevisiae, S. kluyveri, S. bayanus, S. exiguus, S. sevazzi, S. uvarum, S. boulardii, a yeast belonging to the genus Kluyveromyces, such as K. lactis, K. marxianus var. marxianus, K. thermotolerans, a yeast belonging to the genus Candida, such as C. utilis, C. tropicalis, C. albicans, C. lipolytica, C. versatilis, a yeast belonging to the genus Pichia, such as P. stipidis, P. pastoris, P. sorbitophila, other yeast genera such as Cryptococcus, such as C. aerius, Debaromyces, such as D. hansenii, Hansenula, Pichia, such as P. pastoris, Yarrowia, such as Y. lipolytica, Zygosaccharomyces, such as Z. bailii, Torulaspora, such as T. delbrueckii, Schizosaccharomyces, such as S. pombe, Brettanomyces, such as B. bruxellensis, Penicillium, Rhizopus, Fusarium, Fusidium, Gibberella, Mucor, Mortierella, and Trichoderma.

11. The cell according to any one of the preceding claims, wherein the cell is capable of producing a 2-halogenated, a 5-halogenated, a 6-halogenated, or a 7-halogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

12. The cell according to any one of the preceding claims, wherein the cell is capable of producing a 2,5-dihalogenated, a 2,6-dihalogenated, a 2,7-dihalogenated, a 5,6-dihalogenated, a 5,7-dihalogenated or a 6,7-dihalogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

13. The cell according to any one of the preceding claims, wherein the cell is capable of producing a 2,5,6-trihalogenated, a 2,5,7-trihalogenated, a 2,6,7-trihalogenated or a 5,6,7-trihalogenated tryptophan with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

14. The cell according to any one of the preceding claims, further expressing a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting the halogenated tryptophan to a corresponding halogenated tryptamine, whereby the cell is capable of producing the halogenated tryptamine and optionally derivatives thereof, wherein the halogenated tryptamine or derivative thereof is a tryptamine or a tryptamine derivative substituted with one, two or three halogen atoms.

15. The cell according to any one of the preceding claims, wherein the cell is capable of producing a 2-halogenated tryptamine, a 5-halogenated tryptamine, a 6-halogenated tryptamine, and/or a 7-halogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

16. The cell according to any one of the preceding claims, wherein the cell is capable of producing a 2,5-dihalogenated tryptamine, a 2,6-dihalogenated tryptamine, a 2,7-dihalogenated tryptamine, a 5,6-dihalogenated tryptamine, a 5,7-dihalogenated tryptamine, and/or a 6,7-dihalogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

17. The cell according to any one of the preceding claims, wherein the cell is capable of producing a 2,5,6-trihalogenated tryptamine, a 2,5,7-trihalogenated tryptamine, a 2,6,7-trihalogenated tryptamine and/or a 5,6,7-trihalogenated tryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

18. The cell according to any one of the preceding claims, additionally expressing an indole N-methyltransferase (EC 2.1.1.49), preferably a heterologous indole N-methyltransferase, such as OcINMT (SEQ ID NO: 36) or a functional variant thereof having at least 80% homology, such as at least 85%, such as at least 90%, such as at least 95% homology thereto, wherein the indole N-methyltransferase is capable of converting the halogenated tryptamine to a corresponding halogenated N-methyltryptamine, a corresponding halogenated N,N-dimethyltryptamine, and/or a corresponding halogenated N,N,N-trimethyltryptamine, whereby the cell is capable of producing the halogenated N-methyltryptamine, the halogenated N,N-dimethyltryptamine, the halogenated N,N,N-trimethyltryptamine, and optionally derivatives thereof, wherein the halogenated N-methyltryptamine, the halogenated N,N-dimethyltryptamine, the halogenated N,N,N-trimethyltryptamine, or derivatives thereof, are N-methyltryptamine, N,N-dimethyltryptamine, N,N,N-trimethyltryptamine, or derivatives thereof, substituted with one, two or three halogen atoms.

19. The cell according to any one of the preceding claims, wherein the cell is capable of producing a 2-halogenated N-methyltryptamine, a 2-halogenated N,N-dimethyltryptamine and/or a 2-halogenated N,N,N-trimethyltryptamine, a 5-halogenated N-methyltryptamine, a 5-halogenated N,N-dimethyltryptamine and/or a 5-halogenated N,N,N-trimethyltryptamine, a 6-halogenated N-methyltryptamine, a 6-halogenated N,N-dimethyltryptamine and/or a 6-halogenated N,N,N-trimethyltryptamine, and/or a 7-halogenated N-methyltryptamine, a 7-halogenated N,N-dimethyltryptamine and/or a 7-halogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

20. The cell according to any one of the preceding claims, wherein the cell is capable of producing a 2,5-dihalogenated N-methyltryptamine, a 2,5-dihalogenated N,N-dimethyltryptamine and/or a 2,5-dihalogenated N,N,N-trimethyltryptamine, a 2,6-dihalogenated N-methyltryptamine, a 2,6-dihalogenated N,N-dimethyltryptamine and/or a 2,6-dihalogenated N,N,N-trimethyltryptamine, a 2,7-dihalogenated N-methyltryptamine, a 2,7-dihalogenated N,N-dimethyltryptamine and/or a 2,7-dihalogenated N,N,N-trimethyltryptamine, a 5,6-dihalogenated N-methyltryptamine, a 5,6-dihalogenated N,N-dimethyltryptamine and/or a 5,6-dihalogenated N,N,N-trimethyltryptamine, a 5,7-dihalogenated N-methyltryptamine, a 5,7-dihalogenated N,N-a dimethyltryptamine and/or a 5,7-dihalogenated N,N,N-trimethyltryptamine, and/or a 6,7-dihalogenated N-methyltryptamine, a 6,7-dihalogenated N,N-dimethyltryptamine and/or a 6,7-dihalogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

21. The cell according to any one of the preceding claims, wherein the cell is capable of producing a 2,5,6-trihalogenated N-methyltryptamine, a 2,5,6-trihalogenated N,N-dimethyltryptamine and/or a 2,5,6-trihalogenated N,N,N-trimethyltryptamine, a 2,5,7-trihalogenated N-methyltryptamine, a 2,5,7-trihalogenated N,N-dimethyltryptamine and/or a 2,5,7-trihalogenated N,N,N-trimethyltryptamine, a 2,6,7-trihalogenated N-methyltryptamine, a 2,6,7-trihalogenated N,N-dimethyltryptamine and/or a 2,6,7-trihalogenated N,N,N-trimethyltryptamine, a 5,6,7-trihalogenated N-methyltryptamine, a 5,6,7-trihalogenated N,N-dimethyltryptamine and/or a 5,6,7-trihalogenated N,N,N-trimethyltryptamine, preferably with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

22. The cell according to any one of the preceding claims, wherein the total titer of all produced halogenated compounds is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more, wherein the total titer is the sum of the titers of the 2-halogenated, 5-halogenated, 6-halogenated, 7-halogenated, 2,5-dihalogenated, 2,6-dihalogenated, 2,7-dihalogenated, 5,6-dihalogenated, 5,7-dihalogenated, 6,7-dihalogenated, 2,5,6-trihalogenated, 2,5,7-trihalogenated, 2,6,7-trihalogenated and 5,6,7-trihalogenated tryptophans, tryptamines, N-methyltryptamines, N,N-dimethyltryptamines and N, N, N-trimethyltryptamines.

23. The cell according to any one of the preceding claims, wherein one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is under the control of an inducible promoter.

24. The cell according to any one of the preceding claims, wherein one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is codon-optimised for the cell.

25. The cell according to any one of the preceding claims, wherein one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is present in high copy number.

26. The cell according to any one of the preceding claims, wherein one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is integrated in the genome of the cell.

27. The cell according to any one of the preceding claims, wherein one or more of the genes encoding the tryptophan-2-halogenase, the tryptophan-5-halogenase, the tryptophan-6-halogenase, the tryptophan-7-halogenase, the flavin reductase, the tryptophan decarboxylase and/or the indole N-methyltransferase is expressed from a vector such as a plasmid.

28. The cell according to any one of the preceding claims, wherein the halogen is chlorine, fluorine, bromine or iodine, and wherein the cell is capable of producing, respectively, a chlorinated, a fluorinated, a brominated or a iodinated tryptophan, said cell expressing:
the tryptophan-5-halogenase SrPyrH (SEQ ID NO: 32), or a functional variant thereof having at least at least 80% homology, such as at least 85%, such as at least 90%, such as at least 95% homology thereto, and
the flavin reductase LaRebF (SEQ ID NO: 35), or a functional variant thereof having at least at least 80% homology, such as at least 85%, such as at least 90%, such as at least 95% homology thereto.

29. The cell according to any one of the preceding claims, wherein the halogen is chlorine, fluorine, bromine or iodine, and wherein the cell is capable of producing, respectively, a chlorinated, a fluorinated, a brominated or a iodinated tryptophan, said cell expressing:
the tryptophan-6-halogenase SttH (SEQ ID NO: 33), or a functional variant thereof having at least at least 80% homology, such as at least 85%, such as at least 90%, such as at least 95% homology thereto, and
the flavin reductase LaRebF (SEQ ID NO: 35), or a functional variant thereof having at least at least 80% homology, such as at least 85%, such as at least 90%, such as at least 95% homology thereto.

30. The cell according to any one of the preceding claims, wherein the halogen is chlorine, fluorine, bromine or iodine, and wherein the cell is capable of producing, respectively, a chlorinated, a fluorinated, a brominated or a iodinated tryptophan and/or, respectively, a chlorinated, a fluorinated, a brominated or a iodinated tryptamine, said cell expressing:
the tryptophan-5-halogenase SrPyrH (SEQ ID NO: 32), or a functional variant thereof having at least at least 80% homology, such as at least 85%, such as at least 90%, such as at least 95% homology thereto,
the flavin reductase LaRebF (SEQ ID NO: 35), or a functional variant thereof having at least at least 80% homology, such as at least 85%, such as at least 90%, such as at least 95% homology thereto, and
the tryptophan decarboxylase CrTDC (SEQ ID NO: 1) or a functional variant thereof having at least at least 80% homology, such as at least 85%, such as at least 90%, such as at least 95% homology thereto.

31. The cell according to any one of the preceding claims, wherein the halogen is chlorine, fluorine, bromine or iodine, and wherein the cell is capable of producing, respectively, a chlorinated, a fluorinated, a brominated or a iodinated tryptophan and/or, respectively, a chlorinated, a fluorinated, a brominated or a iodinated tryptamine, said cell expressing:
the tryptophan-6-halogenase SttH (SEQ ID NO: 33), or a functional variant thereof having at least at least 80% homology, such as at least 85%, such as at least 90%, such as at least 95% homology thereto,
the flavin reductase LaRebF (SEQ ID NO: 35), or a functional variant thereof having at least at least 80% homology, such as at least 85%, such as at least 90%, such as at least 95% homology thereto, and
the tryptophan decarboxylase CrTDC (SEQ ID NO: 1), or a functional variant thereof having at least at least 80% homology, such as at least 85%, such as at least 90%, such as at least 95% homology thereto.

32. A method of producing a halogenated tryptophan wherein the halogenated tryptophan is a tryptophan substituted with one, two or three halogen atoms, and optionally derivatives thereof, in a cell, preferably wherein the cell is a microorganism or a plant cell, said method comprising the steps of providing a cell and incubating said cell in the presence of a halogen, wherein the cell expresses at least one of:
a tryptophan-2-halogenase (EC 1.14.14), preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto,
a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto, or
a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto,
and optionally a flavin reductase, preferably a heterologous flavin reductase (EC: EC 1.5.1.30), such as LaRebF (SEQ ID NO: 35), or a functional variant thereof having at least 80% homology, such as at least 85%, such as at least 90%, such as at least 95% homology thereto.

33. The method according to claim 32, wherein the halogenated tryptophan is a 2-halogenated, a 5-halogenated, a 6-halogenated and/or a 7-halogenated tryptophan, a 2,5-dihalogenated, a 2,6-dihalogenated, a 2,7-dihalogenated, a 5,6-dihalogenated, a 5,7-dihalogenated and/or a 6,7-dihalogenated tryptophan, a 2,5,6-trihalogenated, a 2,5,7-trihalogenated, a 2,6,7-trihalogenated and/or a 5,6,7-trihalogenated tryptophan.

34. The method according to any one of claims 32 to 33, wherein the method is for producing a halogenated tryptamine and the cell further expresses a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting the halogenated tryptophan to a corresponding halogenated tryptamine.

35. The method according to claims 32 to 34, wherein the method is for producing a halogenated N-methyltryptamine, a halogenated N,N-dimethyltryptamine, and/or a halogenated N,N,N-trimethyltryptamine and the cell further expresses an indole N-methyltransferase (EC 2.1.1.49), preferably a heterologous indole N-methyltransferase, such as OcINMT (SEQ ID NO: 36) or a functional variant thereof having at least 80% homology, such as at least 85%, such as at least 90%, such as at least 95% homology thereto, wherein the indole N-methyltransferase is capable of converting the halogenated tryptamine to a corresponding halogenated N-methyltryptamine, a corresponding halogenated N,N-dimethyltryptamine, and/or a corresponding halogenated N,N,N-trimethyltryptamine.

36. The method according to any one of claims 32 to 35, wherein the cell is as defined in any one of claims 1 to 31.

37. The method according to any one of claims 32 to 36, wherein the 2-halogenated tryptophan, the 5-halogenated tryptophan, the 6-halogenated tryptophan, and/or the 7-halogenated tryptophan is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

38. The method according to any one of claims 32 to 37, wherein the 2,5-dihalogenated tryptophan, the 2,6-dihalogenated tryptophan, the 2,7-dihalogenated tryptophan, the 5,6-dihalogenated tryptophan, the 5,7-dihalogenated tryptophan and/or the 6,7-dihalogenated tryptophan is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

39. The method according to any one of claims 32 to 38, wherein the 2,5,6-trihalogenated tryptophan, the 2,5,7-trihalogenated tryptophan, the 2,6,7-trihalogenated tryptophan and/or the 5,6,7-trihalogenated is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

40. The method according to any one of claims 32 to 39, wherein the 2-halogenated tryptamine, the 5-halogenated tryptamine, the 6-halogenated tryptamine and/or the 7-halogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

41. The method according to any one of claims 32 to 40, wherein the 2,5-dihalogenated tryptamine, the 2,6-dihalogenated tryptamine, the 2,7-dihalogenated tryptamine, the 5,6-dihalogenated tryptamine, the 5,7-dihalogenated tryptamine, and/or the 6,7-dihalogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

42. The method according to any one of claims 32 to 41, wherein the 2,5,6-trihalogenated tryptamine, the 2,5,7-trihalogenated tryptamine, the 2,6,7-trihalogenated tryptamine and/or the 5,6,7-trihalogenated tryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

43. The method according to any one of claims 32 to 42, wherein the 2-halogenated N-methyltryptamine, the 2-halogenated N,N-dimethyltryptamine, the 2-halogenated N,N,N-trimethyltryptamine, the 5-halogenated N-methyltryptamine, the 5-halogenated N,N-dimethyltryptamine, the 5-halogenated N,N,N-trimethyltryptamine, the 6-halogenated N-methyltryptamine, the 6-halogenated N,N-dimethyltryptamine, the 6-halogenated N,N,N-trimethyltryptamine, the 7-halogenated N-methyltryptamine, the 7-halogenated N,N-dimethyltryptamine and/or the 7-halogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

44. The method according to any one of claims 32 to 43, wherein the 2,5-dihalogenated N-methyltryptamine, the 2,5-dihalogenated N,N-dimethyltryptamine, the 2,5-dihalogenated N,N,N-trimethyltryptamine, the 2,6-dihalogenated N-methyltryptamine, the 2,6-dihalogenated N,N-dimethyltryptamine, the 2,6-dihalogenated N,N,N-trimethyltryptamine, the 2,7-dihalogenated N-methyltryptamine, the 2,7-dihalogenated N,N-dimethyltryptamine, the 2,7-dihalogenated N,N,N-trimethyltryptamine, the 5,6-dihalogenated N-methyltryptamine, the 5,6-dihalogenated N,N-dimethyltryptamine, the 5,6-dihalogenated N,N,N-trimethyltryptamine, the 5,7-dihalogenated N-methyltryptamine, the 5,7-dihalogenated N,N-dimethyltryptamine, the 5,7-dihalogenated N,N,N-trimethyltryptamine, the 6,7-dihalogenated N-methyltryptamine, the 6,7-dihalogenated N,N-dimethyltryptamine and/or the 6,7-dihalogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

45. The method according to any one of claims 32 to 44, wherein the 2,5,6-trihalogenated N-methyltryptamine, the 2,5,6-trihalogenated N,N-dimethyltryptamine, the 2,5,6-trihalogenated N,N,N-trimethyltryptamine, the 2,5,7-trihalogenated N-methyltryptamine, the 2,5,7-trihalogenated N,N-dimethyltryptamine, the 2,5,7-trihalogenated N,N,N-trimethyltryptamine, the 2,6,7-trihalogenated N-methyltryptamine, the 2,6,7-trihalogenated N,N-dimethyltryptamine, the 2,6,7-trihalogenated N,N,N-trimethyltryptamine, the 5,6,7-trihalogenated N-methyltryptamine, the 5,6,7-trihalogenated N,N-dimethyltryptamine and/or the 5,6,7-trihalogenated N,N,N-trimethyltryptamine is produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

46. The method according to any one of claims to any one of claims 32 to 45, wherein the total titer of all produced halogenated compounds is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more, wherein the total titer is the sum of the titers of the 2-halogenated, 5-halogenated, 6-halogenated, 7-halogenated, 2,5-dihalogenated, 2,6-dihalogenated, 2,7-dihalogenated, 5,6-dihalogenated, 5,7-dihalogenated, 6,7-dihalogenated, 2,5,6-trihalogenated, 2,5,7-trihalogenated, 2,6,7-trihalogenated and 5,6,7-trihalogenated tryptophans, tryptamines, N-methyltryptamines, N,N-dimethyltryptamines and N,N,N-trimethyltryptamines.

47. The method according to any one of claims 32 to 46, further comprising a step of recovering the halogenated tryptophan, the dihalogenated tryptophan, the trihalogenated tryptophan, the halogenated tryptamine, the dihalogenated tryptamine, the trihalogenated tryptamine, the halogenated N-methyltryptamine, the halogenated N,N-dimethyltryptamine and/or the halogenated N,N,N-trimethyltryptamine, the dihalogenated N-methyltryptamine, the dihalogenated N,N-dimethyltryptamine and/or the dihalogenated N,N,N-trimethyltryptamine, the trihalogenated N-methyltryptamine, the trihalogenated N,N-dimethyltryptamine and/or the trihalogenated N,N,N-trimethyltryptamine.

48. A halogenated tryptophan, a dihalogenated tryptophan, a trihalogenated tryptophan, a halogenated tryptamine, a dihalogenated tryptamine, a trihalogenated tryptamine, a halogenated N-methyltryptamine, a halogenated N,N-dimethyltryptamine, a halogenated N,N,N-trimethyltryptamine, a dihalogenated N-methyltryptamine, a dihalogenated N,N-dimethyltryptamine, a dihalogenated N,N,N-trimethyltryptamine, a trihalogenated N-methyltryptamine, a trihalogenated N,N-dimethyltryptamine and/or a trihalogenated N,N,N-trimethyltryptamine obtainable by a method according to any one of claims 32 to 47.

49. A nucleic acid construct comprising at least one of:
a polynucleotide encoding a tryptophan-2-halogenase (EC 1.14.14),
preferably a heterologous tryptophan-2-halogenase such as CcCmdE (SEQ ID NO: 48) or a functional variant thereof having at least 80% homology, such as at least 85%, such as at least 90%, such as at least 95% homology thereto,
a polynucleotide encoding a tryptophan-5-halogenase (EC 1.14.19.58), preferably a heterologous tryptophan-5-halogenase such as SrPyrH (SEQ ID NO: 32) or a functional variant thereof having at least 80% homology, such as at least 85%, such as at least 90%, such as at least 95% homology thereto,
a polynucleotide encoding a tryptophan-6-halogenase (EC 1.14.19.59), preferably a heterologous tryptophan-6-halogenase such as SttH (SEQ ID NO: 33), SaThaI (SEQ ID NO: 51), or KtzR (SEQ ID NO: 54), or a functional variant thereof having at least 80% homology, such as at least 85%, such as at least 90%, such as at least 95% homology thereto, more preferably the tryptophan-6-halogenase is SttH (SEQ ID NO: 33) or a functional variant thereof having at least 80% homology thereto,
a polynucleotide encoding a tryptophan-7-halogenase (EC 1.14.19.9), preferably a heterologous tryptophan-7-halogenase such as LaRebH (SEQ ID NO: 34), PfPrnA (SEQ ID NO: 50), or KtzQ (SEQ ID NO: 53), or a functional variant thereof having at least 80% homology, such as at least 85%, such as at least 90%, such as at least 95% homology thereto, more preferably the tryptophan-7-halogenase is LaRebH (SEQ ID NO: 34) or a functional variant thereof having at least 80% homology thereto, or
a polynucleotide encoding a tryptophan halogenase, preferably a heterologous tryptophan halogenase such as DdChlA (SEQ ID NO: 52), or a functional variant thereof having at least 80% homology, such as at least 85%, such as at least 90%, such as at least 95% homology thereto,
and optionally a polynucleotide encoding a flavin reductase (EC 1.5.1.30), preferably a heterologous flavin reductase, such as LaRebF (SEQ ID NO: 35) or a functional variant thereof having at least 80% homology, such as at least 85%, such as at least 90%, such as at least 95% homology thereto.

50. The nucleic acid construct according to claim 49, further comprising a polynucleotide encoding a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto.

51. The nucleic acid construct according to any one of claims 49 to 50, further comprising a polynucleotide encoding an indole N-methyltransferase (EC 2.1.1.49), preferably a heterologous indole N-methyltransferase, such as OcINMT (SEQ ID NO: 36) or a functional variant thereof having at least 80% homology, such as at least 85%, such as at least 90%, such as at least 95% homology thereto.

52. The nucleic acid construct according to any one of claims 49 to 51, wherein the polynucleotide encoding the heterologous tryptophan-2-halogenase comprises or consists of SEQ ID NO: 49 or a variant thereof having at least 80% homology, such as at least 85%, such as at least 90%, such as at least 95% homology thereto.

53. The nucleic acid construct according to any one of claims 49 to 52, wherein the polynucleotide encoding the heterologous tryptophan-5-halogenase comprises or consists of SEQ ID NO: 37 or a variant thereof having at least 80% homology, such as at least 85%, such as at least 90%, such as at least 95% homology thereto.

54. The nucleic acid construct according to any one of claims 49 to 53, wherein the polynucleotide encoding the heterologous tryptophan-6-halogenase comprises or consists of SEQ ID NO: 38 or a variant thereof having at least 80% homology, such as at least 85%, such as at least 90%, such as at least 95% homology thereto.

55. The nucleic acid construct according to any one of claims 49 to 54, wherein the polynucleotide encoding the heterologous tryptophan-7-halogenase comprises or consists of SEQ ID NO: 39 or a variant thereof having at least 80% homology, such as at least 85%, such as at least 90%, such as at least 95% homology thereto.

56. The nucleic acid construct according to any one of claims 49 to 55, wherein the polynucleotide encoding the heterologous tryptophan halogenase comprises or consists of SEQ ID NO: 57 or a variant thereof having at least 80% homology, such as at least 85%, such as at least 90%, such as at least 95% homology thereto.

57. The nucleic acid construct according to any one of claims 49 to 56, wherein the polynucleotide encoding the heterologous flavin reductase comprises or consists of SEQ ID NO: 40 or a variant thereof having at least 80% homology, such as at least 85%, such as at least 90%, such as at least 95% homology thereto.

58. The nucleic acid construct according to any one of claims 49 to 57, wherein the polynucleotide encoding the heterologous tryptophan decarboxylase comprises or consists of SEQ ID NO: 6 or a variant thereof having at least 80% homology, such as at least 85%, such as at least 90%, such as at least 95% homology thereto.

59. The nucleic acid construct according to any one of 49 to 58, wherein the polynucleotide encoding the heterologous indole N-methyltransferase comprises or consists of SEQ ID NO: 41 or a variant thereof having at least 80% homology, such as at least 85%, such as at least 90%, such as at least 95% homology thereto.

60. The nucleic acid construct according to any one of claims 49 to 59, wherein one or more of the polynucleotides is/are codon-optimized for said cell.

61. The nucleic acid construct according to any one of claims 49 to 60, comprising or consisting of one or more vectors.

62. The cell according to any one of claims 1 to 31, wherein the cell comprises a nucleic acid construct according to any one of claims 49 to 61.

63. A kit of parts comprising:
the cell according to any one of claims 1 to 31 and instructions for use, preferably wherein the cell is a microorganism or a plant cell; and/or
the nucleic acid construct according to any one of claims 49 to 61 and instructions for use; and optionally the cell to be modified.

64. A cell capable of producing N-methyltryptamine, N,N-dimethyltryptamine and/or N,N,N-trimethyltryptamine, preferably wherein the cell is a microorganism or a plant cell, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine; and
an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, wherein the indole N-methyltransferase is capable of converting tryptamine to N-methyltryptamine, to N,N-dimethyltryptamine, and/or to N,N,N-trimethyltryptamine,
whereby the cell is capable of producing N-methyltryptamine, N,N-dimethyltryptamine, and/or N,N,N-trimethyltryptamine.

65. The cell according to claim 64, wherein the cell is as defined in any one of the preceding claims.

66. A method for producing N-methyltryptamine, N,N-dimethyltryptamine and/or N,N,N-trimethyltryptamine in a cell, preferably wherein the cell is a microorganism or a plant cell, said method comprising the steps of providing a cell and incubating said cell in a medium, wherein the cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine; and
an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, wherein the indole N-methyltransferase is capable of converting tryptamine to N-methyltryptamine, to N,N-dimethyltryptamine, and/or to N,N,N-trimethyltryptamine.

67. The method according to claim 66, wherein the cell is as defined in any one of the preceding claims.

68. The method according to any one of claims 66 to 67, further comprising a step of recovering the N-methyltryptamine, to N,N-dimethyltryptamine, and/or to N,N,N-trimethyltryptamine.

69. The method according to any one of claims 66 to 68, wherein N-methyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

70. The method according to any one of claims 66 to 69, wherein N,N-dimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

71. The method according to any one of claims 66 to 70, wherein N,N,N-trimethyltryptamine is produced with a titer of at least 20 mg/L, such as at least 30 mg/L, such as at least 40 mg/L, such as at least 50 mg/L, such as at least 60 mg/L, such as at least 70 mg/L, such as at least 80 mg/L, such as at least 90 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more, such as at least 6 g/L, such as at least 7 g/L, such as at least 8 g/L, such as at least 9 g/L, such as at least 10 g/L, such as at least 20 g/L, such as at least 30 g/L or more.

72. A nucleic acid construct for modifying a cell, wherein the cell is as defined in any one of the preceding claims, said construct comprising:
a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto, such as a first polynucleotide comprising or consisting of SEQ ID NO: 6 or a homologue thereof having at least 80% homology, such as at least 85%, such as at least 90%, such as at least 95% homology thereto
a seventh polynucleotide encoding an indole N-methyltransferase, preferably a heterologous indole N-methyltransferase (EC 2.1.1.49), such as OcINMT (SEQ ID NO: 36) or a functional variant thereof having at least 80% homology, such as at least 85%, such as at least 90%, such as at least 95% homology thereto, such as a seventh polynucleotide comprising or consisting of SEQ ID NO: 36 or a homologue thereof having at least 80% homology, such as at least 85%, such as at least 90%, such as at least 95% homology thereto.

73. A kit of parts comprising:
the cell according to any one of claims 64 to 65 and instructions for use, preferably wherein the cell is a microorganism or a plant cell, such as a cell as defined in any one of the preceding claims; and/or
the nucleic acid construct according to claim 72 and instructions for use; and
optionally the cell to be modified, preferably wherein the cell to be modified is a cell as defined in any one of the preceding claims.

74. A cell, preferably a yeast cell, capable of producing 4-hydroxytryptamine and optionally derivatives thereof, said cell expressing:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto,
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto,
wherein the tryptamine 4-monooxygenase and the cytochrome P450 reductase together catalyse the conversion of tryptamine to 4-hydroxytryptamine,
whereby the cell is capable of converting tryptophan to 4-hydroxytryptamine.

75. The cell according to claim 74, wherein the cell is a cell as defined in any one of the preceding claims.

76. The cell according to any one of claims 74 to 75, wherein the cell belongs to the genus of Aspergillus, e.g. A. niger, A. awamori, A. oryzae, A. nidulans, to the genus of Saccharomyces, such as S. cerevisiae, S. kluyveri, S. bayanus, S. exiguus, S. sevazzi, S. uvarum, S. boulardii, to the genus Kluyveromyces, such as K. lactis, K. marxianus var. marxianus, K. thermotolerans, to the genus Candida, such as C. utilis C. tropicalis, C. albicans, C. lipolytica, C. versatilis, to the genus Pichia, such as P. stipidis, P. pastoris, P. sorbitophila, other yeast genera such as Cryptococcus, such as C. aerius, Debaromyces, such as D. hansenii, Hansenula, Pichia, such as P. pastoris, Yarrowia, such as Y. lipolytica, Zygosaccharomyces, such as Z. bailii, Torulaspora, such as T. delbrueckii, Schizosaccharomyces, such as S. pombe, Brettanomyces, such as B. bruxellensis, Penicillium, Rhizopus, Fusarium, Fusidium, Gibberella, Mucor, Mortierella, or Trichoderma.

77. The cell according to any one of claims 74 to 76, wherein the cell is capable of producing 4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

78. The cell according to any one of claims 74 to 77, further expressing a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, wherein the 4-hydroxytryptamine kinase is capable of converting 4-hydroxytryptamine to norbaeocystin,
whereby the cell is capable of producing norbaeocystin, optionally wherein the cell is capable of producing norbaeocystin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

79. The cell according to any one of claims 74 to 78, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, whereby the yeast cell is capable of converting norbaeocystin to baeocystin, wherein optionally the baeocystin is converted spontaneously to norpsilocin, whereby the cell is capable of producing baeocystin and optionally norpsilocin, optionally wherein the cell is capable of producing baeocystin and optionally norpsilocin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

80. The cell according to any one of claims 74 to 79, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin and baeocystin to psilocybin, whereby the cell is capable of producing psilocybin, wherein optionally the psilocybin is converted spontaneously to psilocin, optionally wherein the cell is capable of producing psilocybin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

81. The cell according to any one of claims 74 to 80, further expressing a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, wherein the norbaeocystin N-methyl transferase/psilocybin synthase is capable of converting norbaeocystin to baeocystin, baeocystin to psilocybin, and psilocybin to aeruginascin,
whereby the cell is capable of converting norbaeocystin to aeruginascin,
wherein optionally the aeruginascin is converted spontaneously to dephosphorylated aeruginascin,
optionally wherein the cell is capable of producing aeruginascin and optionally dephosphorylated aeruginascin with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

82. The cell according to any one of claims 74 to 81, further expressing a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine, whereby the cell is capable of converting 4-hydroxytryptamine to N-acetyl-4-hydroxytryptamine, optionally wherein the cell is capable of producing N-acetyl-4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

83. The cell according to any one of claims 74 to 82, wherein the cell is capable of producing 4-hydroxytryptamine, norbaeocystin, baeocystin, psilocybin, psilocin, aeruginascin and/or N-acetyl-4-hydroxytryptamine with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

84. The cell according to any one of claims 74 to 83, further comprising one or more mutations resulting in increased availability of L-tryptophan, such as one or more mutations in one or more genes encoding transcriptional repressor(s) of genes of the aromatic amino acid precursor pathway such as RIC1, ARO1, ARO2, ARO3 or ARO4, or one or more mutations in the promoters of said genes, optionally wherein said mutation is a deletion.

85. The cell according to claim 84, wherein the one or more mutations is a mutation resulting in partial or total loss of activity of the one or more transcriptional repressor(s).

86. The cell according to any one of claims 84 to 85, wherein the one or more mutations is one or more of:
a mutation in ARO4 (SEQ ID NO: 12), such as a K229L mutation;
a mutation in ARO1 (SEQ ID NO: 13) and/or ARO2 (SEQ ID NO: 14);
a mutation in CDC19 (SEQ ID NO: 26);
a mutation in ARO8 (SEQ ID NO: 23) and/or ARO9 (SEQ ID NO: 24);
a mutation in GLT1 (SEQ ID NO: 25);
a mutation in TRP2 (SEQ ID NO: 16);
wherein the mutation is a mutation leading to a loss of function of the corresponding protein, such as a deletion.

87. The cell according to any one of claims 74 to 86, further overexpressing one or more of:
SER1 (SEQ ID NO: 20);
SER2 (SEQ ID NO: 21);
SER3 (SEQ ID NO: 22);
SER33 (SEQ ID NO: 23);
STB5 (SEQ ID NO: 26);
POS5 (SEQ ID NO: 27);
ZWF1 (SEQ ID NO: 29).

88. The cell according to any one of claims 74 to 87, further expressing a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto, and/or comprising one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).

89. A method of producing 4-hydroxytryptamine and optionally derivatives thereof in a cell such as a yeast cell, said method comprising the steps of providing a cell and incubating said cell in a medium, wherein the cell expresses:
a tryptophan decarboxylase (EC 4.1.1.105), preferably a heterologous tryptophan decarboxylase such as CrTDC (SEQ ID NO: 1) or a functional variant thereof having at least 80% homology, such as at least 85%, such as at least 90%, such as at least 95% homology thereto, wherein the tryptophan decarboxylase is capable of converting tryptophan to tryptamine;
a tryptamine 4-monooxygenase (EC 1.14.99.59), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, and
a cytochrome P450 reductase (EC 1.6.2.4), preferably a heterologous cytochrome P450 reductase such as PcCpr (SEQ ID NO: 3) or a functional variant thereof having at least 80% homology, such as at least 85%, such as at least 90%, such as at least 95% homology thereto.

90. The method according to claim 89, wherein the method is for producing norbaeocystin and the cell further expresses a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto.

91. The method according to any one of claims 89 to 90, wherein the method is for producing baeocystin and optionally norpsilocin and the cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.

92. The method according to any one of claims 89 to 91, wherein the method is for producing N-acetyl-4-hydroxytryptamine and the cell further expresses a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, wherein the serotonin N-acetyltransferase is capable of converting 4-hydroxytryptamine to N-acetyl-4-hydroxytryptamine.

93. The method according to any one of claims 89 to 92, wherein the method is for producing psilocybin and optionally psilocin and/or aeruginascin and optionally dephosphorylated aeruginascin and the cell further expresses a norbaeocystin N-methyl transferase/psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto.

94. The method according to any one of claims 89 to 93, wherein the medium comprises tryptophan and/or wherein the cell is capable of synthesising tryptophan.

95. The method according to any one of claims 89 to 94, wherein the cell is as defined in any one of the preceding claims.

96. The method according to any one of claims 89 to 95, wherein the medium is supplemented with at least 1 g/L glutamine, such as at least 2 g/L, such as at least 3 g/L glutamine, such as at least 4 g/L glutamine, such as at least 5 g/L glutamine, such as at least 6 g/L glutamine, such as at least 7 g/L glutamine, such as at least 8 g/L glutamine, such as at least 9 g/L glutamine, such as at least 10 g/L glutamine, or more.

97. The method according to any one of claims 89 to 96, wherein 4-hydroxytryptamine, norbaeocystin, baeocystin and optionally norpsilocin, N-acetyl-4-hydroxytryptamine, psilocybin and optionally psilocin, aeruginascin and optionally dephosphorylated aeruginascinis produced with a titer of at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 20 g/L, such as at least 30 g/L or more.

98. The method according to any one of claims 89 to 97, further comprising a step of recovering the 4-hydroxytryptamine, the norbaeocystin, the norpsilocin, the N-acetyl-4-hydroxytryptamine, the psilocybin and optionally the psilocin, and/or the aeruginascin and optionally the dephosphorylated aeruginascin.

99. The method according to any one of claims 89 to 98, wherein the yeast cell further expresses a cytochrome b5 such as PcCyb5 (SEQ ID NO: 43), or a functional variant thereof having at least 85%, such as at least 90%, such as at least 95%, such as at least 96%, such as at least 97%, such as at least 98%, or such as at least 99% homology thereto.

100. The method according to any one of claims 89 to 99, wherein the yeast cell further comprises one or more mutations resulting in increased availability of S-adenosylmethionine (SAM), such as a deletion or a mutation of ERG4 (SEQ ID NO: 45) and/or a deletion or a mutation of SPE2 (SEQ ID NO: 47) resulting in partial or total loss of Erg4 (SEQ ID NO: 44) and/or Spe2 (SEQ ID NO: 46).

101. 4-hydroxytryptamine, norbaeocystin, baeocystin, norpsilocin, psilocybin, psilocin, aeruginascin, dephosphorylated aeruginascin or N-acetyl-4-hydroxytryptamine obtainable by a method according to any one of claims 89 to 100.

102. A nucleic acid construct for modifying a cell, wherein the cell preferably is a yeast cell, said construct comprising:
a first polynucleotide encoding a tryptophan decarboxylase (EC 4.1.1.105) (SEQ ID NO: 6), preferably a heterologous tryptophan decarboxylase such as CrTDC (SEQ ID NO: 1) or a functional variant thereof having at least 80% homology, such as at least 85%, such as at least 90%, such as at least 95% homology thereto, such as a first polynucleotide comprising or consisting of SEQ ID NO: 6 or a homologue thereof having at least 80% homology, such as at least 85%, such as at least 90%, such as at least 95% homology thereto
a second polynucleotide encoding a tryptamine 4-monooxygenase (EC 1.14.99.59) (SEQ ID NO: 7), preferably a heterologous tryptamine 4-monooxygenase such as PcPsiH (SEQ ID NO: 2) or a functional variant thereof having at least 80% homology, such as at least 85%, such as at least 90%, such as at least 95% homology thereto, such as a second polynucleotide comprising or consisting of SEQ ID NO: 7 or a homologue thereof having at least 80% homology, such as at least 85%, such as at least 90%, such as at least 95% homology thereto; and
a third polynucleotide encoding a cytochrome P450 reductase (EC 1.6.2.4) (SEQ ID NO:8), preferably a heterologous cytochrome P450 reductase such as PcCpr (SEQ ID NO: 3) or a functional variant thereof having at least 80% homology, such as at least 85%, such as at least 90%, such as at least 95% homology thereto, such as a third polynucleotide comprising or consisting of SEQ ID NO: 8 or a homologue thereof having at least 80% homology, such as at least 85%, such as at least 90%, such as at least 95% homology thereto; and optionally,
a fourth polynucleotide encoding a 4-hydroxytryptamine kinase (EC 2.7.1.222), preferably a heterologous 4-hydroxytryptamine kinase such as PcPsiK (SEQ ID NO: 4) or a functional variant thereof having at least 80% homology, such as at least 85%, such as at least 90%, such as at least 95% homology thereto, such as a fourth polynucleotide comprising or consisting of SEQ ID NO: 9 or a homologue thereof having at least 80% homology, such as at least 85%, such as at least 90%, such as at least 95% homology thereto; and
a fifth polynucleotide encoding a psilocybin synthase (EC 2.1.1.345), preferably a heterologous psilocybin synthase such as PcPsiM (SEQ ID NO: 5) or a functional variant thereof having at least 80% homology, such as at least 85%, such as at least 90%, such as at least 95% homology thereto, such as a fifth polynucleotide comprising or consisting of SEQ ID NO: 10 or a homologue thereof having at least 80% homology, such as at least 85%, such as at least 90%, such as at least 95% homology thereto; and/or
a sixth polynucleotide encoding a serotonin N-acetyltransferase (EC 2.3.1.87), preferably a heterologous serotonin N-acetyltransferase such as BtAANAT (SEQ ID NO: 11) or a functional variant thereof having at least 80% homology, such as at least 85%, such as at least 90%, such as at least 95% homology thereto, such as a sixth polynucleotide comprising or consisting of SEQ ID NO: 30 or a homologue thereof having at least 80% homology, such as at least 85%, such as at least 90%, such as at least 95% homology thereto.

103. The cell according to any one of claims 1 to 31, 62, 64 to 65, or 74 to 88, wherein the cell is a yeast cell, for use as a probiotic.

104. A composition or a pharmaceutical composition comprising the cell according to any one of claims 1 to 31, 62, 64 to 65, or 74 to 88, wherein the cell is a yeast cell.

105. Use of a cell according to any one of claims 1 to 31, 62, 64 to 65, or 74 to 88, wherein the cell is a yeast cell, or use of the composition or pharmaceutical composition according to claim 104, as a probiotic.

106. The composition or pharmaceutical composition according to claim 104 for use as a probiotic.

107. The cell according to any one of claims 1 to 31, 62, 64 to 65, or 74 to 88, wherein the cell is a yeast cell, or the composition or pharmaceutical composition according to claim 104 for use as a medicament, optionally wherein the cell further comprises means for biocontainment.

108. The cell, the composition or pharmaceutical composition for the use according to claim 107, wherein the composition or pharmaceutical composition is for oral administration.

109. The cell according to any one of claims 1 to 31, 62, 64 to 65, or 74 to 88, wherein the cell is a yeast cell, or the composition or pharmaceutical composition according to claim 104 for use in a method of treatment or prophylaxis of a disease or disorder selected from the group consisting of depression, such as major depressive disorder or treatment-resistant depression, anxiety disorders, obsessive-compulsive disorder, post-traumatic stress disorder, substance addiction or dependence such as alcohol or tobacco addiction or dependence, migraine and headache, preferably chronic migraines and chronic headaches.

110. The cell, the composition or the pharmaceutical composition for the use according to any one of claims 107 to 109, wherein the cell, the composition or the pharmaceutical composition is administered to a subject in need thereof.

111. The cell, the composition or the pharmaceutical composition for the use according to any one of claims 107 to 110, wherein the cell, the composition or the pharmaceutical composition is administered one to five times a day, such as once daily, twice daily, thrice daily, four times daily or five times daily, or every second day, every third day, once a week, every second week, or once a month.

112. The cell, the composition or the pharmaceutical composition for the use according to any one of claims 107 to 111, wherein the cell, the composition or the pharmaceutical composition is administered to a subject in need thereof in an amount such that one or more compounds are delivered to the subject, wherein said one or more compounds are selected from the group consisting of: a halogenated tryptophan, a halogenated tryptamine, a halogenated, di-halogenated or tri-halogenated N-methyltryptamine, a halogenated, di-halogenated or tri-halogenated N,N-dimethyltryptamine, a halogenated, di-halogenated or tri-halogenated N,N,N-trimethyltryptamine, N-methyltryptamine, N,N-dimethyltryptamine and/or N,N,N-trimethyltryptamine, norbaeocystin, baeocystin, norpsilocin, psilocybin, psilocin, aeruginascin, dephosphorylated aeruginascin, N-acetyl-4-hydroxytryptamine, and derivatives thereof, preferably as defined in any one of the preceding claims.

113. The cell, the composition or the pharmaceutical composition for the use according to claim 112, wherein the cell, the composition or the pharmaceutical composition is administered to the subject in an amount such that the one or more compounds are delivered to the subject's intestine in an amount in the range of 1 ng to 1 mg, such as between 1 ng and 750 μg, such as between 5 ng and 500 μg, such as between 10 ng and 250 μg, such as between 25 ng and 100 μg, such as between 50 ng and 75 μg, such as between 75 ng and 50 μg, such as between 50 ng and 25 μg, such as between 75 ng and 10 μg, such as between 100 ng and 7.5 μg, such as between 250 ng and 5 μg, such as between 500 ng and 2.5 μg, such as between 750 ng and 1 μg.

114. A method for increasing empathy and/or creativity, comprising administering to a subject the cell according to any one of claims 1 to 31, 62, 64 to 65, or 74 to 88, wherein the cell is a yeast cell, or the composition or pharmaceutical composition according to claim 104, optionally wherein the cell further comprises means for biocontainment.

115. The method according to claim 114, wherein the cell, the composition or the pharmaceutical composition is for oral administration.

116. The method according to any one of claims 114 to 115, wherein the cell, the composition or the pharmaceutical composition is administered one to five times a day, such as once daily, twice daily, thrice daily, four times daily or five times daily, or every second day, every third day, once a week, every second week, or once a month.

117. The method according to any one of claims 114 to 116, wherein the cell, the composition or the pharmaceutical composition is administered to a subject in need thereof in an amount such that one or more compounds are delivered to the subject, wherein said one or more compounds are selected from the group consisting of: a halogenated tryptophan, a halogenated tryptamine, a halogenated, di-halogenated or tri-halogenated N-methyltryptamine, a halogenated, di-halogenated or tri-halogenated N,N-dimethyltryptamine, a halogenated, di-halogenated or tri-halogenated N,N,N-trimethyltryptamine, N-methyltryptamine, N,N-dimethyltryptamine and/or N,N,N-trimethyltryptamine, norbaeocystin, baeocystin, norpsilocin, psilocybin, psilocin, aeruginascin, dephosphorylated aeruginascin, N-acetyl-4-hydroxytryptamine, and derivatives thereof, preferably as defined in any one of the preceding claims.

118. The method according to claim 117, wherein the cell, the composition or the pharmaceutical composition is administered to the subject in an amount such that the one or more compounds are delivered to the subject's intestine in an amount in the range of 1 ng to 1 mg, such as between 1 ng and 750 μg, such as between 5 ng and 500 μg, such as between 10 ng and 250 μg, such as between 25 ng and 100 μg, such as between 50 ng and 75 μg, such as between 75 ng and 50 μg, such as between 50 ng and 25 μg, such as between 75 ng and 10 μg, such as between 100 ng and 7.5 μg, such as between 250 ng and 5 μg, such as between 500 ng and 2.5 μg, such as between 750 ng and 1 μg.
